SEQUENCE LISTING (224 sequences)

SEQ ID NO: 1 - DNA, 60 bp, Artificial Sequence
  Synthetic
  N_region (14)..(20): n is A, C, G, or T
  N_region (14)..(20): n is a, c, g, or t
  misc_feature (24)..(30): n is a, c, g, or t
gtgccagcaagatccaatctagannnnnnntcactggactttggttatcttcgcaataag

SEQ ID NO: 2 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnnagacagcttcggcgcatccttttg

SEQ ID NO: 3 - DNA, 41 bp, Artificial Sequence
  Synthetic
aacggctgccacgtggaattgctccaacccttagggaaccc

SEQ ID NO: 4 - DNA, 50 bp, Artificial Sequence
  Synthetic
cccctgtatgaggttgatgacttacgagacgtccaacccttagggaaccc

SEQ ID NO: 5 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnncctccgagagacccgccctcgcccg

SEQ ID NO: 6 - DNA, 44 bp, Artificial Sequence
  Synthetic
agccagccctcctccctggccatgctccaacccttagggaaccc

SEQ ID NO: 7 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctgccttgcataaggccataatggttaaag

SEQ ID NO: 8 - DNA, 49 bp, Artificial Sequence
  Synthetic
gtgtggatgaagcaaccatcattgacattctccaacccttagggaaccc

SEQ ID NO: 9 - DNA, 50 bp, Artificial Sequence
  Synthetic
  N_region (14)..(20): n is any of a, c, g, or t
  misc_feature (24)..(30): n is a, c, g, or t
gtgccagcaagatccaatctagannnnnnngcacgaggcctgcatgagcg

SEQ ID NO: 10 - DNA, 49 bp, Artificial Sequence
  Synthetic
ggagttccgaatgtgtgaggcttcttattgtccaacccttagggaaccc

SEQ ID NO: 11 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctttgtcacagcccaagatagttaagtggg

SEQ ID NO: 12 - DNA, 47 bp, Artificial Sequence
  Synthetic
atcgagacatgtaagcagcatcatggagtccaacccttagggaaccc

SEQ ID NO: 13 - DNA, 59 bp, Artificial Sequence
  Synthetic
  N_region (14)..(20): n is any of a, c, g, or t
  misc_feature (24)..(30): n is a, c, g, or t
gtgccagcaagatccaatctagannnnnnngaaaaagtggcctggaaatgattcagcag

SEQ ID NO: 14 - DNA, 49 bp, Artificial Sequence
  Synthetic
gagaaattacgacaactacgagactgcatttccaacccttagggaaccc

SEQ ID NO: 15 - DNA, 56 bp, Artificial Sequence
  Synthetic
  misc_feature (24)..(30): n is a, c, g, or t
gtgccagcaagatccaatctagannnnnnncctggatccaggataacggaggctgg

SEQ ID NO: 16 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnagaggatcatgctgtacttaaaaaatacaa

SEQ ID NO: 17 - DNA, 44 bp, Artificial Sequence
  Synthetic
gatgcctttgtggaactgtacggcctccaacccttagggaaccc

SEQ ID NO: 18 - DNA, 49 bp, Artificial Sequence
  Synthetic
catcacagaggaagtagactgatattaacatccaacccttagggaaccc

SEQ ID NO: 19 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnncataaaacggtcctcatggcctgcag

SEQ ID NO: 20 - DNA, 60 bp, Artificial Sequence
  Synthetic
  misc_feature (24)..(30): n is a, c, g, or t
gtgccagcaagatccaatctagannnnnnnaagaagtttctaggaaaggccggacaccag

SEQ ID NO: 21 - DNA, 53 bp, Artificial Sequence
  Synthetic
tggcctgttctatagcatctttacagaccagttgtccaacccttagggaaccc

SEQ ID NO: 22 - DNA, 49 bp, Artificial Sequence
  Synthetic
gttttgagcaaaattttggactgtgaagcatccaacccttagggaaccc

SEQ ID NO: 23 - DNA, 60 bp, Artificial Sequence
  Synthetic
  misc_feature (24)..(30): n is a, c, g, or t
gtgccagcaagatccaatctagannnnnnnaaaaataggtgattttggtctagctacaga

SEQ ID NO: 24 - DNA, 42 bp, Artificial Sequence
  Synthetic
gaaatctcgatggagtgggtccctccaacccttagggaaccc

SEQ ID NO: 25 - DNA, 57 bp, Artificial Sequence
  Synthetic
  misc_feature (24)..(30): n is a, c, g, or t
gtgccagcaagatccaatctagannnnnnnccactcggagattctccaccattgtgg

SEQ ID NO: 26 - DNA, 40 bp, Artificial Sequence
  Synthetic
tggaggaaggccacgagggcctccaacccttagggaaccc

SEQ ID NO: 27 - DNA, 59 bp, Artificial Sequence
  Synthetic
  N_region (14)..(20): n is any of a, c, g, or t
  misc_feature (24)..(30): n is a, c, g, or t
gtgccagcaagatccaatctagannnnnnngacgacgcattcaggagaagaaggatgag

SEQ ID NO: 28 - DNA, 49 bp, Artificial Sequence
  Synthetic
gacatagctcgccttttgcaagaaaaggagtccaacccttagggaaccc

SEQ ID NO: 29 - DNA, 55 bp, Artificial Sequence
  Synthetic
  misc_feature (24)..(30): n is a, c, g, or t
gtgccagcaagatccaatctagannnnnnnaccttcgttgccctctgtgccacag

SEQ ID NO: 30 - DNA, 46 bp, Artificial Sequence
  Synthetic
atgtgaagttcatttccaatccgcccttccaacccttagggaaccc

SEQ ID NO: 31 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnggtggccacctggatgctggag

SEQ ID NO: 32 - DNA, 49 bp, Artificial Sequence
  Synthetic
gtctgtgaggaacagaagtgcgaagaagagtccaacccttagggaaccc

SEQ ID NO: 33 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnncctcagagccgctttcagaaaagcaag

SEQ ID NO: 34 - DNA, 43 bp, Artificial Sequence
  Synthetic
ctgcttctggttgggcccagaccttccaacccttagggaaccc

SEQ ID NO: 35 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnngtggtggctctccttgtcattttccag

SEQ ID NO: 36 - DNA, 47 bp, Artificial Sequence
  Synthetic
gtatgcctgtgtcaagatgaggtcacggtccaacccttagggaaccc

SEQ ID NO: 37 - DNA, 59 bp, Artificial Sequence
  Synthetic
  N_region (14)..(20): n is any of a, c, g, or t
  misc_feature (24)..(30): n is a, c, g, or t
gtgccagcaagatccaatctagannnnnnnagagcaagtggcctctgtaatctgctcag

SEQ ID NO: 38 - DNA, 46 bp, Artificial Sequence
  Synthetic
gaaaccagtcccaaacactgtcctcgttccaacccttagggaaccc

SEQ ID NO: 39 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnctggagatcactgctcggccag

SEQ ID NO: 40 - DNA, 46 bp, Artificial Sequence
  Synthetic
tactatggcactggctgctgaggactgtccaacccttagggaaccc

SEQ ID NO: 41 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnggatggaacgaatacacctcaatgtctctg

SEQ ID NO: 42 - DNA, 49 bp, Artificial Sequence
  Synthetic
aaaggccttttccacctcatatccagctcctccaacccttagggaaccc

SEQ ID NO: 43 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctcagcccacccacttaccttatgtcagtg

SEQ ID NO: 44 - DNA, 43 bp, Artificial Sequence
  Synthetic
agatgctggaggccaggacagctgtccaacccttagggaaccc

SEQ ID NO: 45 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnaaaaccatacagctgaattggtcatcccag

SEQ ID NO: 46 - DNA, 49 bp, Artificial Sequence
  Synthetic
aactacctctggcacatcctccaaatgaaatccaacccttagggaaccc

SEQ ID NO: 47 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnntcaacttattcccttcaattcaagtaacag

SEQ ID NO: 48 - DNA, 47 bp, Artificial Sequence
  Synthetic
gaaacaagattttggtgaagcagtcgcctccaacccttagggaaccc

SEQ ID NO: 49 - DNA, 50 bp, Artificial Sequence
  Synthetic
  N_region (14)..(20): n is any of a, c, g, or t
  misc_feature (24)..(30): n is a, c, g, or t
gtgccagcaagatccaatctagannnnnnntgctggcggcaggcaaaggg

SEQ ID NO: 50 - DNA, 44 bp, Artificial Sequence
  Synthetic
gacaaaacaaggagaggccaccacctccaacccttagggaaccc

SEQ ID NO: 51 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnngaggaggtgcaattgctagtgttcggat

SEQ ID NO: 52 - DNA, 44 bp, Artificial Sequence
  Synthetic
tgactgccaactctgacacccaccttccaacccttagggaaccc

SEQ ID NO: 53 - DNA, 53 bp, Artificial Sequence
  Synthetic
  N_region (14)..(20): n is any of a, c, g, or t
  misc_feature (24)..(30): n is a, c, g, or t
gtgccagcaagatccaatctagannnnnnncggctttggggtcaagcagattg

SEQ ID NO: 54 - DNA, 48 bp, Artificial Sequence
  Synthetic
ctacaggggtttctgataccatctgcgagtccaacccttagggaaccc

SEQ ID NO: 55 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnagaaagaaaacagctttgaaatgcaaaaag

SEQ ID NO: 56 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnattaaaagccagtttgaaggctttgtgaag

SEQ ID NO: 57 - DNA, 49 bp, Artificial Sequence
  Synthetic
tgttacagtgggctgaaaaaggatactacatccaacccttagggaaccc

SEQ ID NO: 58 - DNA, 49 bp, Artificial Sequence
  Synthetic
gatataatgttaaacaaagaggagacgaagtccaacccttagggaaccc

SEQ ID NO: 59 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnnccaccacaactccagagcccacag

SEQ ID NO: 60 - DNA, 40 bp, Artificial Sequence
  Synthetic
ctcctcccaggctgcagctggtccaacccttagggaaccc

SEQ ID NO: 61 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnnatgtacacaacccagggtggaggagag

SEQ ID NO: 62 - DNA, 45 bp, Artificial Sequence
  Synthetic
gcctggggcatctctgtactgaaccctccaacccttagggaaccc

SEQ ID NO: 63 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnngtagctgagctgcagctgaatcacacag

SEQ ID NO: 64 - DNA, 47 bp, Artificial Sequence
  Synthetic
gacctcagcaggaccccaggctatactgtccaacccttagggaaccc

SEQ ID NO: 65 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnngaaatttatcataaccggtttgatgctgtg

SEQ ID NO: 66 - DNA, 42 bp, Artificial Sequence
  Synthetic
caatctgcacatcgtgctgccactccaacccttagggaaccc

SEQ ID NO: 67 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnnagtattctggaaaacggtttcccgcagg

SEQ ID NO: 68 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnntctttgtgatggccttcctgctctctg

SEQ ID NO: 69 - DNA, 45 bp, Artificial Sequence
  Synthetic
tttgcagaagctgcctgtgatgtggttccaacccttagggaaccc

SEQ ID NO: 70 - DNA, 49 bp, Artificial Sequence
  Synthetic
gtgctgctcctctgaagattcaagcttatttccaacccttagggaaccc

SEQ ID NO: 71 - DNA, 51 bp, Artificial Sequence
  Synthetic
  N_region (14)..(20): n is any of a, c, g, or t
  misc_feature (24)..(30): n is a, c, g, or t
gtgccagcaagatccaatctagannnnnnntcgtgccggtcttcctgccag

SEQ ID NO: 72 - DNA, 38 bp, Artificial Sequence
  Synthetic
cgaagcccaccacgacgcctccaacccttagggaaccc

SEQ ID NO: 73 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnngccttctacagaacacagctggtttcctgg

SEQ ID NO: 74 - DNA, 49 bp, Artificial Sequence
  Synthetic
gtatgcctggactgttgcccagtgtaagattccaacccttagggaaccc

SEQ ID NO: 75 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnngaggaacctcctctggaaatgaacactggg

SEQ ID NO: 76 - DNA, 49 bp, Artificial Sequence
  Synthetic
gttgattcctcgtgccagaccattattccttccaacccttagggaaccc

SEQ ID NO: 77 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctcctcacagctgtttctttgagcaaaatg

SEQ ID NO: 78 - DNA, 49 bp, Artificial Sequence
  Synthetic
ctaaagaaaagaagccctcttacaacagggtccaacccttagggaaccc

SEQ ID NO: 79 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnnggtcagcagcctctctccagtccaag

SEQ ID NO: 80 - DNA, 54 bp, Artificial Sequence
  Synthetic
gtgttctggaggtctattacacaagcttgaggtgttccaacccttagggaaccc

SEQ ID NO: 81 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnnggacctcgagaacctggaggacctg

SEQ ID NO: 82 - DNA, 53 bp, Artificial Sequence
  Synthetic
ttctgggaactggacagattggacaactataacgtccaacccttagggaaccc

SEQ ID NO: 83 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnggaatgattgctgggggcacag

SEQ ID NO: 84 - DNA, 44 bp, Artificial Sequence
  Synthetic
gcatcacacccatgttgcagctcattccaacccttagggaaccc

SEQ ID NO: 85 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnncacgatagtgccaggcctatgttggag

SEQ ID NO: 86 - DNA, 48 bp, Artificial Sequence
  Synthetic
ggagttaaatacctgtgcatcccagcagctccaacccttagggaaccc

SEQ ID NO: 87 - DNA, 50 bp, Artificial Sequence
  Synthetic
  N_region (14)..(20): n is any of a, c, g, or t
  misc_feature (24)..(30): n is a, c, g, or t
gtgccagcaagatccaatctagannnnnnngtagccacccgtcccgggta

SEQ ID NO: 88 - DNA, 39 bp, Artificial Sequence
  Synthetic
caagtcccgggtggtgaggatccaacccttagggaaccc

SEQ ID NO: 89 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnnaattctgccataagccctgtcctccag

SEQ ID NO: 90 - DNA, 49 bp, Artificial Sequence
  Synthetic
gtgaaaggaaagctagggactgcacagtcatccaacccttagggaaccc

SEQ ID NO: 91 - DNA, 53 bp, Artificial Sequence
  Synthetic
  N_region (14)..(20): n is any of a, c, g, or t
  misc_feature (24)..(30): n is a, c, g, or t
gtgccagcaagatccaatctagannnnnnngatggagttgcaggtgtccagcg

SEQ ID NO: 92 - DNA, 42 bp, Artificial Sequence
  Synthetic
gctttgtgtgcaacacgtgcccttccaacccttagggaaccc

SEQ ID NO: 93 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnnaaccacacataccagctggatgtcgtgg

SEQ ID NO: 94 - DNA, 39 bp, Artificial Sequence
  Synthetic
agcggtcccctcaccggccctccaacccttagggaaccc

SEQ ID NO: 95 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnncccttccccttcaacctcttgctcaag

SEQ ID NO: 96 - DNA, 46 bp, Artificial Sequence
  Synthetic
gcatgattccaacagaactgcagcagctccaacccttagggaaccc

SEQ ID NO: 97 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnnggacaggccacatttcatgcaccag

SEQ ID NO: 98 - DNA, 42 bp, Artificial Sequence
  Synthetic
ctctcaacggtggatgcccacgctccaacccttagggaaccc

SEQ ID NO: 99 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnncctcattaagcccaagcgaaggctg

SEQ ID NO: 100 - DNA, 42 bp, Artificial Sequence
  Synthetic
tctgcagccaggagagcagggactccaacccttagggaaccc

SEQ ID NO: 101 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnaacttctccaacgacatcatgctactgcag

SEQ ID NO: 102 - DNA, 45 bp, Artificial Sequence
  Synthetic
ctggagagaaaggccaagcggaccagtccaacccttagggaaccc

SEQ ID NO: 103 - DNA, 74 bp, Artificial Sequence
  Synthetic
  N_region (14)..(20): n is any of a, c, g, or t
  misc_feature (24)..(30): n is a, c, g, or t
gtgccagcaagatccaatctagannnnnnncctggcggcctcagggctgtttcgatgcttgcctgtgtcatgcc

SEQ ID NO: 104 - DNA, 61 bp, Artificial Sequence
  Synthetic
cggaggacctgctggtggaggaattggtggacgggctattattccaacccttagggaaccc

SEQ ID NO: 105 - DNA, 67 bp, Artificial Sequence
  Synthetic
  N_region (14)..(20): n is any of a, c, g, or t
  misc_feature (24)..(30): n is a, c, g, or t
gtgccagcaagatccaatctagannnnnnnaaagtaactcttacaggaggatatttgcatatttatg

SEQ ID NO: 106 - DNA, 50 bp, Artificial Sequence
  Synthetic
aatcacaactttgttgccagctgaagttctgtccaacccttagggaaccc

SEQ ID NO: 107 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnccaagcccatcaccattggcaa

SEQ ID NO: 108 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnccaagcccatcaccattggcac

SEQ ID NO: 109 - DNA, 39 bp, Artificial Sequence
  Synthetic
gcacgcccatggcgaccagttccaacccttagggaaccc

SEQ ID NO: 110 - DNA, 65 bp, Artificial Sequence
  Synthetic
  N_region (14)..(20): n is any of a, c, g, or t
  misc_feature (24)..(30): n is a, c, g, or t
gtgccagcaagatccaatctagannnnnnnaacgagatgacttcgaaaagctgactaattattcg

SEQ ID NO: 111 - DNA, 49 bp, Artificial Sequence
  Synthetic
gtaactgacttgaatgtccaacgcaaagcatccaacccttagggaaccc

SEQ ID NO: 112 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnngcaccctggtcaccgtctcctcag

SEQ ID NO: 113 - DNA, 51 bp, Artificial Sequence
  Synthetic
  misc_feature (24)..(30): n is a, c, g, or t
gtgccagcaagatccaatctagannnnnnnagtgaccaggcgcccgacatg

SEQ ID NO: 114 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnnctctcagccaggaccaaggacagcag

SEQ ID NO: 115 - DNA, 51 bp, Artificial Sequence
  Synthetic
  N_region (14)..(20): n is any of a, c, g, or t
  misc_feature (24)..(30): n is a, c, g, or t
gtgccagcaagatccaatctagannnnnnngccctccagcagcctgaccag

SEQ ID NO: 116 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnncaaatggacgacccggtgcttcag

SEQ ID NO: 117 - DNA, 39 bp, Artificial Sequence
  Synthetic
ggagtgcatccgccccaacctccaacccttagggaaccc

SEQ ID NO: 118 - DNA, 41 bp, Artificial Sequence
  Synthetic
cttccaccaagggcccatcggttccaacccttagggaaccc

SEQ ID NO: 119 - DNA, 39 bp, Artificial Sequence
  Synthetic
catccccgaccagccccaagtccaacccttagggaaccc

SEQ ID NO: 120 - DNA, 44 bp, Artificial Sequence
  Synthetic
cctccacacagagcccatccgtctttccaacccttagggaaccc

SEQ ID NO: 121 - DNA, 53 bp, Artificial Sequence
  Synthetic
  N_region (14)..(20): n is any of a, c, g, or t
  misc_feature (24)..(30): n is a, c, g, or t
gtgccagcaagatccaatctagannnnnnngttgatggcgctgagagaacccg

SEQ ID NO: 122 - DNA, 39 bp, Artificial Sequence
  Synthetic
ctgcgcaggcacccgtcaagtccaacccttagggaaccc

SEQ ID NO: 123 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnngcgtcctccatgtgtggccccg

SEQ ID NO: 124 - DNA, 45 bp, Artificial Sequence
  Synthetic
atcaagacacagccatccgggtcttctccaacccttagggaaccc

SEQ ID NO: 125 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnnaggtgctcagcgatgctggacacaag

SEQ ID NO: 126 - DNA, 49 bp, Artificial Sequence
  Synthetic
gtcaccatcctggaggcagataacaggatctccaacccttagggaaccc

SEQ ID NO: 127 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnnctgccgaagccttggcgttctcag

SEQ ID NO: 128 - DNA, 44 bp, Artificial Sequence
  Synthetic
actgccggctgcacatctgcctgtatccaacccttagggaaccc

SEQ ID NO: 129 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnnggatccagctggcaggacacgcag

SEQ ID NO: 130 - DNA, 46 bp, Artificial Sequence
  Synthetic
ggagtttcaaggcagctgccaatggcatccaacccttagggaaccc

SEQ ID NO: 131 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnncaagaccagatggatgcccagatgag

SEQ ID NO: 132 - DNA, 49 bp, Artificial Sequence
  Synthetic
atctatatgatcatgacagaatgctggaactccaacccttagggaaccc

SEQ ID NO: 133 - DNA, 51 bp, Artificial Sequence
  Synthetic
  N_region (14)..(20): n is any of a, c, g, or t
  misc_feature (24)..(30): n is a, c, g, or t
gtgccagcaagatccaatctagannnnnnnagccgctttgggtggctccag

SEQ ID NO: 134 - DNA, 40 bp, Artificial Sequence
  Synthetic
tgaagcctctccagccaggggtccaacccttagggaaccc

SEQ ID NO: 135 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnntttctttgtggacatctgatcatggacatg

SEQ ID NO: 136 - DNA, 47 bp, Artificial Sequence
  Synthetic
atcctgcaagccctggggaagtcctacctccaacccttagggaaccc

SEQ ID NO: 137 - DNA, 59 bp, Artificial Sequence
  Synthetic
  N_region (14)..(20): n is any of a, c, g, or t
  misc_feature (24)..(30): n is a, c, g, or t
gtgccagcaagatccaatctagannnnnnncggaagctctgccggagagactatctcag

SEQ ID NO: 138 - DNA, 44 bp, Artificial Sequence
  Synthetic
gctttttgggcaagacggtctctgctccaacccttagggaaccc

SEQ ID NO: 139 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnnggtggagagacttcctgggtcaccttg

SEQ ID NO: 140 - DNA, 41 bp, Artificial Sequence
  Synthetic
gacgcagcctaccactgcaccgtccaacccttagggaaccc

SEQ ID NO: 141 - DNA, 59 bp, Artificial Sequence
  Synthetic
  N_region (14)..(20): n is any of a, c, g, or t
  misc_feature (24)..(30): n is a, c, g, or t
gtgccagcaagatccaatctagannnnnnncttacgctgcacttccatcccacggtcag

SEQ ID NO: 142 - DNA, 47 bp, Artificial Sequence
  Synthetic
gagcagcatccagttggacttccccgaatccaacccttagggaaccc

SEQ ID NO: 143 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnncaacactgacatcctcgaggtaccccag

SEQ ID NO: 144 - DNA, 40 bp, Artificial Sequence
  Synthetic
acgctgaagcggaggggcatttccaacccttagggaaccc

SEQ ID NO: 145 - DNA, 63 bp, Artificial Sequence
  Synthetic
  N_region (14)..(20): n is any of a, c, g, or t
  misc_feature (24)..(30): n is a, c, g, or t
gtgccagcaagatccaatctagannnnnnntcccctgagcctcagcacctgcttgtttggaag

SEQ ID NO: 146 - DNA, 57 bp, Artificial Sequence
  Synthetic
gggtattgaatgtgacatccgtatccagcttcctgttgtccaacccttagggaaccc

SEQ ID NO: 147 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnntacaaggagtccagaaatgctttccgcaag

SEQ ID NO: 148 - DNA, 49 bp, Artificial Sequence
  Synthetic
gccctttatggtacaacctcagaaacagcatccaacccttagggaaccc

SEQ ID NO: 149 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnttcttcatgagggaatctaagactttgggg

SEQ ID NO: 150 - DNA, 49 bp, Artificial Sequence
  Synthetic
gctgtccagattatgaatgggctcttccactccaacccttagggaaccc

SEQ ID NO: 151 - DNA, 66 bp, Artificial Sequence
  Synthetic
  N_region (14)..(20): n is any of a, c, g, or t
  misc_feature (24)..(30): n is a, c, g, or t
gtgccagcaagatccaatctagannnnnnnccagaatttgcagagactctagaacttattgaatct

SEQ ID NO: 152 - DNA, 52 bp, Artificial Sequence
  Synthetic
gatcctgtagcatggagtgacgttaccagtttttccaacccttagggaaccc

SEQ ID NO: 153 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnntcgggtagtggaaaaccagcagcctc

SEQ ID NO: 154 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnncccaccaccagcagcgactctg

SEQ ID NO: 155 - DNA, 43 bp, Artificial Sequence
  Synthetic
ccgcgacgatgcccctcaacgttatccaacccttagggaaccc

SEQ ID NO: 156 - DNA, 49 bp, Artificial Sequence
  Synthetic
aggaggaacaagaagatgaggaagaaatcgtccaacccttagggaaccc

SEQ ID NO: 157 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnncaggtgcccatcagaagcgacc

SEQ ID NO: 158 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnngtctattgctagtgagctcatcgaaaagag

SEQ ID NO: 159 - DNA, 49 bp, Artificial Sequence
  Synthetic
gatccccatcaagtacaaggcaatgaagaatccaacccttagggaaccc

SEQ ID NO: 160 - DNA, 39 bp, Artificial Sequence
  Synthetic
gtgccgccggatggtggtggtccaacccttagggaaccc

SEQ ID NO: 161 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnncaccccctctgccagctatctggag

SEQ ID NO: 162 - DNA, 48 bp, Artificial Sequence
  Synthetic
gtgaccccagactctgagaatgattttggtccaacccttagggaaccc

SEQ ID NO: 163 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnncctgtgcatcctcctgacccacag

SEQ ID NO: 164 - DNA, 42 bp, Artificial Sequence
  Synthetic
aggcatcccaacacgctgtcttttccaacccttagggaaccc

SEQ ID NO: 165 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnncgggcagagctcagggtgacag

SEQ ID NO: 166 - DNA, 44 bp, Artificial Sequence
  Synthetic
agagaagggcagaagtgcccacagctccaacccttagggaaccc

SEQ ID NO: 167 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnaacttactttggccagcattgaccttcaaa

SEQ ID NO: 168 - DNA, 44 bp, Artificial Sequence
  Synthetic
gtcagatggaacccaggacccatcctccaacccttagggaaccc

SEQ ID NO: 169 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnnacaccgcctcacagatcgactccag

SEQ ID NO: 170 - DNA, 42 bp, Artificial Sequence
  Synthetic
gtggccatcaaagtgattccccgtccaacccttagggaaccc

SEQ ID NO: 171 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnnactttcggccagctctccaatctgaag

SEQ ID NO: 172 - DNA, 47 bp, Artificial Sequence
  Synthetic
gtccacctgagagtgcacagtggagaactccaacccttagggaaccc

SEQ ID NO: 173 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnnacacggtggagtgccgcttctacag

SEQ ID NO: 174 - DNA, 44 bp, Artificial Sequence
  Synthetic
tttccatgtggtacacactcccccgtccaacccttagggaaccc

SEQ ID NO: 175 - DNA, 54 bp, Artificial Sequence
  Synthetic
  N_region (14)..(20): n is any of a, c, g, or t
  misc_feature (24)..(30): n is a, c, g, or t
gtgccagcaagatccaatctagannnnnnnaaagcccaacaccttcccccactg

SEQ ID NO: 176 - DNA, 49 bp, Artificial Sequence
  Synthetic
atgcctaccttaatgcctctgaaacaaccatccaacccttagggaaccc

SEQ ID NO: 177 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnncggcttcagctgtgggatattgcag

SEQ ID NO: 178 - DNA, 45 bp, Artificial Sequence
  Synthetic
ggcaggagcgcttcacctctatgacatccaacccttagggaaccc

SEQ ID NO: 179 - DNA, 57 bp, Artificial Sequence
  Synthetic
  N_region (14)..(20): n is any of a, c, g, or t
  misc_feature (24)..(30): n is a, c, g, or t
gtgccagcaagatccaatctagannnnnnnggtgattgttggtgatggagcctgtgt

SEQ ID NO: 180 - DNA, 54 bp, Artificial Sequence
  Synthetic
aaagacatgcttgctcatagtcttcagcaaggacctccaacccttagggaaccc

SEQ ID NO: 181 - DNA, 50 bp, Artificial Sequence
  Synthetic
  N_region (14)..(20): n is any of a, c, g, or t
  misc_feature (24)..(30): n is a, c, g, or t
gtgccagcaagatccaatctagannnnnnnagccgggccggcctagccag

SEQ ID NO: 182 - DNA, 40 bp, Artificial Sequence
  Synthetic
ttctgaaagccccatggcccctccaacccttagggaaccc

SEQ ID NO: 183 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnagcagagggctctggggagcag

SEQ ID NO: 184 - DNA, 46 bp, Artificial Sequence
  Synthetic
gacttcacctttgaaacctcgggggagtccaacccttagggaaccc

SEQ ID NO: 185 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnnggcaggagaagaggaacctgcaaag

SEQ ID NO: 186 - DNA, 49 bp, Artificial Sequence
  Synthetic
acatattttgttccaaaatggcatcttacctccaacccttagggaaccc

SEQ ID NO: 187 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctcaaggcaaagtactatgtgcagctcgag

SEQ ID NO: 188 - DNA, 49 bp, Artificial Sequence
  Synthetic
caactgaaaaagactgtggatgacctgcagtccaacccttagggaaccc

SEQ ID NO: 189 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnnctgctaatgggactgggccaagtgag

SEQ ID NO: 190 - DNA, 41 bp, Artificial Sequence
  Synthetic
gccctggccatgctactgcaggtccaacccttagggaaccc

SEQ ID NO: 191 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnnccaaaggattccgggagaactttgagtc

SEQ ID NO: 192 - DNA, 49 bp, Artificial Sequence
  Synthetic
catgtacacatctgttgacaccagcatccctccaacccttagggaaccc

SEQ ID NO: 193 - DNA, 61 bp, Artificial Sequence
  Synthetic
  N_region (14)..(20): n is any of a, c, g, or t
  misc_feature (24)..(30): n is a, c, g, or t
gtgccagcaagatccaatctagannnnnnncagtttctggcgcttagtgtaccacatcaag

SEQ ID NO: 194 - DNA, 43 bp, Artificial Sequence
  Synthetic
attgacggcgtggaggacatgctttccaacccttagggaaccc

SEQ ID NO: 195 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnngcacagacttcaccggcaccatcaa

SEQ ID NO: 196 - DNA, 49 bp, Artificial Sequence
  Synthetic
gctgctgaatgaaaattcatatgtccctcgtccaacccttagggaaccc

SEQ ID NO: 197 - DNA, 53 bp, Artificial Sequence
  Synthetic
  N_region (14)..(20): n is any of a, c, g, or t
  misc_feature (24)..(30): n is a, c, g, or t
gtgccagcaagatccaatctagannnnnnngcgcacctgtgcagccttctgca

SEQ ID NO: 198 - DNA, 43 bp, Artificial Sequence
  Synthetic
ggtcactcagctgccgcaaggagctccaacccttagggaaccc

SEQ ID NO: 199 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctctaacatgtcagcgttattgtaatgcaa

SEQ ID NO: 200 - DNA, 45 bp, Artificial Sequence
  Synthetic
gtgtgaccaattcagtgaaaggaacgtccaacccttagggaaccc

SEQ ID NO: 201 - DNA, 58 bp, Artificial Sequence
  Synthetic
  N_region (14)..(20): n is any of a, c, g, or t
  misc_feature (24)..(30): n is a, c, g, or t
gtgccagcaagatccaatctagannnnnnntgtacagcctgcgtgacttgttctcgag

SEQ ID NO: 202 - DNA, 42 bp, Artificial Sequence
  Synthetic
acgacctcgtggagaagacgccgtccaacccttagggaaccc

SEQ ID NO: 203 - DNA, 55 bp, Artificial Sequence
  Synthetic
  N_region (14)..(20): n is any of a, c, g, or t
  misc_feature (24)..(30): n is a, c, g, or t
gtgccagcaagatccaatctagannnnnnngttcccattaacgccacctccaagg

SEQ ID NO: 204 - DNA, 47 bp, Artificial Sequence
  Synthetic
atgactccgatgtgacagaggtgatgtgtccaacccttagggaaccc

SEQ ID NO: 205 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnagctgtcaccgcgggactgaaa

SEQ ID NO: 206 - DNA, 47 bp, Artificial Sequence
  Synthetic
atctttgaaccaccagctccaggagaagtccaacccttagggaaccc

SEQ ID NO: 207 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnnctgcggctgtggtccagctgag

SEQ ID NO: 208 - DNA, 49 bp, Artificial Sequence
  Synthetic
atctgcaagattgtaagacagcctgtgctctccaacccttagggaaccc

SEQ ID NO: 209 - DNA, 56 bp, Artificial Sequence
  Synthetic
  N_region (14)..(20): n is any of a, c, g, or t
  misc_feature (24)..(30): n is a, c, g, or t
gtgccagcaagatccaatctagannnnnnngcaggctgtctctctgagaacccgag

SEQ ID NO: 210 - DNA, 44 bp, Artificial Sequence
  Synthetic
gaatggcgaggatcagatctgcccctccaacccttagggaaccc

SEQ ID NO: 211 - DNA, 52 bp, Artificial Sequence
  Synthetic
  N_region (14)..(20): n is any of a, c, g, or t
  misc_feature (24)..(30): n is a, c, g, or t
gtgccagcaagatccaatctagannnnnnngccgaggcctggggtagagcag

SEQ ID NO: 212 - DNA, 47 bp, Artificial Sequence
  Synthetic
actgtggcttcacctccgagtcttaccatccaacccttagggaaccc

SEQ ID NO: 213 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnctgactttgaagtgaagacagattctacag

SEQ ID NO: 214 - DNA, 50 bp, Artificial Sequence
  Synthetic
atcacgtaaaaccaaaggaaactgaaaacactccaacccttagggaaccc

SEQ ID NO: 215 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnaagaaattatctttcctccaataaagacag

SEQ ID NO: 216 - DNA, 49 bp, Artificial Sequence
  Synthetic
atgtcatcacaatggatcccaaagacaatttccaacccttagggaaccc

SEQ ID NO: 217 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnntctggcggtattgactcttcagattcagag

SEQ ID NO: 218 - DNA, 49 bp, Artificial Sequence
  Synthetic
tctgatatcctgttgggcattctggacaactccaacccttagggaaccc

SEQ ID NO: 219 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnttctgaagactgtagttaacaagctgttca

SEQ ID NO: 220 - DNA, 60 bp, Artificial Sequence
  Synthetic
  N_region (14)..(20): n is any of a, c, g, or t
  misc_feature (24)..(30): n is a, c, g, or t
gtgccagcaagatccaatctagannnnnnnactattatttgtgatcttcagcctcaacag

SEQ ID NO: 221 - DNA, 48 bp, Artificial Sequence
  Synthetic
aattcatgcatgagacccatgatggagtctccaacccttagggaaccc

SEQ ID NO: 222 - DNA, 49 bp, Artificial Sequence
  Synthetic
gttcatacgttttatgaagctgtggggtactccaacccttagggaaccc

SEQ ID NO: 223 - DNA, 51 bp, Artificial Sequence
  Synthetic
  N_region (14)..(20): n is any of a, c, g, or t
  misc_feature (24)..(30): n is a, c, g, or t
gtgccagcaagatccaatctagannnnnnngcagaccgacggcaagttcct

SEQ ID NO: 224 - DNA, 39 bp, Artificial Sequence
  Synthetic
gctgaggccgcggaaggagctccaacccttagggaaccc

(19) United States
(12) Patent Application Publication
(10) Pub. No.: US 2022/0165355 A1
(43) Pub. Date: May 26, 2022

(54) CLASSIFICATION OF B-CELL NON-HODGKIN LYMPHOMAS

(71) Applicants: INSTITUT NATIONAL DE LA SANTE ET DE LA RECHERCHE MEDICALE(INSERM), PARIS (FR); UNIVERSITÉ DE ROUEN-NORMANDIE, MONT SAINT-AIGNAN (FR); CENTRE HENRI BECQUEREL, ROUEN (FR)

(72) Inventors: Philippe RUMINY, ROUEN (FR); Vinciane MARCHAND, ROUEN (FR); Victor BOBÉE, MONT SAINT AIGNAN (FR); Fabrice JARDIN, MONT SAINT AIGNAN (FR)

(21) Appl. No.: 17/598,775

(22) PCT Filed: Mar. 27, 2020
(86) PCT No.: PCT/EP2020/058690

§ 371 Date: Sep. 27, 2021

Related U.S. Application Data
(60) Provisional application No. 62/825,552, filed on Mar. 28, 2019.
(60) Provisional application No. 62/878,859, filed on Jul. 26, 2019.

(51) Int. Cl. G16B 25/10; C12N 15/10; C12Q 1/6886; G16B 40/20

(52) U.S. Cl. CPC G16B 25/10; C12N 15/1065; C12N 15/1096; C12Q 2600/16; G16B 40/20; C12Q 2600/112; C12Q 2600/158; C12Q 1/6886

(57) ABSTRACT

An accurate gene expression based classifier, and the associated assay, which can participate to the establishment a lymphoma diagnosis and to the evaluation of individual prognosis markers are provided. Through its use, one may distinguish subtypes of lymphomas such as ABC DLBCL, GCB DLBCL, PMBL, FL, MCL, SLL and MZL from one another.

### FIELD OF THE INVENTION

The present invention relates to assays, kits and methods for classifying B-cell Non-Hodgkin lymphomas (B-NHLs).

### BACKGROUND

B-cell Non-Hodgkin lymphomas (B-NHLs) are a highly heterogeneous group of mature B-cell malignancies that are associated with diverse clinical behaviors. Some, such as follicular lymphoma (FL), typically follow an indolent course, while others, such as diffuse large B-cell lymphoma (DLBCL), are aggressive and require intense treatment.

There are many subtypes of lymphomas, which can cause classification to be challenging. Classification is important because different types of tumors rely on the activation of different signaling pathways for proliferation and survival, and each of these pathways provides a potential site for targeted therapies. Because there is a myriad of potential different pathways for which to target treatments, obtaining an accurate diagnosis is essential if one wishes to provide patients with the most appropriate therapies.

The classification of lymphomas can be challenging, even for expert pathologists. This difficulty has recently been underscored in different studies that show that secondary reviews by hemato-pathologists who specialize in the field resulted in a change of diagnosis in up to 20% of cases with an estimated impact on care for 17% of the patients. See J. Clin. Oncol. 2017 Jun. 20; 35(18):2008-2017, Epub 2017 May 1, Impact of Expert Pathologic Review of Lymphoma Diagnosis: Study of Patients From the French Lymphopath Network.

Currently, the methods for diagnosing lymphomas are essentially based on anatomopathology: a tumor sample or a suspect tissue is removed by biopsy and analyzed under microscope. This analysis makes it possible to make a first hypotheses, based on the organization of tumor cells, their size, their shape, etc. However, this method for classifying lymphomas also requires skillful histological examination followed by immunohistochemical (IHC) analyzes to clarify the diagnosis. In France, since 2010, any biopsy concerning a lymphoma benefits from a double reading in an expert center of the national LYMPHOPATH network. Unfortunately, the risk of error in diagnosis remains high in these tumors. There is a need for solutions that will help the pathologist to reach the accurate diagnosis for these tumors.

A number of important diagnostic and prognostic markers have been identified in lymphomas, for example, MYC and BCL2 expression in DLBCLs. However, translation of the uses of these markers into clinics remains challenging. In large part, the challenge is due to the difficulty with standardizing immunohistochemistry methods.

Recently, the applicability of new quantitative RNA assays in lymphoma diagnoses have been developed. These assays provide information about the cell-of-origin (COO) classification of neoplastic cells by evaluating multiple differentiation markers or gene expression signatures associated with a prognosis. Unfortunately, none of these assays address the molecular complexity of B-NHLsNNHLsLs. Therefore, there remains a need to develop methods and assays for the classification of B-NHLs.

### REFERENCE TO TABLES SUBMITTED IN ELECTRONIC FORM

The following application contains an electronic file submitted as a text file in ASCII font entitled “database.txt” and created on Mar. 28, 2019, 882 kb. The following application also contains an electronic file submitted as a text file in ASCII font entitled “Table_IV.txt” and created on Jul. 11, 2019, 787 kb. These documents were filed with the present application as part of the pre-conversion archive. The content of each of the aforementioned electronic tables is a part of this disclosure and is incorporated by reference.

### SUMMARY OF THE INVENTION

The present invention provides pan-B lymphoma diagnostic tests that are based on a middle throughput gene expression signature, as well as methods for creating and using these tests and similar tests. The tests may be used to differentiate subtypes of cancers based on the expression of diagnostic and prognostic molecular markers (RNA markers) by the tumor cells and by bystander nontumor cells to achieve an accurate classification. These bystander cells are located proximate to the tumor cells, and may be referred to as being from the microenvironment of the tumor cells. As persons of ordinary skill in the art are aware, the microenvironment corresponds to non-tumor cells within a tumor tissue. The microenvironment participates in the survival, progression and multiplication of tumor cells. Within a microenvironment, one may find one or more if not all of fibroblasts, myofibroblasts, neuroendocrine cells, adipose cells, immune and inflammatory cells, blood and lymphatic vascular networks, and extracellular matrix (“ECM”).

In developing the present invention, the inventors combined their assay with an artificial intelligence, random forest (RF)-based algorithm. By combining gene expression profiling and machine learning, the inventors were able to increase the precise diagnosis of cancers through the integration of expression data for multiple markers that are expressed by tumor cells and their microenvironment. The contribution of the microenvironment to the molecular signature of a lymphoma is especially important when the tumor cell content is heterogeneous, which is a common problem encountered in analyses that measure gene-expression.

Various embodiments of the present invention provide a gene expression profiling assay based on a gene signature and a RT-MLPA assay. It can be more reliable than commonly used immunochemistry-assays and can be implemented in routine laboratories and used to assist pathologists in their diagnosis of these complex tumors. The assays also may be used to provide a tool for the stratification of patients in clinical trials. Further, various embodiments of the present invention may be used for determining whether a subject is eligible for a treatment. Therefore, the present invention may be used to improve the management of patients in the era of personalized medicine. The present invention may be widely adopted in the marketplace and it is not expensive.

In some embodiments, the present invention is directed to a gene expression assay kit for distinguishing subtypes of B-cell non-Hodgkin Lymphoma comprising a set of probes that is capable of distinguishing among Activated B-cell Diffuse Large B-cell Lymphoma (ABC DLBCL), Germinal Center B-cell like Diffuse Large B-cell Lymphoma (GCB DLBCL), Primary Mediastinal large B-cell Lymphoma (PMBL), Follicular Lymphoma (FL), Mantle Cell Lymphoma (MCL), Small Lymphocytic Lymphoma (SLL) and Marginal Cell Lymphoma (MZL), wherein the set of probes is capable of detecting the RNA expression of at least one marker from tumor cells of a lymphoma and at least one marker from bystander non-tumor cells located in a microenvironment of said lymphoma.

In some embodiments, the present invention is directed to a gene expression assay that is applicable to a tumor tissue sample, e.g., paraffin-embedded biopsies that are typically collected in clinical laboratories. This technology combines Reverse Transcriptase Multiplex Ligation Dependent Probe Amplification (RT-MLPA), next generation sequencing, and optionally a machine learning classifier. In some embodiments, the present invention uses the expression of diagnostic and prognostic molecular markers from tumor and non-tumor bystander cells to classify tumors into one of the seven most frequent B-cell NHL categories: ABC, DLBCL (Activated B-Cell Diffuse Large B-cell Lymphoma, also abbreviated DLBCL ABC), GCB DLBCL (Germinal Center B-cell-like Diffuse Large B-cell Lymphoma, also abbreviated DLBCL GCB or DLBCL GC), DLBCL PMBL (Primary Mediastinal (thymic) large B-cell Lymphoma, also referred to as PMBL or PMBL DLBCC), FL (Follicular Lymphoma), MCL (Mantle Cell Lymphoma), SLL (Small Lymphocytic Lymphoma), and MZL (Marginal Cell Lymphoma).

According to one embodiment, the present invention provides a method for classifying subtypes of a disease or a disorder, e.g., cancer such as lymphomas. The method comprises exposing a sample to an assay using the gene expression assay kit of the present invention and detecting the presence of expression of one or more RNA markers by the assay.

According to another embodiment, the present invention is directed to a method for classifying a lymphoma into a lymphoma subtype selected from ABC DLBCL, GCB DLBCL, PMBL, FL, MCL, SLL, and MZL. This method comprises: (a) exposing a sample to a gene expression assay, wherein the gene expression assay is capable of determining a RNA expression level of each of the following markers: TACI, CCND1, CD10, CD30, MAL, LMO2, CD5, CD23, CD28, ICOS, and CTLA4 by exposing the sample to at least one probe for each of the markers; (b) based on the expression levels determined in (a), calculating a probability that the sample belongs to each lymphoma subtype; and (c) classifying the sample as belonging to one or more of the lymphoma subtypes. Optionally, classifying may be done when the probability of belonging to ABC DLBCL, GCB DLBCL, PMBL, FL, MCL, SLL or MZL is higher than a predetermined confidence threshold. Persons of ordinary skill in the art are capable of establishing confidence thresholds. Examples of confidence thresholds are, for example, 90% or 95%. The sample may, for example, contain both tumor and non-tumor bystander cells.

According to another embodiment, the present invention is directed to a method for classifying a lymphoma into a lymphoma subtype selected from ABC DLBCL, GCB DLBCL, PMBL, FL, MCL, SLL, and MZL. This method comprises: (a) exposing a sample to a gene expression assay, wherein the gene expression assay is capable of determining a RNA expression level of each of the following markers: TACI, CCND1, CD10, CD30, MAL, LMO2, CD5, CD23, CD28, ICOS, and CTLA4 by exposing the sample to at least one probe for each of the markers; (b) based on the expression levels determined in (a), calculating a probability that the sample belongs to each lymphoma subtype; and (c) classifying the sample as belonging to one or more of the lymphoma subtypes. Optionally, classifying may be done when the probability of belonging to ABC DLBCL, GCB DLBCL, PMBL, FL, MCL, SLL or MZL is higher than a predetermined confidence threshold. Persons of ordinary skill in the art are capable of establishing confidence thresholds. Examples of confidence thresholds are, for example 90% or 95%. The sample may, for example, contain both tumor and non-tumor bystander cells.

According to another embodiment, the present invention is directed to a method for classifying a lymphoma into a lymphoma subtype selected from ABC DLBCL, GCB DLBCL, PMBL, FL, MCL, SLL, and MZL. This method comprises: (a) exposing a sample to a gene expression assay, wherein the gene expression assay is capable of determining an expression level of each of at least 137 RNA markers, wherein the 137 RNA markers are AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC (TCR-alpha), TRAF1, XBP1, XPOE571K, XPOWT, and ZAP70 by exposing the sample to at least one probe for each of the 137 RNA markers; (b) based on the expression levels determined in (a), calculating a probability that the sample belongs to each lymphoma subtype; and (c) classifying the sample as belonging to one or more of the lymphoma subtypes. Optionally, classifying may be done when the probability of belonging to ABC DLBCL, GCB DLBCL, PMBL, FL, MCL, SLL or MZL is higher than a predetermined confidence threshold. Persons of ordinary skill in the art are capable of establishing confidence thresholds. Examples of confidence thresholds are, for example 90% or 95%. The sample may, for example, contain both tumor and non-tumor bystander cells.

In this specification the name of each of the genes of interest refers to the internationally recognized name of the corresponding gene as found in internationally recognized gene sequences and protein sequences databases, including but not limited to the database from the HUGO Gene Nomenclature Committee, which is available at the following Internet address: http://www.gene.ucl.ac.uk/nomenclature/index.html, as available on 28 Mar. 2019, and which is incorporated by reference. In the present specification, the name of each of the genes of interest may also refer to the internationally recognized name of the corresponding gene, as found in the internationally recognized gene sequences database Genbank, accessible at www.ncbi.nlm.nih.gov/genebank/, as available on 28 Mar. 2019, which is incorporated by reference. Through these internationally recognized sequence databases, the nucleic acid for each of the gene of interest described herein may be retrieved by one skilled in the art.

According to another embodiment, the present invention provides a method of treating a lymphoma in a subject in need thereof, comprising: (a) classifying a lymphoma of a subject into ABC DLBCL, GCB DLBCL, PMBL, FL, MCL, SLL, and MZL by (i) determining a RNA expression level of each of a set of markers in a sample, wherein the markers within the set are CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, TACI, CD23, CD28, CD3, CD5, CD8, CXCL13, GATA3, GRB, ICOS, PD1, and TBET using a gene expression assay kit comprising or consisting of at least one probe for each of the markers within the set of markers, (ii) based on the RNA expression level for each marker, calculating for the lymphoma a probability of belonging to each of ABC DLBCL, GCB DLBCL, PMBL, FL, MCL, SLL and MZL, and (iii) classifying the lymphoma as ABC DLBCL, GCB DLBCL, PMBL, FL, MCL, SLL or MZL (optionally, classifying may be done when the probability of belonging to ABC DLBCL, GCB DLBCL, PMBL, FL, MCL, SLL or MZL is higher than a predetermined confidence threshold, e.g., 90% or 95%); and (b) treating the subject for one of the lymphomas classified in (a)(iii). For the various embodiments of the present invention, treatment may, for example, be by the administration of one or more pharmaceutical compositions or therapies such as chemotherapy or targeted therapy.

In one embodiment, the invention comprises selecting an appropriate treatment option for a subject having ABC DLBCL, GCB DLBCL, PMBL, FL, MCL, SLL or MZL (depending on the lymphoma subtype).

According to another embodiment, the present invention provides a method of treating a lymphoma in a subject in need thereof, comprising: (a) classifying a lymphoma of a subject into ABC DLBCL, GCB DLBCL, PMBL, FL, MCL, SLL and MZL by (i) determining an expression level of each of at least 137 RNA markers in a sample, wherein the at least 137 RNA markers are AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC, TRAF1, XBP1, XPOE571K, XPOWT, and ZAP70 using a gene expression assay kit comprising or consisting of at least one probe for each of the 137 RNA markers, (ii) based on the expression level calculating for the lymphoma a probability of belonging to each of ABC DLBCL, GCB DLBCL, PMBL, FL, MCL, SLL, and MZL, and (iii) classifying the lymphoma as ABC DLBCL, GCB DLBCL, PMBL, FL, MCL, SLL or MZL (optionally, classifying may be done when the probability of belonging to ABC DLBCL, GCB DLBCL, PMBL, FL, MCL, SLL, or MZL is higher than a predetermined confidence threshold); and (b) treating the subject for one of the lymphomas classified in (a)(iii).

After a lymphoma subtype is identified, a subject may be treated for that specific subtype. Treatment may, for example, be by the administration of one or more pharmaceutical compositions or therapies such as chemotherapy or targeted therapy.

According to another embodiment, the present invention is directed to an assay for classifying subtypes of a medical condition, e.g., subtypes of cancer or subtypes of a type of cancer, e.g., lymphoma. The assay may use markers that are capable of discriminating among the desired subtypes, e.g., two or more, if not all of ABC, DLBCL, GCB, DLBCL, PMBL, FL, MCL, SLL, and MZL.

In a particular embodiment, said assay kit may be in the form of a device. Assay kits may for example, be contained within kits that also comprise reagents and/or enzymes such as ligases.

In one embodiment of the assay kits of the present invention, the assay kit comprises or consists of at least one probe for, one probe for, or a pair of probes for, or is otherwise capable of detecting a marker such as an RNA marker for each of AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC (TCR-alpha), TRAF1, XBP1, XPOE571K, XPOWT, and ZAP70.

According to another embodiment, the present invention is directed to an assay kit, wherein the assay kit comprises or consist of at least one probe for, or one probe for, or a pair of probes for or is otherwise capable of detecting a marker such as an RNA marker for each of: CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, TACI, CD23, CD28, CD3, CD5, CD8, CXCL13, GATA3, GRB, ICOS, PD1, and TBET.

According to another embodiment, the present invention is directed to an assay kit, wherein the assay kit comprises or consists of at least one probe for, or one probe for, or a pair of probes for or is otherwise capable of detecting a marker such as an RNA marker for each of: TACI, CCND1, CD10, CD30, MAL, LMO2, CD5, CD23, CD28, ICOS, and CTLA4. Each probe may, for example, be an oligonucleotide such as DNA, RNA or a combination thereof.

According to another embodiment, the present invention is directed to a gene expression assay kit for distinguishing subtypes of B-cell non-Hodgkin Lymphoma comprising or consisting of a set of probes that is capable of distinguishing among ABC DLBCL, GCB DLBCL, PMBL, FL, MCL, SLL, and MZL, wherein the set of probes is capable of detecting the RNA expression of at least one marker from tumor cells of a lymphoma and at least one marker from non-tumor cells of a microenvironment of said lymphoma.

According to another embodiment, the present invention provides a gene expression assay kit for distinguishing subtypes of B-cell non-Hodgkin Lymphoma comprising or consisting of a set of probes, wherein at least seven subsets of the set of probes are capable of distinguishing among ABC DLBCL, GCB DLBCL, PMBL, FL, MCL, SLL and MZL, wherein each subset comprises or consists of one or more RNA molecules or complements thereof. Each subset may be distinct or there may be overlap among two or more subsets. Further, in some embodiments, the subsets overlap or are coextensive but when comparing any two or more of the subtypes there is at least one difference in the signature. For example, for each marker, the assay determines whether it is present or absent in a tissue sample and a classification is established by comparison to a set of profiles where each profile is defined by the combination of the presence and absence of specific markers.

According to another embodiment, the present invention provides a method for classifying a lymphoma subtype, said method comprising: (a) obtaining RNA from a lymphoma and from a microenvironment of said lymphoma; (b) exposing said RNA to a gene expression assay using the gene expression assay kit of the present invention, thereby obtaining the expression levels of said RNA; and (c) based on the expression levels of said RNA, classifying said lymphoma as a subtype selected from ABC DLBCL, GCB DLBCL, PMBL, FL, MCL, SLL, and MZL. The RNA gene expression levels can be obtained using RT-MLPA and next generation sequencing (NGS).

According to another embodiment, the present invention provides a method for developing an assay distinguishing subtypes of lymphomas, said method comprising: (a) obtaining RNA from a set of biopsy samples, wherein the set of biopsy samples comprises tissue from a plurality of lymphoma subtypes (including their microenvironments); (b) measuring the RNA expression level of at least one marker from a plurality of lymphomas and the RNA expression level of at least one marker from a microenvironment of each of the plurality of lymphomas; and (c) applying a machine learning algorithm to identify a signature of each lymphoma subtype.

According to another embodiment, the present invention is directed to a method of creating an assay. The method comprises using RT-MLPA, next generation sequencing, and machine learning classification. In some embodiments, the method comprises: (a) obtaining RNA from a set of biopsy samples, wherein the set of biopsy samples comprises tissue from a plurality of disease or disorder subtypes; (b) measuring the expression level of said RNA; and (c) applying a machine learning algorithm to classify the samples into each subtype. One may then create a plurality of probes that each alone or in combination with one or more other probes identifies markers of each subtype. Therefore, the skilled person will understand that an input variable of the machine learning algorithm is a biopsy sample and an output variable of this machine learning algorithm is the signature of a respective lymphoma subtype. Preferably, the signature of a respective lymphoma subtype is the respective lymphoma subtype from among a group of subtypes consisting of: ABC DLBCL, GCB DLBCL, PMBL, FL, MCL, SLL and MZL. The machine learning algorithm is for example the random forest algorithm. Alternatively, the machine learning algorithm is based on a neural network.

According to another embodiment, the present invention provides a method for developing an assay, said method comprising: (a) obtaining RNA from a set of biopsy samples, wherein the set of biopsy samples comprises tissue from a plurality of lymphoma subtypes; (b) measuring the RNA expression level of AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC, TRAF1, XBP1, XPOE571K, XPOWT, and ZAP70; and (c) applying a machine learning algorithm to train a classifier able to discriminate each lymphoma subtype (e.g., ABC, DLBCL, GCB, DLBCL, PMBL, FL, MCL, SLL, and MZL).

According to another embodiment, the present invention provides a method for developing an assay, said method comprising: (a) obtaining RNA from a set of biopsy samples, wherein the set of biopsy samples comprises tissue from a plurality of lymphoma subtypes; (b) measuring the RNA expression level of CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, TACI, CD23, CD28, CD3, CD5, CD8, CXCL13, GATA3, GRB, ICOS, PD1, and TBET; and (c) applying a machine learning algorithm to train a classifier able to discriminate each lymphoma subtype (e.g., ABC, DLBCL, GCB, DLBCL, PMBL, FL, MCL, SLL, and MZL).

Assay kits of the present invention may be a part of kits, and in addition to containing probes may contain solutions and reagents necessary for detection of molecules. Thus, the present invention also relates to a kit for performing the assays of the present invention. In various embodiments, for a few markers, two targets on the same gene on different exon-exon junctions are used (e.g., AID, BCL2, BCL6, MYC, CD40L), while for other targets, only a single region on the gene serves as the marker. For some immunoglobulin transcripts, some oligonucleotide probes target several markers, for example, the 5′ proche I-alpha can be incorporated into the following markers: Ialpha-Calpha, Ialpha-Cepsilon, Ialpha-Cgamma, Ialpha-Cmu. Consequently, in some embodiments more sets of probes are needed than the number of markers that are detected. By way of a non-limiting example, in one embodiment, the 224 probes of Table XVII may be used to target 137 markers, which allows discrimination when more than one marker contains a complement of a probe sequence.

Various embodiments of the present invention may serve as accurate pan-B-NHL predictors, which includes the systematic detection of numerous diagnostic and prognostic markers. These innovations may be used instead of or as a complement to conventional histology to guide the management of patients, and they may facilitate their stratification in clinical trials. For example, the invention provides a method for selecting a GCB DLBCL subject for treatment with R-CHOP therapy.

Additionally, various embodiments of the present invention are able to recognize essential B-NHLs characteristics, such as the COO gene expression signatures, together with the different contributions of the microenvironment and differentiate a variety of lymphomas in a single experiment. Thus, the present invention can prevent important clinical misclassification.

Various embodiments of the present invention may be used with routinely-fixed samples (frozen or FFPE biopsies) and require little amount of RNA. In some embodiments, a count of 100,000 reads per sample is suggested, allowing to load multiple samples in a same flow cell. The assays of the present invention can also be used in diagnostic laboratories that already use an Illumina sequencer. Interpretation of the results using gene expression histograms and the established random forest algorithm can be easily generated by persons of ordinary skill in the art.

### BRIEF DESCRIPTION OF THE FIGURES

FIGS. 1A to 1G depict data from transcriptomic expression analysis of diffuse large B-cell lymphomas. More specifically: FIG. 1A: Two-dimensional Principal Component Analysis map computed on activated B-cell (ABC) DLBCL and germinal center B-cell (GCB) DLBCL cases for 137 markers included in a panel. The expression of the 40 most discriminatory markers is plotted. FIG. 1B: Volcano plots computed on ABC DLBCL and GCB DLBCL cases for the 137 markers included in the panel showing up- or down-regulated RNA markers between these two conditions (absolute log 2-fold change >1 and a significant FDR (<0.05)). FIG. 1C: Two-dimensional Principal Component Analysis map computed on PMBL and ABC DLBCL cases for the 137 markers included in the panel. The expression of the 40 most discriminatory markers is plotted. FIG. 1D: Two-dimensional Principal Component Analysis map computed on PMBL and GCB DLBCL cases for the 137 markers included in the panel. The expression of the 40 most discriminatory markers is plotted. FIG. 1E: Volcano plots computed on PMBL and ABC DLBCL cases for the 137 markers included in the panel showing up- or downregulation between these two conditions (absolute log 2-fold change >1 and a significant FDR (<0.05)). FIG. 1F: Volcano plots computed on PMBL and GCB DLBCL cases for the 137 markers included in the panel showing up- or downregulation between these two conditions (absolute log 2-fold change >1 and a significant FDR (<0.05)). FIGS. 1G1 and 1G2: Differential expression of a selection of markers of interest that is useful for distinguishing PMBL from ABC and GCB DLBCL. **** p<10⁻⁴ and NS: not significant according to the Wilcoxon test.

FIGS. 2A to 2F depict data from differential transcriptomic analysis of diffuse large B-cell lymphoma and small cell lymphoma. More specifically: FIG. 2A: Two-dimensional Principal Component Analysis map computed on GCB DLBCL and follicular lymphoma cases for the 137 markers included in the panel. The expression of the 40 most discriminatory markers is plotted. FIG. 2B: Volcano plots computed on GCB DLBCL and follicular lymphoma cases for the 137 markers included in the panel showing up- or downregulation between these two conditions (absolute log 2-fold change >1 and a significant FDR (<0.05)). FIG. 2C: Differential expression of Tfh markers, Ki67, the macrophage marker CD68, GRB, immune escape marker PD-L2, CD40L, as well as TFH markers CD28, ICOS and GATA3 in GCB DLBCL and FL samples. **** p<10⁻⁴ by the Wilcoxon test. FIG. 2D: Two-dimensional Principal Component Analysis map computed on DLBCL and small cell lymphoma cases for the 137 markers included in the panel. The expression of the 40 most discriminatory markers is plotted. FIG. 2E: Volcano plots computed on DLBCL and small cell lymphoma cases for the 137 markers included in the panel showing up- or downregulation between these two conditions (absolute log 2-fold change >1 and a significant FDR (<0.05)). FIGS. 2F1, 2F2 and 2F3: Differential expression of a selection of markers involved in proliferation and the immune response between DLBCL and small cell lymphomas. **** p<10⁻⁴ by the Wilcoxon test.

FIGS. 3A to 3C depict data from transcriptomic expression analysis of small B-cell lymphoma. More specifically: FIG. 3A: Two-dimensional Principal Component Analysis map computed on small cell lymphoma cases, including follicular lymphoma and other small cell lymphoma cases, for the 137 markers included in the panel. The expression of the 40 most discriminatory markers is plotted. FIG. 3B: Volcano plots computed on follicular lymphoma and other small cell lymphoma cases for the 137 markers included in the panel showing up- or down-regulation between these two conditions (absolute log 2-fold change >1 and a significant FDR (<0.05)). FIGS. 3C1 and 3C2: Differential expression of a selection of GCB markers and Tfh markers in FL cases compared to other tumors, and differential expression of markers of interest among small cell lymphomas. **** p<10⁻⁴ by the Wilcoxon test.

FIGS. 4A to 4C depict data from analysis of immunoglobulin transcripts in B-NHLs. More specifically: FIG. 4A: Schematic of the regulation of immunoglobulin transcripts. Mature B-cells constitutively transcribe VDJ, Cμ and Cδ encoding IgM and IgD. In the presence of specific sets of activation signals, B-cells initiate class switch recombination through the germ line transcription of downstream Cγ, Cα, or Cε genes. The expression of sterile transcripts required for class switching after AICDA-induced genetic instability is also displayed for different subtypes. FIGS. 4B and 4C: Differential expression of the immunoglobulin transcripts IGHM and IGHD, as well as the expression of AICDA and immunoglobulin sterile transcripts required for class switching in the global cohort are plotted, showing an over expression of IGHM in tumor cells from patients with SLL, MZL, MCL, and ABC DLBCL, along with high expression of Iμ-Cμ transcript in these tumors, except for ABC DLBCL, despite AICDA expression. The sterile transcript Iε-Cε is consistently and almost exclusively expressed in FL samples.

FIGS. 5A to 5C depict data from the results of classification of the training and validation cohorts using the random forest algorithm. More specifically: FIG. 5A: Distribution of the random forest algorithm probabilities that a sample belongs to the expected class is plotted for each subtype in the training (n=283) cohort. FIG. 5B: Distribution of the random forest algorithm probabilities in the validation (n=146) cohort. FIG. 5C: Proportion of cases accurately classified by the random forest algorithm for patients with each B-NHL subtype in the training and validation cohorts. **** p<10⁻⁴ and ** p<0.01 by the Wilcoxon test.

FIGS. 6A to 6D depict progression-free survival (PFS) and overall survival (OS) in patients with DLBCL treated with rituximab plus chemotherapy from a local cohort stratified according to GCB/ABC cell-of-origin, MYC or BCL2 expression and combined MYC/BCL2 expression status determined using gene expression profiling. More specifically: survival curves for 104 patients from the local cohort stratified according to: FIG. 6A: GCB or ABC cell-of-origin determined by the random forest predictor; FIG. 6B: MYC status; FIG. 6C: for BCL2 status; or FIG. 6D: combined MYC BCL2 double expression status.

FIGS. 7A to 7C depict data from a comparison of nanostring nCounter and gene expression data. Gene expression data were compared with raw Nanostring nCounter data (Nanostring Technologies, Seattle, Wash.) obtained from 96 samples. Gene expression data were normalized to allow comparisons between individual RNA markers. Significant correlations were obtained for all 15 markers from the nCounter Lymph2Cx assay, showing a strong agreement between the two methods. Student's t test statistic and Spearman's rank correlation coefficient were used to analyze the data.

FIGS. 8A and 8B depict data from a comparison of IHC results and gene expression data. Gene expression data for the markers from the Hans algorithm (CD10, BCL6 and IRF4/MUM1), the proliferation marker Ki67 and the BCL2 and MYC prognostic markers were compared with IHC staining in 50 DLBCL samples from a clinical trial with centralized review. Significantly higher expression was observed in samples considered positive for all markers using IHC, showing that this assay represents an alternative to evaluate these markers.

FIGS. 9A and 9B depict data from transcriptomic expression of the markers from the GCB (FIGS. 9A1 and 9A2) and ABC (FIGS. 9B1 and 9B2) signatures in DLBCL. The data show differential expression of the markers from the ABC and GCB signature that is useful for distinguishing ABC from GCB DLBCL. **** p<10⁻⁴ according to the Wilcoxon test.

FIG. 10 depicts a schematic overview of a study design. Details on the clinical characteristics and pathological features of the patients are provided in Table IV, which is provided in electronic form and is incorporated into this specification in a file named Table_IV.txt.

FIG. 11 depicts data from progression-free survival (PFS) and overall survival (OS) of patients with DLBCL treated with rituximab plus chemotherapy from a local cohort stratified according to CARD11, CREB3L2, STAT6, and CD30 expression. Survival curves for 104 patients from the cohort are shown according to FIG. 11A: CARD11 status; FIG. 11B: CREB3L2 status; FIG. 11C: STAT6 status; and FIG. 11D: CD30 status.

### DETAILED DESCRIPTION

The present invention provides a new generation of RNA quantification based assays that are applicable in a routine diagnosis setting. By combining RT-MLPA with next-generation sequencing, they inform on the cellular origin of neoplastic cells through an objective and standardized evaluation of the expression of multiple differentiation markers. In some embodiments, the markers are nucleotide sequences of mRNA expressed by tumor cells, and optionally, cells from the microenvironment of the tumor cells.

In some embodiments, the present invention is directed to an accurate gene expression assay that is applicable to samples such as those derived from a formalin-fixed paraffin embedded (FFPE) sample from a subject and distinguishes the most frequent subtypes of B-cell NHLs. The sample may, for example, be a biopsy sample. Thus, the sample may first be taken from a subject and afterwards fixed with formalin and embedded in paraffin. Protocols are known in the art or are commercially available (see Keirnan, J., Histological and Histochemical Methods: Theory and Practice, 4^{th} edition, Cold Spring Harbor Laboratory Press, 2008).

In some embodiments, the present invention is directed to distinguishing subtypes of cancers. For example, the cancer may be lymphoma, such as Peripheral T-cell Lymphoma (PTCL), Hodgkin lymphoma (HL), or non-Hodgkin lymphoma (NHL). In some embodiments, the assays permit one to distinguish among subtypes of B-NHLs.

In some embodiments, the assay kit comprises, consists essentially of, or consists of molecules capable of detecting the following set of RNA markers: MYBL1; CD10; NEK6; BCL6; SERPINA9; CD86; ASB13; BCL6#2; XPOWT; MAML3; LMO2; CD22; K167; S1PR2; DUSP22; CD40; CRBN; MS4A1; CXCR5; CD28; BAFF; CD3; GATA3; CD8; PRF; MYD88e3-e4; PDL1; AID#2; CCR7; AID#1; FOXP1; CYB5R2; CREB3L2; RAB7L1; MYD88L265P; PIM2; CCND2; TACI; IRF4; and LIMD1.

In some embodiments, the assay kit comprises, consists essentially of, or consists of molecules capable of detecting the following set of RNA markers: LMO2; NEK6; IL4I1; CD95; S1PR2; TRAF1; MAML3; CD23; ASB13; PDL2; MAL; BAFF; CCND1; CD3; CD28, TCRβ; BCL2#1; CREB3L2; FOXP1; TACI; IRF4; PIM2; LIMD1; MYC#1; BANK; CD80; CCND2; CD22; RAB7L1; CXCR5; MYD88e3-e4; CYB5R2; CCR7; CCR4; CD71; AID#2; PDL1; AID#1; CD40; and MS4A1.

In some embodiments, the assay kit comprises, consists essentially of, or consists of molecules capable of detecting the following set of RNA markers: IL4I1; PDL2; CD23; PDL1; TRAF1; MAL; ALK; CD95; BAFF; CCND1; PRF; GRB; TBET; CD8; CCND2; CTLA4; CD3; GATA3; CD5; CD28; ICOS; FOXP3; TCRβ; CD27; FOXP1; CRBN; TCL1A; MYBL1; CD10; CD22; CD19; BCL6#1; CXCR5; XPOWT; CD40; KI67; BCL6#2; MS4A1; DUSP22; and NEK6.

In some embodiments, the assay kit comprises, consists essentially of, or consists of molecules capable of detecting the following set of RNA markers: BAFF; CD4; CCND1; GRB; PRF; CD8; CCND2; CD5; CD3; GATA3; CTLA4; CD40L#1; CD28; ICOS; CCR4; CD23; FOXP1; MS4A1; CRBN; CD86; CD40; BCMA; CD10; TCL1A; MYC#2; CD22; MYBL1; XPOWT; KI67; BCL6#2; BCL6#1; CD38; NEK6; CD80; FGFR1; S1PR2; APRIL; PDL1; PDL2 and CD68.

In some embodiments, the assay kit comprises, consists essentially of, or consists of molecules capable of detecting the following set of RNA markers: BCL6#2; S1PR2; CD68; BAFF; CD3; CD28; GATA3; TCRβ; ZAP70; BCL2#1; IGHM; Iμ-Cμ; CD5; CCDC50; SH3BP5; Iγ-Cγ; FOXP1; CCND2; LIMD1; BANK; CREB3L2; TACI; CCR7; CD80; IRF4; PIM2; MYD88e3-e4; CXCR5; CYB5R2; MYC#1; XPOWT; RAB7L1; PDL1; MS4A1; GD71; AID#1; AID#2; CD40; LMO2; and KI67.

In some embodiments, the assay kit comprises, consists essentially of, or consists of molecules capable of detecting the following set of RNA markers: CD86; BCL6#1; MYBL1; CD10; LMO2; ICOS; CD28; GATA3; CD4; PD1; CD8; ZAP70; FGFR1; MYD88e3-e4; CARD11; STAT6; Iμ-Cμ; SH3BP5; IGHD; CD80; LIMD1; IRF4; CD5; Iγ-Cγ; TACI; CCND1; CCND2; IGHM; CD19; CREB3L2; CD22; BCL2#1; CXCR5; CCDC50; DUSP22; KI67; BANK; B2M; MS4A1; and CD40.

In another embodiment, the assay kit comprises, consists essentially of, or consists of molecules capable of detecting the following set of RNA markers: of AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC, TRAF1, XBP1, XPOE571K, XPOWT, and ZAP70.

In some embodiments, the assay is capable of detecting the expression of at least DLBCL COO (GCB, ABC and PMBL signatures); at least MYC; at least BCL2; at least CCND1; at least COO and MYC; at least COO and BCL2; at least COO and CCND1; at least MYC and BCL2; at least MYC and CCND1; at least BCL2 and CCND1; at least COO, MYC and BCL2; at least COO, MYC and CCND1; at least COO, BCL2 and CCND1; at least CCND1, MYC and BCL2; or at least COO, CCND1, MYC, and BCL2. The expression may, for example, be detected by oligonucleotide probes.

In another embodiment, the assay kit comprises 224 molecules, wherein each molecules comprises, consists essentially of or consists of each of SEQ ID NO: 1 to SEQ ID NO: 224 or complements thereof or sequences that are at least 80%, at least 85%, at least 90%, or at least 95% identical to SEQ ID NO: 1 to SEQ ID NO: 224 or complements thereof. The molecules may in some embodiments be probes, e.g., DNA, RNA or a combination of DNA and RNA. Further the molecules may be single-stranded or double-stranded or part single-stranded and part double-stranded. In one embodiment, the molecules are each short hairpin RNA (shRNA), of for example, 40 to 200 or 60 to 120 nucleotides. The molecules used to detect markers may, e.g., be used in solution or attached to solid supports.

Technologies for detecting nucleotide sequences are well known to persons of ordinary skill in the art and include but are not limited to LD-RT-PCT (Ligation Dependent-Reverse Transcription-Polymerase Chain Reaction) or RT-MLPA, which is a well-known method for determining the level of expression of genes in a multiplex assay performed in one single tube. The general protocol for MLPA is described in Schouten, J. P. et al., (2002) Nucl. Acid Res. 30, e57, available on www.mplpa.com and also can be found in U.S. Pat. No. 6,955,901; each of these references is incorporated herein by reference in its entirety. In MLPA, probes are designed that hybridizes to the target nucleic acid sequences specific for the genes of interest. Each probe is actually in two parts, both of which will hybridize to the target cDNA in close proximity to each other. Each part of the probe carries the sequence for one of the PCR primers. Only when the two parts of the MLPA probe hybridize to the target DNA in close proximity to each other will the two parts be ligated together, and thus form a complete DNA template for the one pair of PCR primers used. The method is thus very sensitive. Moreover, MLPA reactions require small amount of cDNA. In contrast to e.g., FISH and BAC-arrays or even RT-MLPA, the sequences detected are small (about 60 nucleotides), and RT-MLPA is thus particularly adapted to the analysis of partially degraded RNA samples, for example obtained from formalin fixed paraffin embedded tissues. Compared to other techniques, an MLPA reaction is fast, cheap and very simple to perform, and it may be performed on equipment that is present in most molecular biology laboratories.

In some embodiments, the method of the present invention comprises the following steps: (i) preparing a cDNA sample from a tumor tissue sample; (ii) incubating the cDNA sample of step (i) with a mixture of pairs of probes specific of a target nucleic acid sequence of markers; (iii) connecting (i.e. ligating) the first probe to the second probe of the pairs of probes; (iv) amplifying the ligated probes produced at step (iii); and (v) detecting and quantifying the amplicons produced at step (iv).

The target nucleic acid sequence may consist of two segments which are substantially adjacent. As used herein, the term “substantially adjacent” is used in reference to nucleic acid molecules that are in close proximity to one another, e.g., within 20, 10, or 5 nucleotides or are immediately adjacent to each other. In some embodiments, when a pair of probes associate with a marker, the two probes are immediately adjacent to each other.

As used herein, “probe” or “oligonucleotide” refers to a sequence of a nucleic acid that is capable of selectively binding to a target nucleic acid sequence. More specifically, the term “probe” refers to an oligonucleotide designed to be or that has a region that is sufficiently complementary to a sequence of one strand of a nucleic acid that is to be probed such that the probe and nucleic acid strand will hybridize under selected stringency conditions for at least 80%, at least 85%, at least 90%, at least 95% or 100%. Typically, the probes of the present invention are chemically synthesized.

When there is pair of probes for a target, for each target there may be a first probe and a second probe. Each pair of first probes and second probes may be able to form a ligated probe after the ligation step. As used herein a “ligated probe” refers to the end product of a ligation reaction between the pair of probes. Accordingly, the probes are in a sufficient proximity to allow the 3′ end of the first probe that is brought into juxtaposition with the 5′ end of the second probe so that they may be ligated by a ligase enzyme.

The oligonucleotides may be exposed to a marker such as DNA or RNA under conditions that allow for hybridization based on complementarity. In some embodiments, each of the two probes may, for example, be 20 to 100 nucleotide long or 30 to 80 nucleotide long, and each with a gene specific region for example, 10 to 50 or 20 to 40 nucleotides long.

The hybridization molecule (two probes and target) can be exposed to a ligase that results in a complete probe that can be amplified. Thus, with these types of probes, each marker may be targeted by two probes, one of which is labeled 5′ and the other of which is labeled 3′. In some embodiments, for each mRNA that is probed there is at least one expression marker. For other embodiments, for one or more RNA markers, there is a plurality of e.g., 2 or 3 or more probe pair that target it. Further, as persons of ordinary skill in the art will realize, one may detect RNA by the use of other methodologies that rely on the ability of synthetizing complementary sequences in an assay to hybridize. Additionally, when collecting information from a sample, information about either or both of the presence or absence of one or more markers can be pertinent to identifying the subtype of lymphoma.

Persons of ordinary skill in the art will also recognize that if an assay kit contains a double-stranded probe, by convention, one may recite one strand's sequence and the complementary strand may be implied. Further, when a probe is single-stranded, one may refer to it by reference to that strand or to its complement. Finally, within the tables of the present invention, DNA sequences are recited (using T and not U), but unless otherwise explicitly stated, the probe may be made of RNA instead of DNA.

The clinical values of the assays of the present invention were validated by determining their accuracy in distinguishing an independent validation cohort with various histology profiles and its capacity to retrieve essential B-NHLs characteristics, such as the COO and MYC/BCL2 signatures of DLBCLs associated with the prognosis. Various embodiments of the present invention may participate in a better classification of B-NHLs, particularly between low-grade and high-grade lymphomas. The use of various embodiments of the present invention can also provide a better understanding of the molecular heterogeneity of FLs, particularly grade 3 cases, which frequently show distinctive genetic and immunophenotypic features reflecting the different cellular origins captured by the assays of the present invention.

In some embodiments, the present invention may be used in clinics. In the clinics, the systematic evaluation of dozens of diagnostic markers may be used to prevent important misclassifications. For example, three patients with MCLs in the cohort described in the examples were initially diagnosed with FL (two patients) and SLL (one patient). Correct diagnoses were only established at relapse, after the detection of t(11;14) translocations and high CCND1 expression. For these patients, the result of the classifier obtained at diagnosis and the observation of a very high expression of the CCND1 gene would have prompted additional testing and an earlier change in treatment.

Additionally, the assays may be used as a complement to conventional histology in clinics. If the percentage of lymphoma cells is sufficient, it may result in a significant simplification of the diagnostic procedures by reducing the number of immunostainings and facilitating the implementation of new diagnostic strategies. For example, in patients with DLBCLs, where new molecular classifications have recently been proposed, its coordinate implementation with next-generation sequencing, which requires the same platform, may significantly improve precision diagnosis.

In various embodiments, the present invention comprises a complete gene expression assay that combines RT-MLPA, and next-generation sequencing to classify B-cell lymphoma subtypes. This assay, which does not require any specific platform and can be applied to FFPE or other biopsies, can be implemented in many routine diagnostic laboratories. Various embodiments enable a more accurate and standardized diagnosis of B-cell lymphomas and, with the current development of targeted therapies, facilitate patient inclusion into prospective clinical trials.

In various embodiments, the present invention comprises a rigorous initial histological evaluation to distinguish reactive lymph nodes and other pathologies. Then, an immunohistochemical analyzes (IHC) can be carried out to distinguish B-cell Non-Hodgkin lymphomas (B-NHLs) with CD20 marker. CD20 is a specific marker of B-lymphoma from the pre-B stage to mature lymphoma. Most of B lymphomas strongly express CD20.

In some embodiments, a lymphoma is detected by measuring the presence or absence of at least one, at least two, at least three, at least four, at least five, or at least six markers from the cells of interest (which may be referred to a “cell origin” or “cell of origin”) and at least one, at least two, at least three, at least four, at least five, or at least six markers from a microenvironment.

By way of a non-limiting example, the set of markers from the cells of interest may comprise or consist of one or more, e.g., at least two, at least three, at least four, at least five, at least six or all of CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5. Additionally, or alternatively the set of markers from the microenvironment may comprise or consist of one or more, e.g., at least two, at least three, at least four, at least five, at least six or all of TACI, CD23, CD28, CD3, CD5, CD8, CXCL13, GATA3, GRB, ICOS, PD1, and TBET. The corresponding assay kit would comprise probes from each marker.

The measurement of the presence or absence of markers (e.g., expression level of RNA) will allow one to discriminate among different types of lymphomas, with each lymphoma having a marker profile that is distinct from that of the other lymphomas. Thus, the presence (in absolute terms and/or relative to other markers) or absence of one or more individual markers may be suggestive of more than one type of lymphoma; however, the assay will have enough markers such that the profiles of no two lymphomas are coextensive with respect to the presence or absence of all markers. Further in some embodiments, the profile is defined by the presence or absence of probes for at least one, at least two, or at least three of the following markers CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5 (group I); and the presence or absence of probes for at least one, at least two, or at least three of the following markers TACI, CD23, CD28, CD3, CD5, CD8, CXCL13, GATA3, GRB, ICOS, PD1, and TBET (group II markers). As persons of ordinary skill in the art will recognize, assays may be configured to detect a set of markers. However, in any sample, not all markers will be expressed, and the presence and absence of one or more markers can be part of or constitute the profiles of subtypes of lymphomas.

By way of non-limiting examples (with “+” meaning detection above a pre-determined level and “−” meaning an absence or detection below a pre-determined level):
a profile for DLBCL ABC may be
   From the cell of origin: TACI+; CCND1−; CD10−; CD30−; MAL−; LMO2−; CD5−;
   From the microenvironnement: CD23−; CD28−; ICOS−; CTLA4−
a profile for DLBCL GCB may be
   From the cell of origin: TACI−; CCND1−; CD10+; CD30−; MAL−; LMO2+; CD5−
   From the microenvironnement: CD23−; CD28−; ICOS−; CTLA4−
a profile for DLBCL PMBL may be
   From the cell of origin: TACI−; CCND1−; CD10−; CD30+; MAL+; LMO2+; CD5−
   From the microenvironnement: CD23+; CD28−; ICOS−; CTLA4−
a profile for MZL may be
   From the cell of origin: TACI+; CCND1−; CD10−; CD30−; MAL−; LMO2−; CD5−
   From the microenvironnement: CD23+; CD28+; ICOS+; CTLA4+
a profile for FL may be
   From the cell of origin: TACI−; CCND1−; CD10+; CD30−; MAL−; LMO2+; CD5−
   From the microenvironnement: CD23+; CD28+; ICOS+; CTLA4+
a profile for SLL may be
   From the cell of origin: TACI+; CCND1−; CD10−; CD30−; MAL−; LMO2−; CD5+; CD23+;
   From the microenvironnement: CD28+; ICOS+; CTLA4+
a profile for MCL may be
   From the cell of origin: TACI+; CCND1+; CD10−; CD30−; MAL−; LMO2−; CD5+
   From the microenvironnement: CD23−; CD28−; ICOS−; CTLA4−

As persons of ordinary skill in the art will recognize, a patient may have more than one type of lymphoma. Therefore, an assay may suggest no lymphoma, a specific subtype of lymphoma or a plurality of subtypes of lymphoma.

In some embodiments, the assay kit comprises or consists of probes for one or more if not all of the following additional group I markers: ASB13, BCL6e1-BCL6e2, BCL6e3-BCL6e4, CCDC50, CD71, CD95, FGFR1, FOXP1, ITPKB, RAB7L1, SERPINA9, STAT6, TRAF1, ANXA1, APRIL, B2M, BAFF, BANK, BCMA, CARD11, CCND2, CD138, CD19, CD22, CD27, CD38, CD40, CD70, MEF2B, MS4A1. Alternatively or additionally, in some embodiments, the assay kit comprises probes for one or more if not all of the following additional group II markers: ALK, CD4, CD45RO, CXCR5, FOXP3, INFg, LAGS, PRF, TCRbeta, TCRdelta, TCRgamma, CCR4, CCR7, CD40Le2-CD40Le3, CD40Le3-CD40Le4, CD56, CD80, CD86, CTLA4, DUSP22, PRDM1, TCL1A, TRAC, XBP1, and ZAP70.

Further, in some embodiments, addition to some or all of the aforementioned markers, the assay kit comprises probes for one or more if not all of the following additional markers: CRBN, Ialpha-Calpha, Ialpha-Cepsilon, Ialpha-Cgamma, Ialpha-Cmu, Iepsilon-Calpha, Iepsilon-Cepsilon, Iepsilon-Cgamma, Iepsilon-Cmu, Igamma-Calpha, Igamma-Cepsilon, Igamma-Cgamma, Igamma-Cmu, IGHD, IGHM, Imu-Calpha, Imu-Cepsilon, Imu-Cgamma, Imu-Cmu, JH-Calpha, JH-Cepsilon, JH-Cgamma, JH-Cmu, AIDe2-AIDe3, AIDe4-AIDe5, EBER1, HTLV1, CD163, CD68, KI67, BRAFV600E, IDH2R172K, IDH2R172T, MYD88e3-MYD88e4, MYD88L265P, RHOAG17V, XPOE571K, XPOWT, BCL6e1-Calpha, BCL6e1-Cepsilon, BCL6e1-Cgamma, BCL6e1-Cmu, Ialpha-BCL6e2, Iepsilon-BCL6e2, Igamma-BCL6e2, Imu-BCL6e2, and JH-BCL6e2.

### EXAMPLES

### Example 1

Table I shows data from the multivariate analysis of IPI, MYC/BCL2 dual expression and cell-of-origin in the local cohort of patients with DLBCL.

TABLE I

| | Overall Survival | | | Progression-Free Survival | | |
|---|---|---|---|---|---|---|
| Factor | HR | 95% CI | P | HR | 95% CI | P |
| MYC/BCL2 Double | 2.08 | 1.34-3.25 | <5.10-3 | 2.04 | 1.35-3.12 | <5.10-3 |
| Expressor (n = 28) vs | | | | | | |
| other (n = 107) | | | | | | |
| ABC (n = 53) vs GCB | 1.49 | 0.95-2.36 | 0.08 | 1.32 | 0.87-2.00 | 0.19 |
| (n = 51) subtype | | | | | | |
| IPI score 3-5 (n = 74) vs | 2.2 | 1.41-3.41 | <5.10-3 | 1.92 | 1.27-2.89 | <5.10-3 |
| IPI score 0-2 (n = 61) | | | | | | |

Table II provides data for clinical and biological characteristics of a cohort of patients with DLBCL stratified according to MYC/BCL2+ status.

TABLE II

| | MYC/BCL2+ | non-Double | | |
|---|---|---|---|---|
| Characteristic | Double Expressor | Expressor | p-value | statistical test |
| All | 28 | 106 | | |
| Age, years | | | | |
| Median (range) | 73 (36-87) | 64 (19-87) | | |
| ≤60 years | 4 | 46 | 0.0043 | Fisher exact test |
| >60 years | 24 | 60 | | |
| Sex | | | | |
| Female | 13 | 60 | 0.454 | X2 Yates |
| Male | 15 | 46 | | correction |
| Extra-lymphatic | | | | |
| involvement >1 | | | | |
| No | 17 | 69 | 0.835 | X2 Yates |
| Yes | 11 | 37 | | correction |
| Stage | | | | |
| I-II | 7 | 32 | 0.761 | X2 Yates |
| III-IV | 21 | 74 | | correction |
| B symptoms | | | | |
| No | 18 | 66 | 1 | X2 Yates |
| Yes | 10 | 40 | | correction |
| Bulky disease (>10 cm) | | | | |
| No | 18 | 66 | 1 | X2 Yates |
| Yes | 10 | 40 | | correction |
| Bone Marrow involvement | | | | |
| No | 22 | 94 | 0.23 | Fisher exact test |
| Yes | 6 | 12 | | |
| LDH | | | | |
| Normal | 20 | 93 | 0.044 | Fisher exact test |
| High | 8 | 13 | | |
| ECOG | | | | |
| 0-1 | 19 | 87 | 0.279 | X2 Yates |
| >2 | 8 | 19 | | correction |
| IPI | | | | |
| 0-2 | 8 | 53 | 0.07 | X2 Yates |
| 3-5 | 20 | 53 | | correction |
| Cell of Origin | | | | |
| ABC | 20 | 33 | <0.0001 | Fisher exact test |
| GCB | 8 | 43 | | |
| PMBL | 0 | 30 | | |

Table IV appears in the accompanying file Table_IV.txt, which is incorporated by reference. Table IV contains a sample list of IHC and gene expression data.

Tables III and V-IX provide an identification of significantly overexpressed RNA markers and corresponding E-values for each Volcano plot.

TABLE III
ABC DLBCL vs GCB DLBCL

| Overexpressed in ABC | E-value | Overexpressed in GCB | E-value |
|---|---|---|---|
| IRF4 | 1.51E−21 | NEK6 | 1.75E−15 |
| LIMD1 | 1.11E−17 | ASB13 | 2.27E−13 |
| FOXP1 | 9.06E−17 | MAML3 | 1.67E−12 |
| PIM2 | 2.01E−14 | S1PR2 | 3.66E−12 |
| CREB3L2 | 2.63E−13 | MYBL1 | 7.41E−10 |
| TACI | 1.68E−12 | CD10 | 9.83E−09 |
| RAB7L1 | 6.70E−12 | SERPINA9 | 9.41E−08 |
| CYB5R2 | 2.43E−10 | BCL6#1 | 1.00E−07 |
| CCND2 | 6.07E−08 | ITPKB | 7.49E−07 |
| CCDC50 | 9.51E−08 | LMO2 | 1.81E−06 |
| SH3BP5 | 2.36E−07 | BCL6#2 | 2.22E−05 |
| IGHM | 2.41E−07 | CD38 | 7.33E−05 |
| CCR7 | 5.89E−06 | FOXP3 | 7.72E−05 |
| PRDM1 | 2.25E−03 | | |
| JH-Cμ | 2.23E−02 | | |
| AID#1 | 2.31E−02 | | |
| AID#2 | 4.03E−02 | | |
| CARD11 | 4.82E−02 | | |

TABLE V
ABC DLBCL vs PMBL

| Overexpressed in ABC | E-value | Overexpressed in PMBL | E-value |
|---|---|---|---|
| FOXP1 | 5.86E−21 | BAFF | 8.74E−08 |
| PIM2 | 3.65E−15 | CCND1 | 4.19E−07 |
| TACI | 2.30E−14 | TRAF1 | 7.54E−07 |
| IGHM | 4.57E−14 | NEK6 | 9.66E−07 |
| IRF4 | 1.13E−13 | LMO2 | 3.93E−06 |
| BCL2#1 | 3.00E−13 | CD95 | 4.14E−06 |
| BCL2#2 | 4.79E−12 | IL4I1 | 1.13E−04 |
| LIMD1 | 5.51E−12 | MAML3 | 2.04E−04 |
| CREB3L2 | 3.86E−11 | JAK2 | 3.41E−04 |
| CXCR5 | 3.71E−10 | CD86 | 5.76E−04 |
| CYB5R2 | 6.62E−10 | PDL2 | 6.82E−04 |
| SH3BP5 | 9.13E−10 | S1PR2 | 1.51E−03 |
| TCL1A | 2.33E−09 | ITPKB | 2.20E−03 |
| BANK | 4.10E−09 | CD40L#1 | 5.02E−03 |
| MYC#1 | 1.91E−08 | ASB13 | 5.42E−03 |
| CARD11 | 1.38E−07 | MYBL1 | 6.43E−03 |
| RAB7L1 | 2.64E−07 | FGFR1 | 2.43E−02 |
| JH-Cμ | 4.48E−05 | | |
| CCND2 | 5.41E−05 | | |
| Iγ-Cγ | 6.91E−05 | | |
| CD71 | 1.15E−04 | | |
| MYC#2 | 4.94E−02 | | |

TABLE VI
GCB DLBCL vs PMBL

| Overexpressed in GCB | E-value | Overexpressed in PMBL | E-value |
|---|---|---|---|
| CARD11 | 3.54E−10 | BAFF | 3.12E−06 |
| CXCR5 | 2.84E−09 | PDL1 | 2.15E−05 |
| BANK | 1.98E−08 | CD95 | 9.82E−05 |
| CD27 | 2.29E−07 | TRAF1 | 1.07E−04 |
| BCL2#1 | 3.71E−07 | JAK2 | 1.20E−04 |
| TCL1A | 3.99E−07 | PDL2 | 6.70E−04 |
| CD22 | 1.03E−06 | IL4I1 | 1.26E−03 |
| SERPINA9 | 6.11E−06 | CCR7 | 2.07E−03 |
| IGHM | 3.00E−05 | | |
| CD10 | 1.02E−04 | | |
| BCL6#2 | 1.20E−03 | | |
| TACI | 3.81E−03 | | |
| JH-Cμ | 9.00E−03 | | |
| IGHD | 1.07E−02 | | |
| MEF2B | 1.37E−02 | | |
| BCL6#1 | 1.67E−02 | | |

TABLE VII
GCB DLBCL vs FL

| Overexpressed in GCB | E-value | Overexpressed in FL | E-value |
|---|---|---|---|
| CD68 | 9.65E−17 | ICOS | 2.41E−09 |
| S1PR2 | 1.24E−12 | CD40L#1 | 4.68E−09 |
| KI67 | 1.39E−12 | CD28 | 1.13E−08 |
| IL4I1 | 3.64E−06 | GATA3 | 4.10E−04 |
| MAML3 | 4.56E−06 | CXCL13 | 5.80E−03 |
| PDL2 | 5.66E−06 | | |
| CD163 | 1.47E−05 | | |
| PDL1 | 3.38E−05 | | |
| ASB13 | 1.54E−04 | | |
| MYC#1 | 3.16E−04 | | |
| CD70 | 4.05E−04 | | |
| GRB | 1.39E−03 | | |
| AID#1 | 3.00E−03 | | |

TABLE VIII
DLBCL vs Small cell lymphoma

| Overexpressed in | | Overexpressed in Small | |
|---|---|---|---|
| DLBCL | E-value | Cell Lymphoma | E-value |
| CD68 | 1.08E−46 | BANK | 8.14E−15 |
| BAFF | 2.45E−24 | CD40L#1 | 1.32E−12 |
| CD163 | 1.96E−23 | ICOS | 4.59E−10 |
| KI67 | 6.73E−20 | CRBN | 6.97E−10 |
| S1PR2 | 8.07E−19 | CD19 | 1.34E−09 |
| IL4I1 | 1.51E−18 | CD5 | 3.21E−09 |
| RAB7L1 | 2.36E−14 | CCDC50 | 9.17E−07 |
| AID#2 | 1.08E−13 | 1μ-Cμ | 4.44E−06 |
| AID#1 | 1.79E−13 | CD23 | 1.95E−03 |
| CYB5R2 | 1.51E−12 | CCND2 | 2.80E−03 |
| PRF | 2.18E−12 | IGHD | 2.99E−03 |
| CD71 | 2.50E−12 | CCND1 | 6.11E−03 |
| PIM2 | 9.41E−09 | Iγ-Cγ | 9.26E−03 |
| GRB | 2.05E−08 | | |
| PDL2 | 5.96E−08 | | |
| LMO2 | 3.73E−07 | | |
| MAML3 | 3.78E−07 | | |
| CD30 | 3.08E−05 | | |

TABLE IX
FL vs Other small cell lymphomas (SLL, MCL, MZL group)

| Overexpressed | | Overexpressed in other | |
|---|---|---|---|
| in FL | E-value | small cell lymphoma | E-value |
| LMO2 | 6.87E−08 | LIMD1 | 2.39E−16 |
| BCL6#2 | 2.63E−07 | CREB3L2 | 1.68E−12 |
| CD10 | 1.19E−06 | TACI | 1.12E−09 |
| BCL6#1 | 5.28E−06 | IGHM | 5.94E−09 |
| CD28 | 1.11E−05 | CD19 | 2.48E−08 |
| ICOS | 2.27E−05 | SH3BP5 | 1.61E−07 |
| MYBL1 | 3.36E−05 | STAT6 | 2.87E−07 |
| SERPINA9 | 5.62E−03 | Iμ-Cμ | 6.60E−07 |
| | | CCDC50 | 7.68E−07 |
| | | BANK | 4.75E−06 |
| | | IRF4 | 7.05E−06 |
| | | CARD11 | 7.41E−06 |
| | | IGHD | 1.29E−05 |
| | | Iγ-Cγ | 3.17E−05 |
| | | TBET | 4.13E−05 |
| | | CD5 | 5.86E−05 |
| | | CCND2 | 2.27E−04 |
| | | FGFR1 | 3.06E−04 |
| | | CCND1 | 2.39E−03 |
| | | FOXP1 | 3.03E−03 |
| | | CD70 | 4.94E−03 |
| | | JH-Cμ | 7.63E−03 |
| | | MYC#1 | 1.68E−02 |

Tables X-XV provide an identification of top differentially expressed RNA markers according to the two first components of PCA maps.

TABLE X
ABC DLBCL vs GCB DLBCL

| Principal Component 1 | | Principal Component 2 | |
|---|---|---|---|
| Positive | Negative | Positive | Negative |
| 1. CYB5R2 | 1. CD3 | 1. CD10 | 1. PRF |
| 2. AID#1 | 2. CD28 | 2. MYBL1 | 2. LIMD1 |
| 3. LIMD1 | 3. BAFF | 3. NEK6 | 3. GRB |
| 4. RAB7L1 | 4. CD40L#1 | 4. BCL6#1 | 4. IRF4 |
| 5. IRF4 | 5. CD4 | 5. SERPINA9 | 5. TACI |
| 6. AID#2 | 6. TCRγ | 6. CD86 | 6. CCND2 |
| 7. MYD88e3-e4 | 7. GATA3 | 7. BCL6#2 | 7. LAG3 |
| 8. PIM2 | 8. FOXP3 | 8. ASB13 | 8. PIM2 |
| 9. MS4A1 | 9. CD8 | 9. CD22 | 9. TBET |
| 10. FOXP1 | 10. CD45RO | 10. LMO2 | 10. CD8 |

TABLE XI
ABC DLBCL vs PMBL

| Principal Component 1 | | Principal Component 2 | |
|---|---|---|---|
| Positive | Negative | Positive | Negative |
| 1. CYB5R2 | 1. BAFF | 1. LMO2 | 1. TCRβ |
| 2. FOXP1 | 2. CD3 | 2. NEK6 | 2. CCDC50 |
| 3. LIMD1 | 3. CCND1 | 3. CD95 | 3. Iμ-Cμ |
| 4. CXCR5 | 4. MAML3 | 4. S1PR2 | 4. CD28 |
| 5. PIM2 | 5. NEK6 | 5. IL4I1 | 5. BCL2#1 |
| 6. CD71 | 6. CD4 | 6. TRAF1 | 6. IGHM |
| 7. IRF4 | 7. CD28 | 7. CD40 | 7. ICOS |
| 8. RAB7L1 | 8. APRIL | 8. MS4A1 | 8. FOXP1 |
| 9. MYC#1 | 9. CD8 | 9. PDL1 | 9. CD3 |
| 10. BCL2#1 | 10. S1PR2 | 10. CD23 | 10. FOXP3 |

TABLE XII
GCB DLBCL vs PMBL

| Principal Component 1 | | Principal Component 2 | |
|---|---|---|---|
| Positive | Negative | Positive | Negative |
| 1. CD10 | 1. PRF | 1. IL4I1 | 1. TCRβ |
| 2. KI67 | 2. CD3 | 2. CD23 | 2. FOXP3 |
| 3. MS4A1 | 3. BAFF | 3. PDL2 | 3. CD28 |
| 4. MYBL1 | 4. CCND2 | 4. PDL1 | 4. CD3 |
| 5. BCL6#1 | 5. TBET | 5. NEK6 | 5. TCRα |
| 6. XPOWT | 6. GRB | 6. TRAF1 | 6. CD5 |
| 7. TCL1A | 7. CD8 | 7. CD95 | 7. 1COS |
| 8. CD22 | 8. CD19 | 8. MAL | 8. GATA3 |
| 9. CRBN | 9. CCND1 | 9. ALK | 9. CD27 |
| 10. FOXP1 | 10. LAG3 | 10. S1PR2 | 10. CTLA4 |

TABLE XIII
GCB DLBCL vs FL

| Principal Component 1 | | Principal Component 2 | |
|---|---|---|---|
| Positive | Negative | Positive | Negative |
| 1. Ki67 | 1. CD3 | 1. PDL2 | 1. ICOS |
| 2. CD10 | 2. GATA3 | 2. BAFF | 2. MS4A1 |
| 3. XPOWT | 3. CD40L#1 | 3. CD68 | 3. BANK |
| 4. MYBL1 | 4. CD28 | 4. CD4 | 4. CD23 |
| 5. BCL6#1 | 5. CTLA4 | 5. PDL1 | 5. FOXP1 |
| 6. NEK6 | 6. CCND2 | 6. CCND1 | 6. CD28 |
| 7. BCMA | 7. CD5 | 7. APRIL | 7. CCDC50 |
| 8. BCL6#2 | 8. ICOS | 8. GRB | 8. CD40L#2 |
| 9. CD38 | 9. CCR4 | 9. PRF | 9. CD40L#1 |
| 10. CD22 | 10. FOXP3 | 10. FGFR1 | 10. Iε-Cε |

TABLE XIV
DLBCL vs Small cell lymphoma

| Principal Component 1 | | Principal Component 2 | |
|---|---|---|---|
| Positive | Negative | Positive | Negative |
| 1. CYB5R2 | 1. CD3 | 1. S1PR2 | 1. CD5 |
| 2. LIMD1 | 2. CD28 | 2. CD68 | 2. TCRβ |
| 3. CXCR5 | 3. BAFF | 3. LMO2 | 3. GATA3 |
| 4. PIM2 | 4. ICOS | 4. BCL6#2 | 4. Iμ-Cμ |
| 5. IRF4 | 5. GATA3 | 5. Ki67 | 5. SH3BP5 |
| 6. MYD88e3-e4 | 6. CD45RO | 6. IL4I1 | 6. ZAP70 |
| 7. RAB7L1 | 7. CD4 | 7. NEK6 | 7. IGHD |
| 8. TACI | 8. CD8 | 8. CD86 | 8. CCND2 |
| 9. MS4A1 | 9. TCRγ | 9. BCL6#1 | 9. FOXP1 |
| 10. AID#1 | 10. CD40L#1 | 10. MAML3 | 10. IGHM |

TABLE XV
FL vs Other small cell lymphoma (SLL, MCL, MZL group)

| Principal Component 1 | | Principal Component 2 | |
|---|---|---|---|
| Positive | Negative | Positive | Negative |
| 1. LIMD1 | 1. ICOS | 1. MS4A1 | 1. GATA3 |
| 2. CCND2 | 2. CD28 | 2. CD40 | 2. PD1 |
| 3. STAT6 | 3 LMO2 | 3. B2M | 3. ZAP70 |
| 4. CCND1 | 4. CD10 | 4. BANK | 4. CD8 |
| 5. Iγ-Cγ | 5. BCL6#2 | 5. DUSP22 | 5. FGFR1 |
| 6. CD80 | 6. CTLA4 | 6. CD86 | 6. CD4 |
| 7. CREB3L2 | 7. CD45RO | 7. CCDC50 | 7. TBET |
| 8. CXCR5 | 8. MYBL1 | 8. KI67 | 8. CD3 |
| 9. IGHD | 9. AID#2 | 9. CD71 | 9. CD30 |
| 10. Iμ-Cμ | 10. BCL6#1 | 10. TCL1A | 10. STAT6 |

Materials and Methods for Example 1

Patients

Five hundred and ten B-NHL biopsies were analyzed in this study, including 325 diffuse large B-cell lymphomas (DLBCL), 43 primary mediastinal B-cell lymphomas (PMBL), 55 follicular lymphomas (FL), 31 mantle cell lymphomas (MCL), 17 small lymphocytic lymphoma (SLL), 20 marginal zone lymphomas (MZL), 11 extranodal marginal zone lymphomas of mucosa-associated lymphoid tissue (MALT) and 8 lymphoplasmacytic lymphomas (LPL). Three hundred and sixty-six patients were diagnosed at a single institution (Center Henri Becquerel (CHB), Rouen, France). Additional patients were recruited from the SENIOR (n=96) (clinicaltrial.gov=NCT02128061) and RT3 (n=48) (clinicaltrial.gov=NCT03104478) clinical trials. All diagnoses were established according to the 2016 World Health Organization criteria by a panel of expert pathologist. For all patients, written consents were obtained before analysis of their biopsy samples.

RNA Extraction

For CHB biopsies, RNA was extracted from FFPE samples using the Maxwell 16 system (Promega, Manheim, Germany) or, when available, from frozen tissues using the RNA NOW kit (Biogentex, Seabrook, Tex.). For the samples from the RT3 and SENIOR trials, RNAs were extracted from FFPE biopsies using the Siemens TPS and Versant reagents kit (Siemens Health Care Diagnostics, Erlangen, Germany).

Assay Design and Data Processing

The RT-MLPSeq assay combined RT-MLPA and next-generation sequencing (NGS): see Wang J, Yang X, Chen H, Wang X, Wang X, Fang Y, et al. A high-throughput method to detect RNA profiling by integration of RT-MLPA with next generation sequencing technology. Oncotarget. 11 juill 2017; 8(28):46071-80.; 50-200 ng RNA were first converted into cDNA by reverse transcription using a M-MLV Reverse transcriptase (Invitrogen, Carlsbad, Calif.). cDNA were next incubated 1 hour at 60° C. with a mix of ligation dependent PCR oligonucleotides probes, including universal adaptor sequences and random sequences of seven nucleotides as unique molecular identifiers (UMI) in 1× SALSA MLPA buffer (MRC Holland, Amsterdam, the Netherlands), ligated using the thermostable SALSA DNA ligase (MRC Holland, Amsterdam, the Netherlands), and amplified by PCR using barcoded primers containing P5 and P7 adaptor sequences with the Q5 hotstart high fidelity master mix (NEB, Ipswich, Mass.). Amplification products were next purified using AMPure XP beads (Beckman Coulter, Brea, Calif.) and analyzed using a MiSeq sequencer (Illumina, San Diego, Calif.). Sequencing reads were de-multiplexed using the index sequences introduced during PCR amplification, aligned with the sequences of the probes and counted. All results were normalized according to the UMI sequences to avoid PCR amplification bias. Results are considered interpretable when at least 5000 different UMI were detected, corresponding to an average range of expression of 1 to 50.

Statistical Analysis

Correlations between immunohistochemical staining and gene expression levels were evaluated using the Wilcoxon rank sum test. Differences in patient characteristics were evaluated using the χ² and Fisher's exact tests. Principal Components Analyses (PCAs) were built using the PCA function of FactomineR package in R software ((http://www.r-project.org/). RNA markers that were significantly up- or downregulated between different conditions were analyzed using Welch's unequal variances t-test procedure and visualized in volcano plots, plotting the significance versus log 2-fold change on the y and x axes, respectively. Bonferroni's correction was applied to minimize the false positive rate. Fold changes were computed as the base 2 logarithm of the mean change in the expression level of each gene between the two conditions. RNA markers with an absolute log 2-fold change >1 and a significant FDR (<0.05) were plotted. Graphical representations were created using R software.

Training of the Machine Learning Algorithm

The training set was constructed using annotated B-NHL samples with one of the 7 following B-NHL subtypes: ABC DLBCL, GCB DLBCL, PMBL, FL, MCL, SLL and MZL (regrouping MZL, MALT and LPL). The random forest algorithm was next trained using the scikit-learn library (Python programming language (Python Software Foundation, https://www.python.org/) using a Gini index. The max_depth, n_estimators, and min_samples_split, which are the main parameters of the random forest algorithm, were set to 20, 10 000 and 4, respectively. The obtained prediction model, which relies on 5000 different trees outputting the most likely B-NHL subtype that was next applied to the independent validation sample set. Each sample is analyzed through 5000 different decision trees that together integrate all 137 markers.

Therefore, the skilled person will understand that training set was constructed to train the machine learning algorithm, said machine learning algorithm being therefore trained to receive biopsy samples, such as B-NHL samples, as different values of the input variable; and to deliver signatures of a respective lymphoma subtype for each sample, as different values of the output variable. Preferably, the signature of a respective lymphoma subtype is the respective lymphoma subtype from among a group of subtypes consisting of: ABC DLBCL, GCB DLBCL, PMBL, FL, MCL, SLL and MZL.

The random forest algorithm was trained as described above. Alternatively, when the machine learning algorithm is based on a neural network, the neural network is also trained using a training set of the same type of the one for the random forest algorithm.

Survival Analyses

The survival of the 104 patients with DLBCL who were treated with a combination of rituximab and chemotherapy between 2000 and 2017 at the Centre Henri Becquerel was analyzed considering a risk of 5% as a significance threshold. Overall survival (OS) was computed from the day of treatment to death from any cause or right-censored at five years or the last follow-up. Progression-free survival (PFS) was computed from the day of treatment to disease progression, relapse, or death from any cause, or right-censored at 5 years or the last follow-up. Survival rates were estimated with the Kaplan-Meier method that provides 95% CIs, and significant differences between groups were assessed using the log-rank test. Different thresholds were tested to determine the ones that led to the most significant segmentation of patients and to evaluate the prognostic value of MYC and BCL2. Those thresholds were subsequently combined to define the MYC+/BCL2+ double expression group. All analyses were performed using the Python survival package version 2.37.4.

Results

Gene Selection

A panel of 137 gene expression markers was designed for this study. The inventors purposefully included many B-cell differentiation markers identified in the WHO (Word Health Organization) classification of lymphoid neoplasms for their capacity to discriminate the main subtypes of B-cell NHLs. The inventors also selected RNA markers corresponding to the ABC, GCB and PMBL DLBCL signatures, direct therapeutic targets and different prognostic markers. The inventors included T cell and macrophage makers, along with RNA markers involved in the anti-tumor immune response to analyze the contribution of the microenvironment. Specific probes were also designed to evaluate the expression of various IGH transcripts, to detect some recurrent somatic point mutations and to evaluate the EBV and HTLV1 infection status (Tables XV and XVI).

Technical Validation

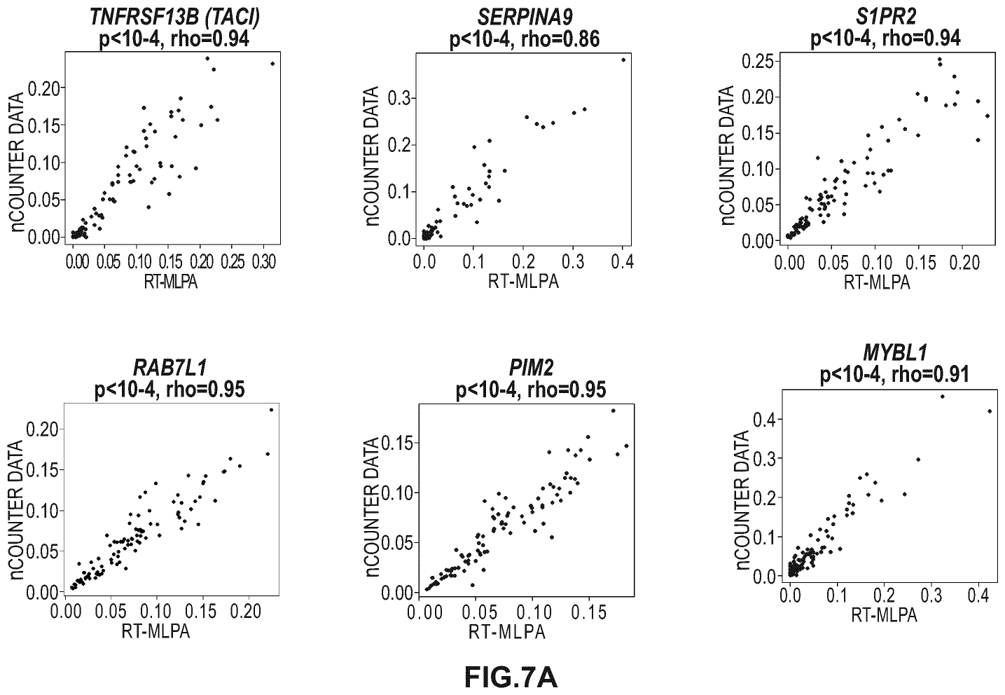

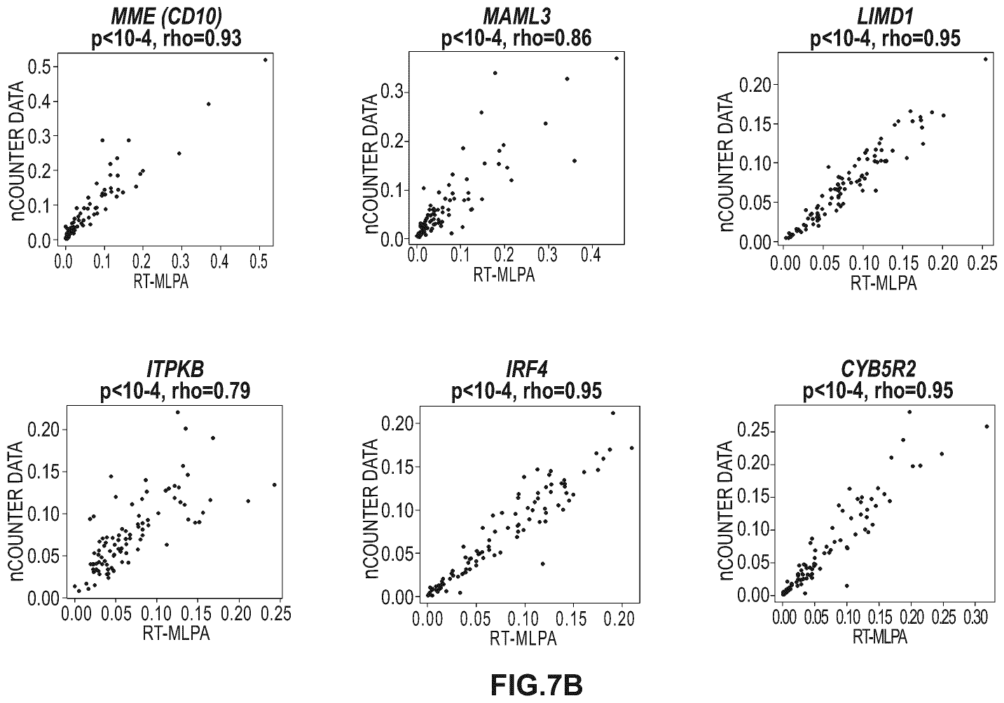

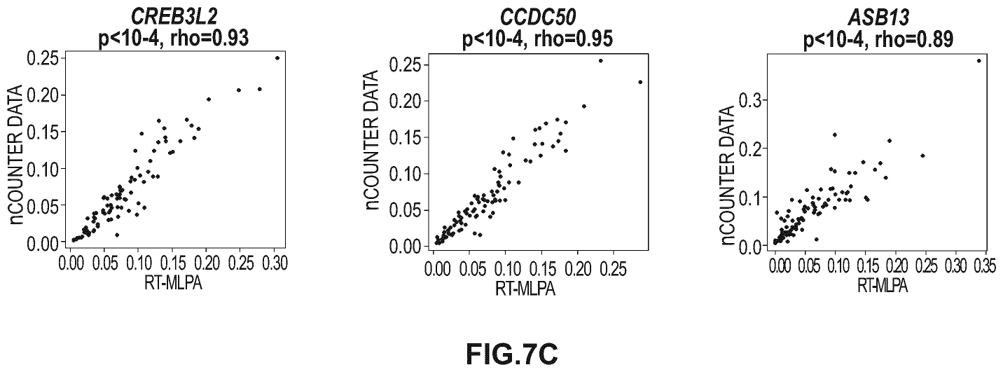

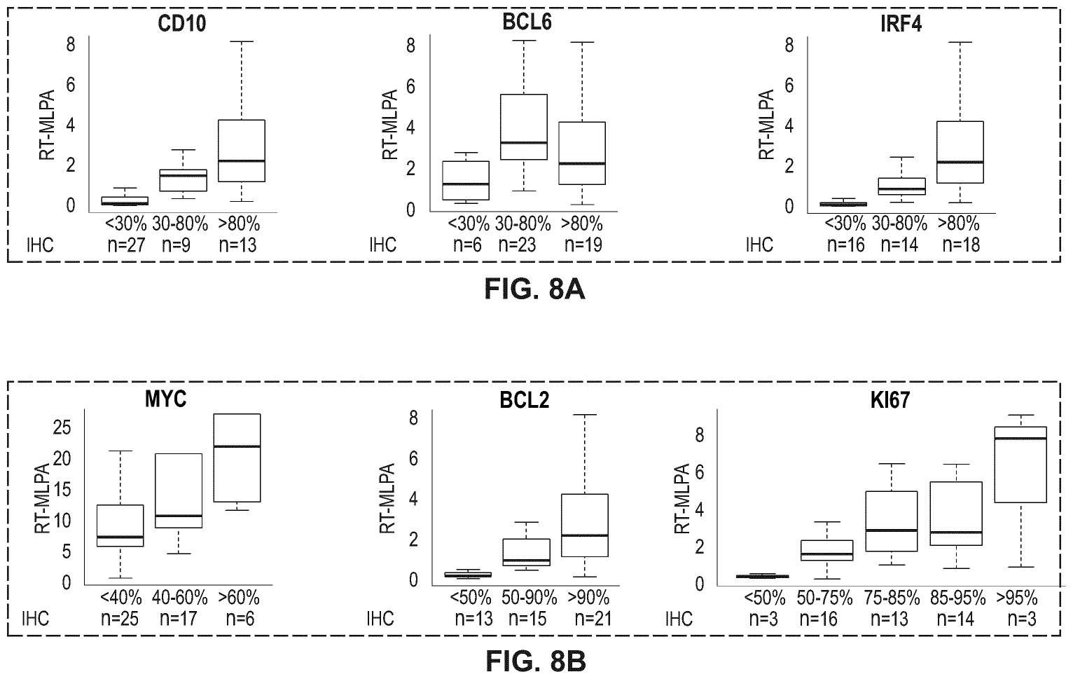

For validation, the inventors first compared the method with the Nanostring Lymph2Cx assay. As shown in FIGS. 7A, B and C, linear correlations were observed for the 15 RNA markers evaluated using the two methods applied to the 96 FFPE biopsy samples from the SENIOR clinical trial. Significant correlations with immunochemical staining was also obtained for the 48 DLBCL samples from the RT3 clinical trial (CD10, BCL6, MUM1, MYC, BCL2 and Ki67, reviewed by a panel of expert pathologists from the LYSA) (FIGS. 8A and B), indicating excellent technical concordances.

DLBCL COO Assignment

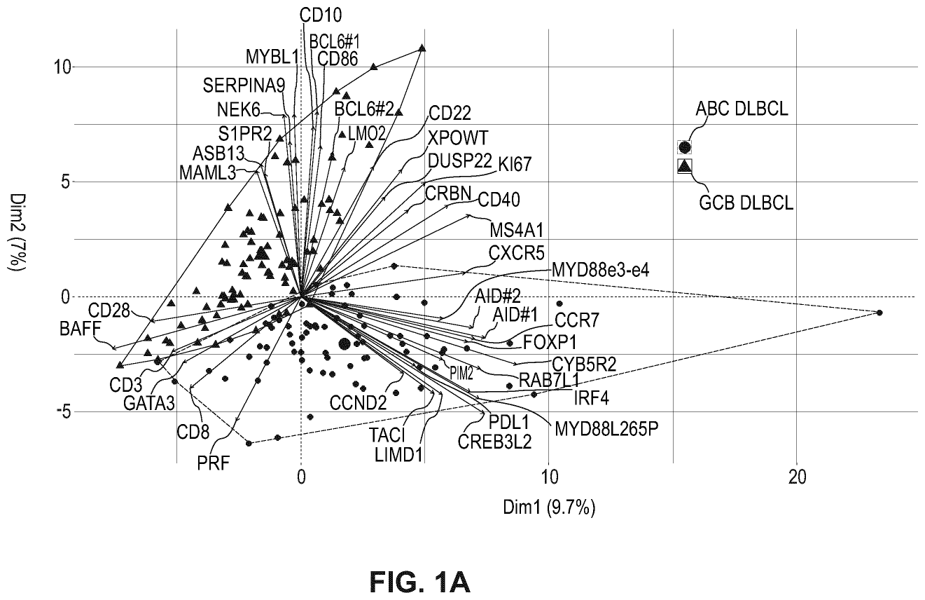

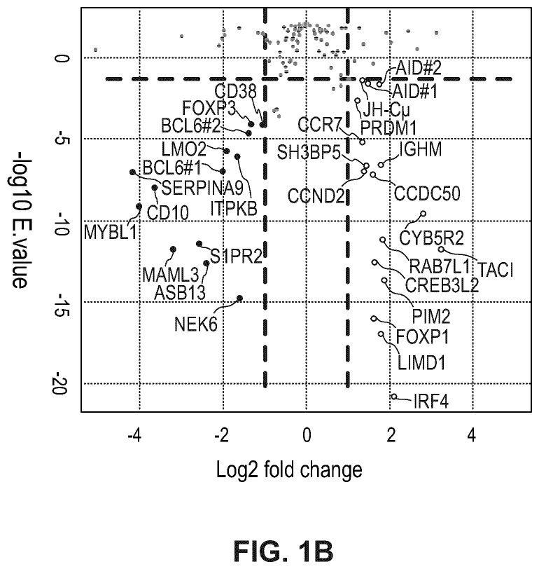

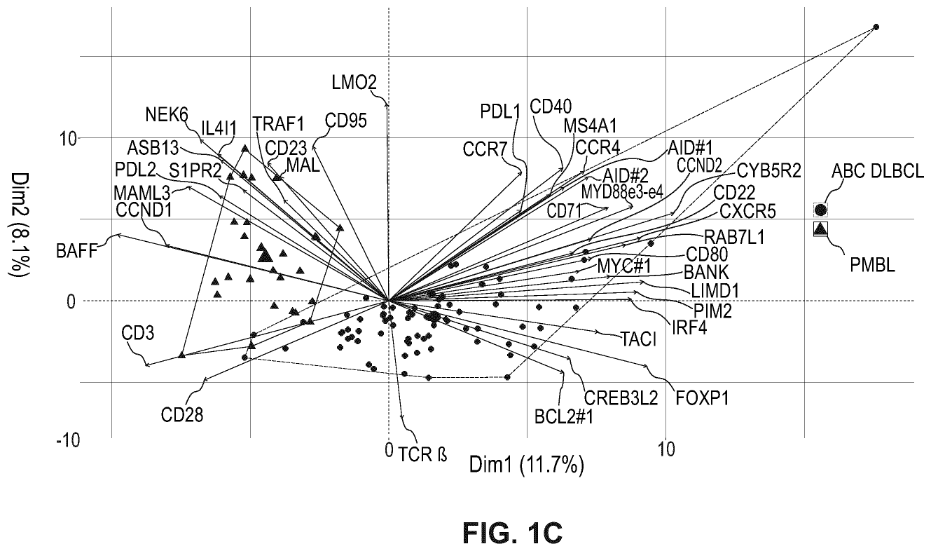

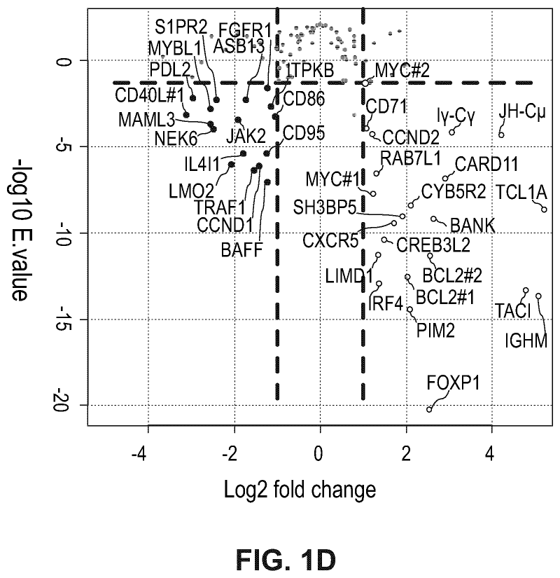

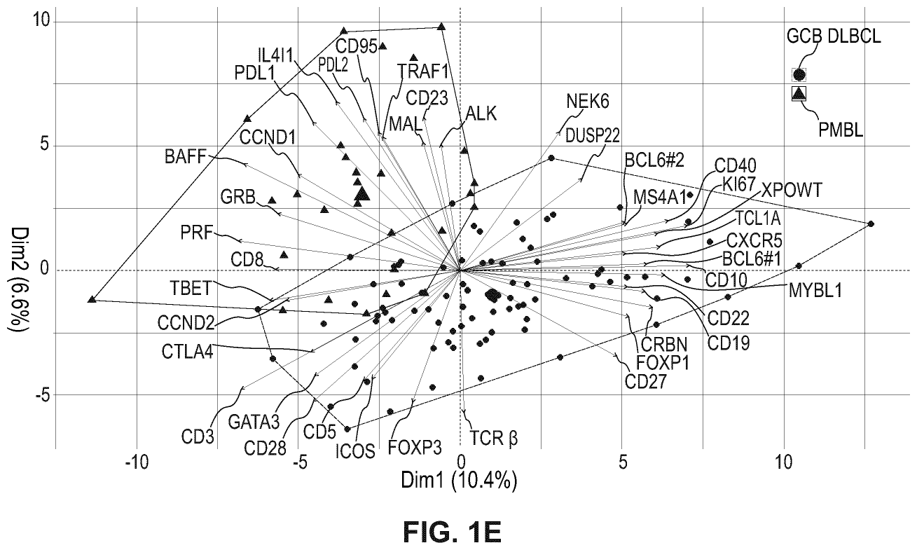

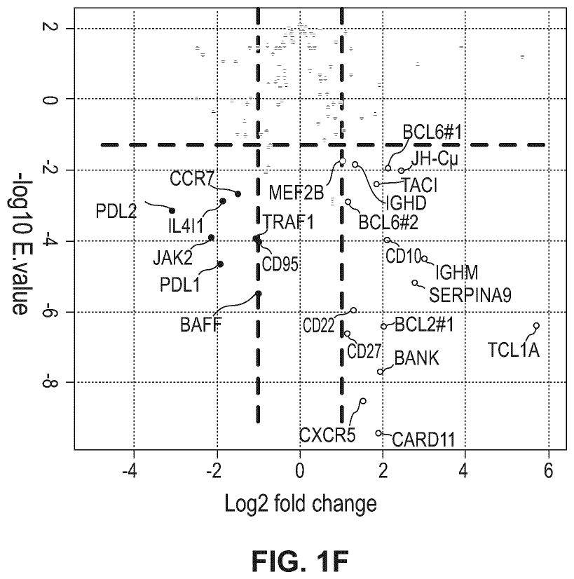

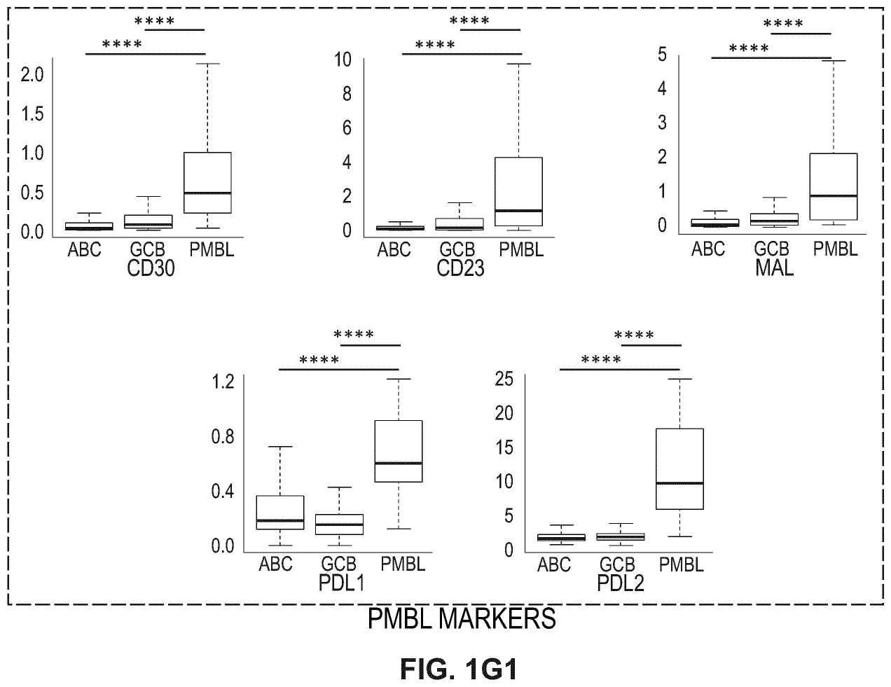

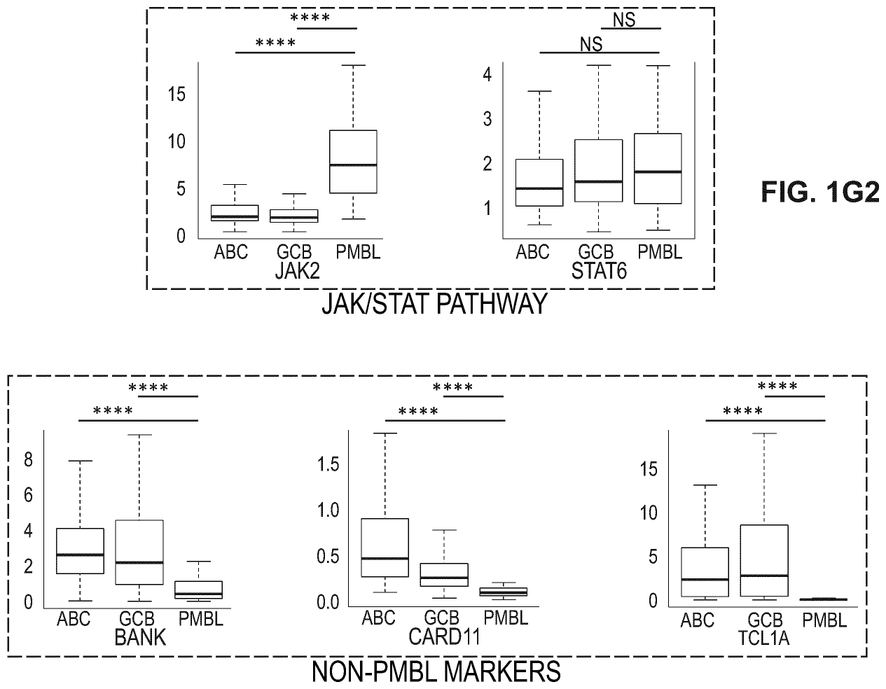

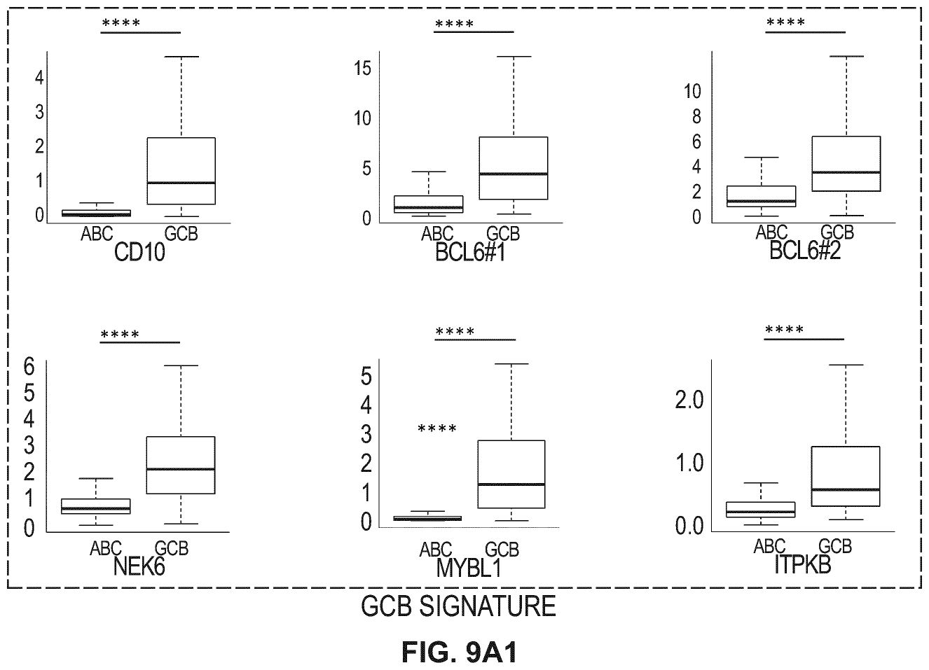

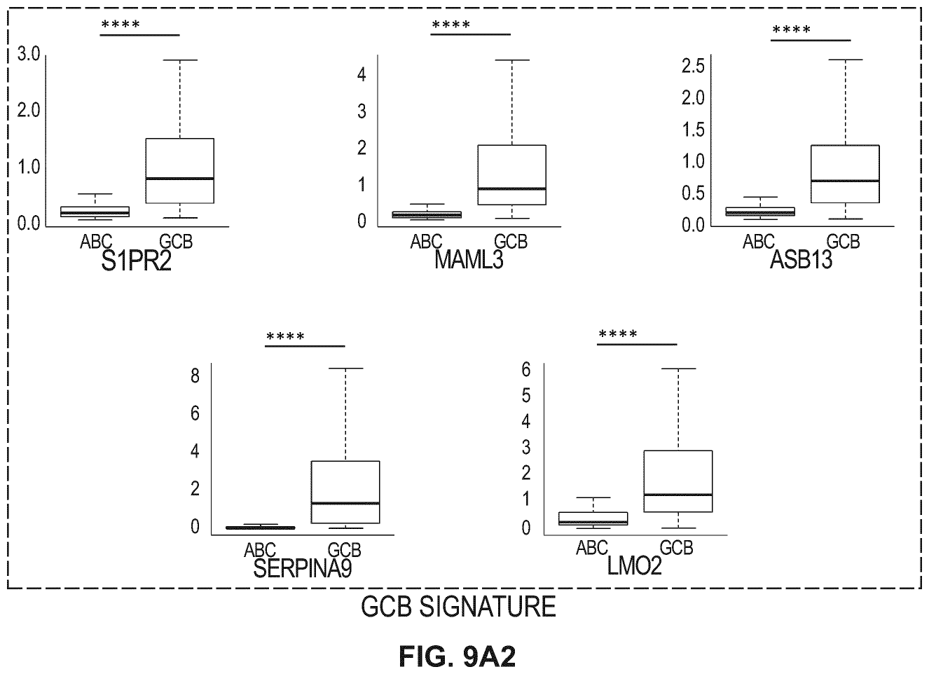

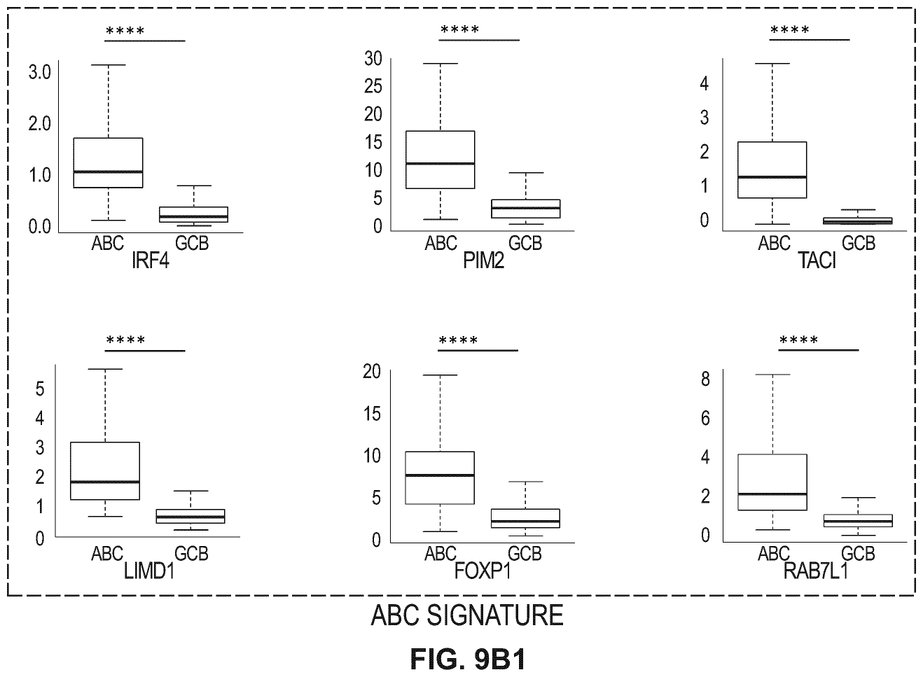

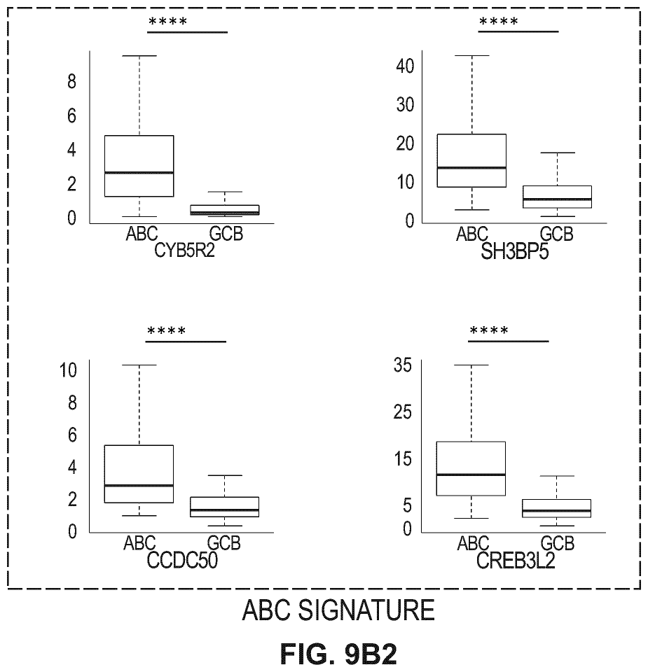

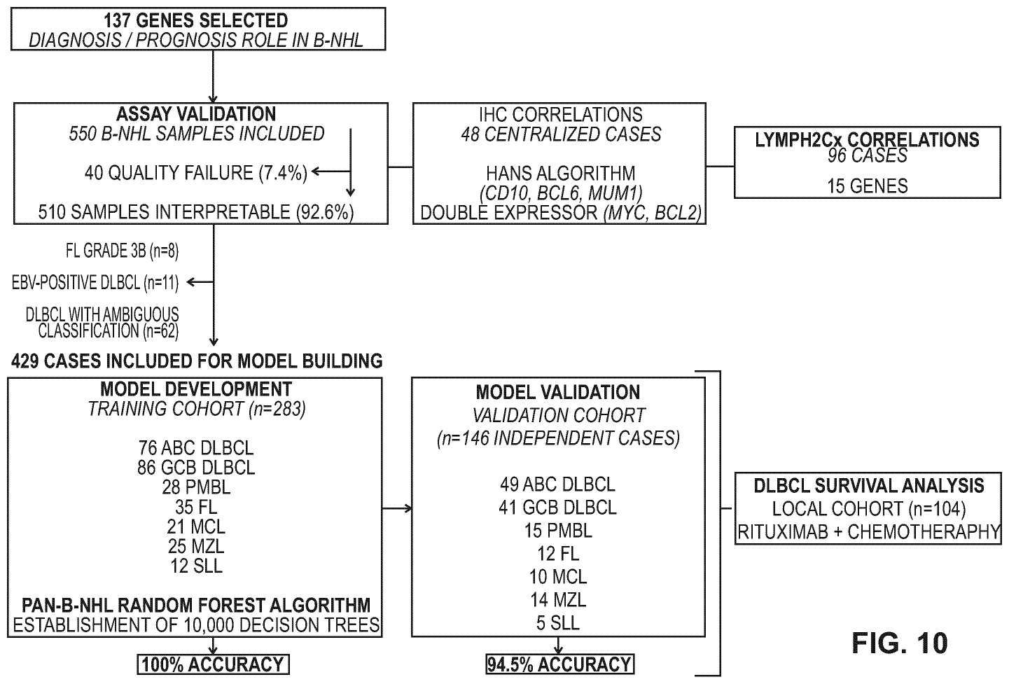

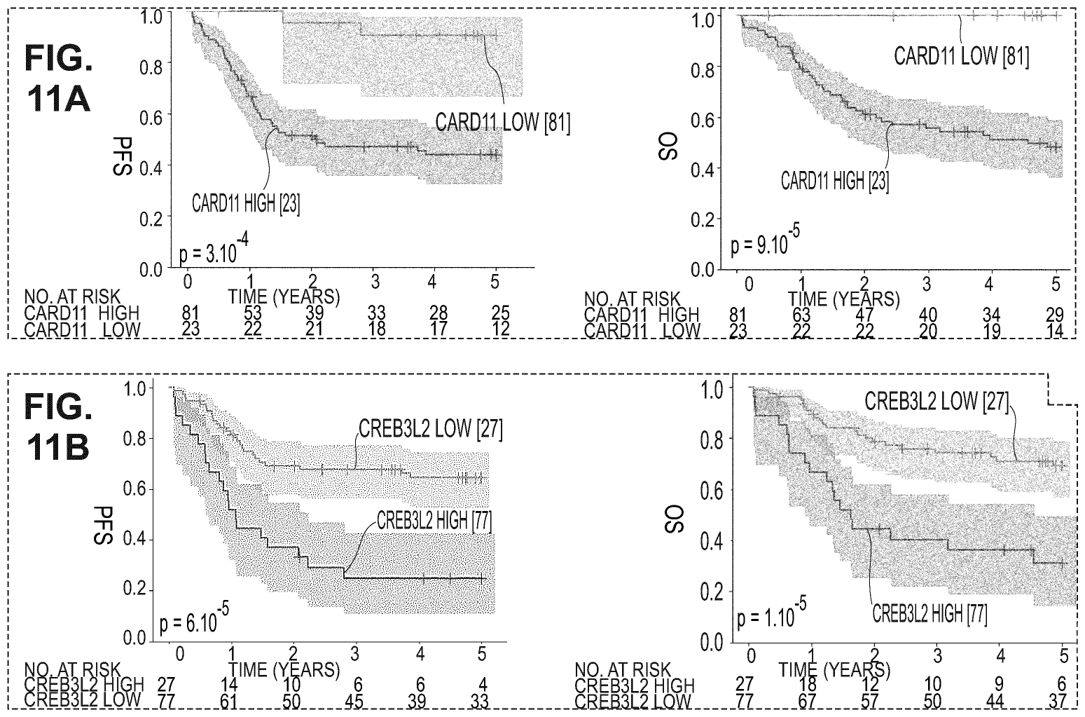

The inventors next addressed the ability of the panel of markers to discriminate the different subtypes of B-cell NHLs. The inventors first tested capacity of the assay to recapitulate the COO classification of DLBCLs. As shown in FIGS. 1A-1G, an unsupervised principal component analysis (PCA) and differential gene expression analysis (DGEA, volcano plot) of the 125 ABC and 127 GCB DLBCL cases from the cohort efficiently distinguished these two lymphoma subtypes (FIG. 1A), retrieving the expected gene expression signatures (FIG. 1B, Tables X-XV and FIG. 9). This analysis also identified a COO-independent T cell component (CD28, BAFF, CD3, GATA3, CD8, and PRF) that reflects various levels of T cell infiltration in these tumors.

The inventors next tested the capacity of the assay to discriminate PMBLs from other DLBCLs. The first components of the PMBL vs ABC and PMBL vs GCB PCA maps retrieved the three expected signatures (FIG. 1C and FIG. 1E). As shown in FIG. 1D—FIG. 1G, the results confirmed that the CD30 and CD23 markers, which are often evaluated using immunochemistry in the clinic for diagnostic purposes, were overexpressed at the RNA level in these samples. The data were also consistent with the high expression of PDL1, PDL2 and JAK2 and the downregulation of BANK, CARD11 and TCL1A reported in these tumors by Rosenwald A, Wright G, Leroy K, Yu X, Gaulard P, Gascoyne R D, et al. Molecular diagnosis of primary mediastinal B cell lymphoma identifies a clinically favorable subgroup of diffuse large B cell lymphoma related to Hodgkin lymphoma. J Exp Med. 15 sept 2003; 198(6):851-62

DLBCL/Small Cell Lymphoma Classification

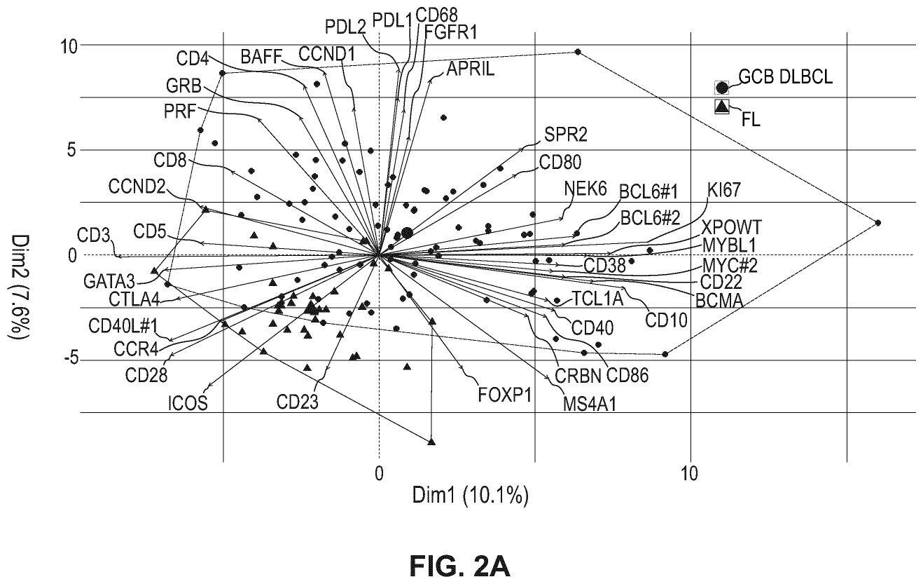

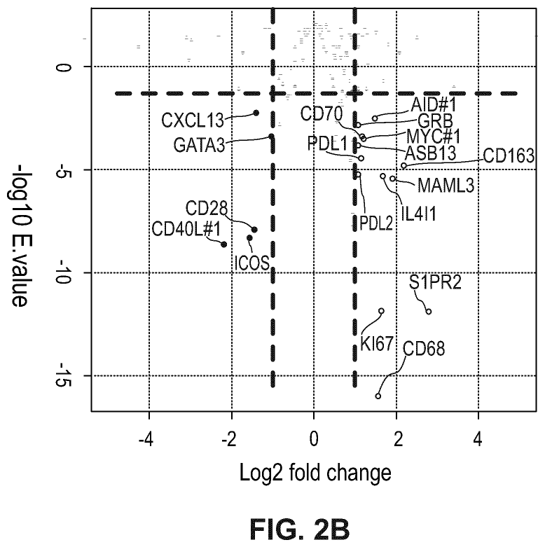

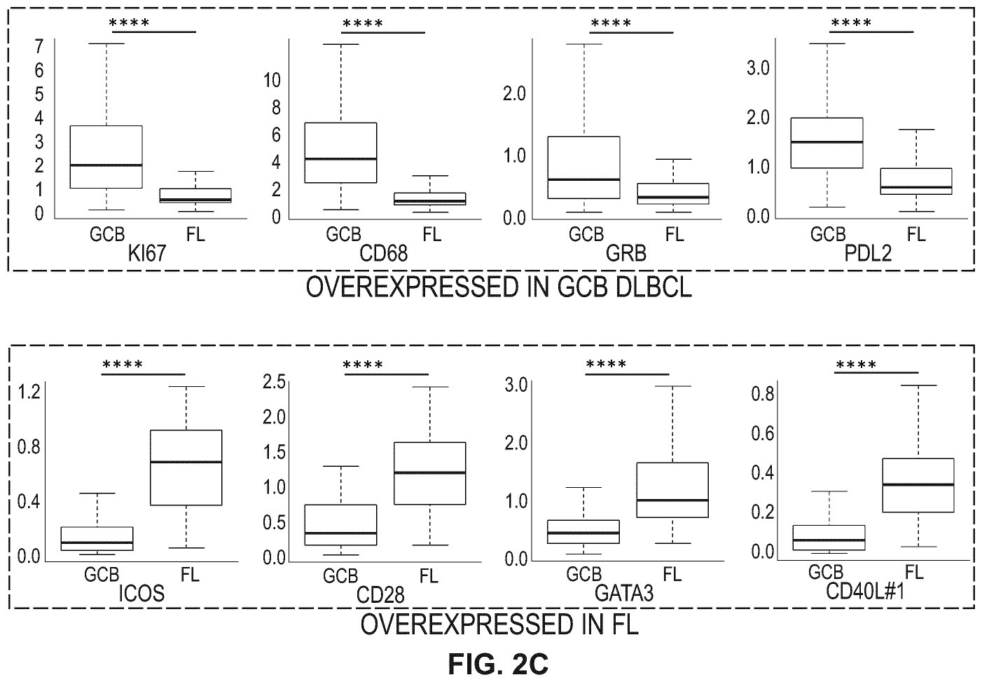

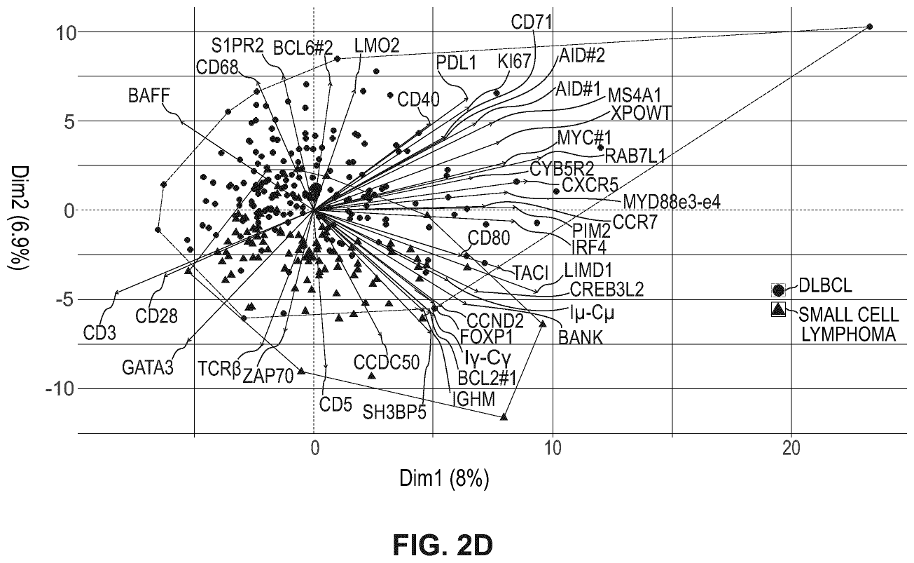

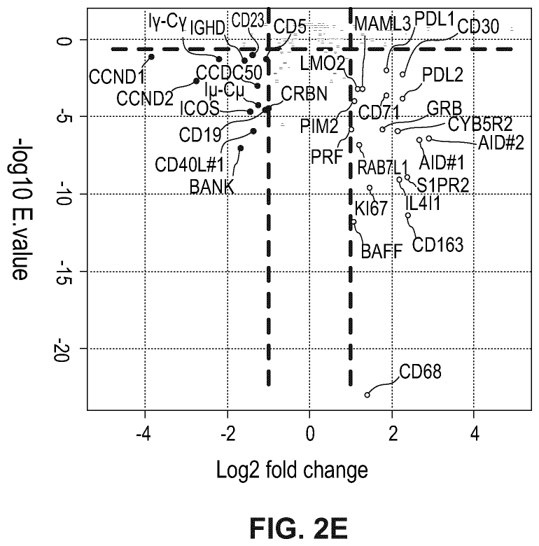

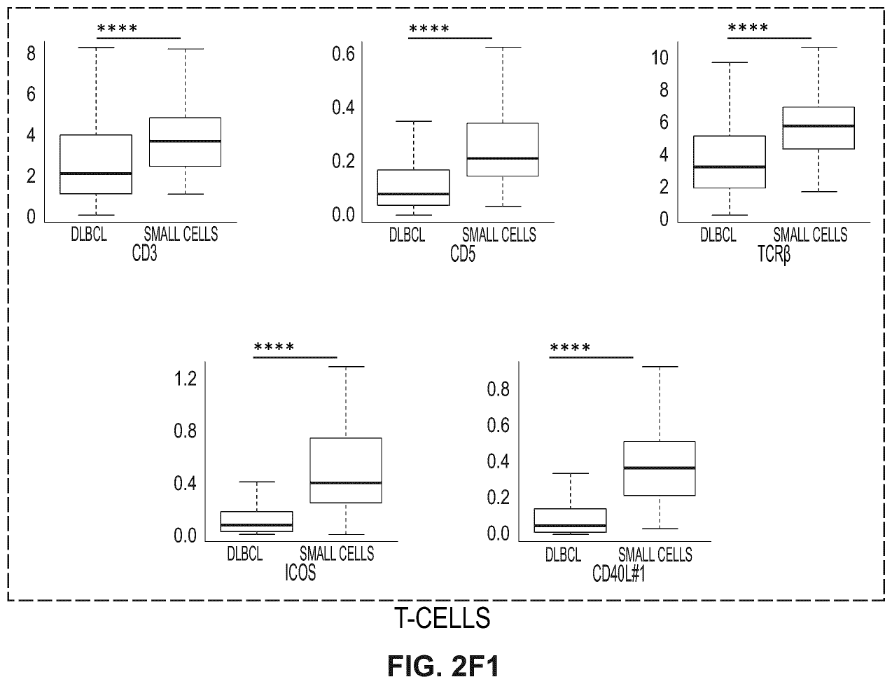

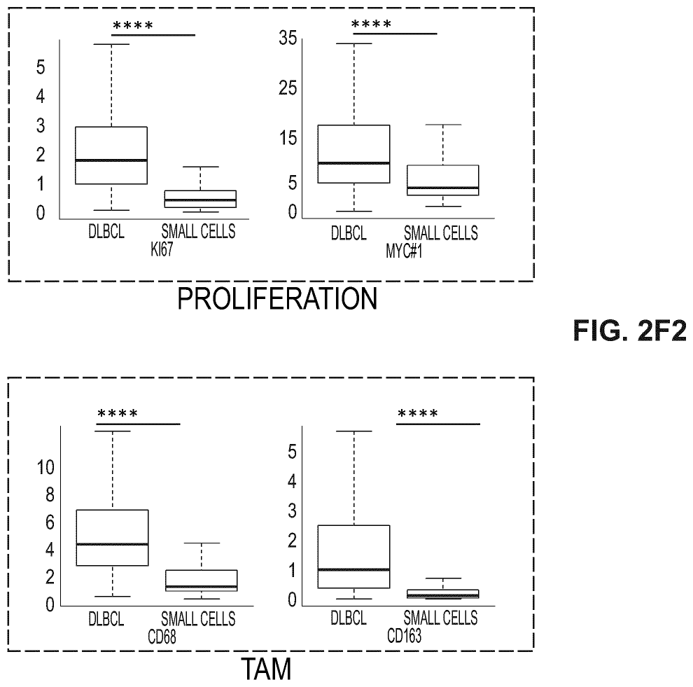

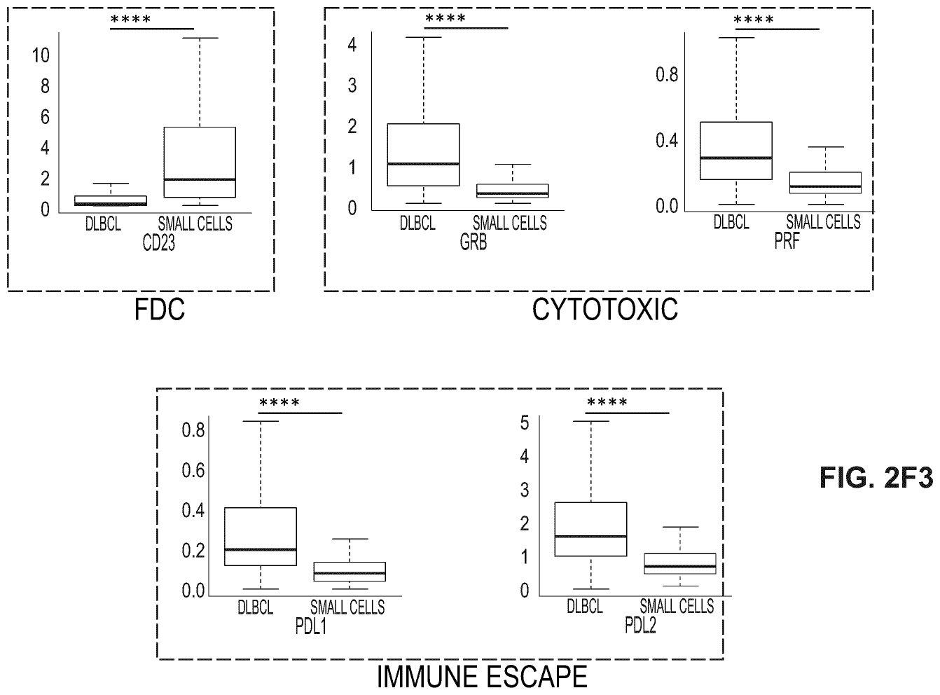

The inventors next addressed the classification ability of the markers expressed by cells in the microenvironment. The inventors first compared GCB DLBCLs and FLs, two lymphomas that develop from germinal center B-cells. As shown in FIG. 2A, the first dimensions of the PCA map identified 3 major components. The first, which is associated with GCB DLBCLs, essentially regrouped GCB markers (CD10, MYBL1, NEK6, and BCL6), reflecting the higher percentage of malignant cells in these tumors. As shown in FIGS. 2B-2C, GCB DLBCLs were also characterized by the expression of the K167 proliferation marker, the tumor-associated macrophage (TAM) marker CD68, and cytotoxic and immune escape markers (GRB, PD-L1 and PD-L2). As expected, the second component of this PCA, which is associated with FLs, regrouped many T cell markers (CD3, CD5, CD28, CTLA4, GATA3 and CCR4). FLs also significantly overexpressed CD23, due to the presence of follicular dendritic cells, as well as the Tfh markers ICOS, CD40L and CXCL13.

As shown in FIGS. 2D-2F, the same PCA and DGEA methods applied to the whole cohort of cases revealed that the high expression of KI67, germinal center-associated RNA markers (LMO2, BCL6, MAML3, S1PR2, and CD40), the CD68 and CD163 TAM markers, the GRZB and PRF cytotoxic markers, and the PD-L1 and PD-L2 immune checkpoints inhibitors were a common characteristic of aggressive lymphomas, regardless of the COO classification. This observation reflects the high turnover of lymphoma cells within these tumors, together with the presence of scavenger cells and the existence of an active anti-tumor immune response. Conversely, low-grade lymphoma were characterized by the expression of T cell markers (CD3, CD5, the beta chain of the TCR, ICOS and CD40L) and a follicular dendritic cell marker (CD23), reflect the crosstalk between lymphoma cells and their environment for survival and proliferation.

Small B-Cell Lymphoma Classification

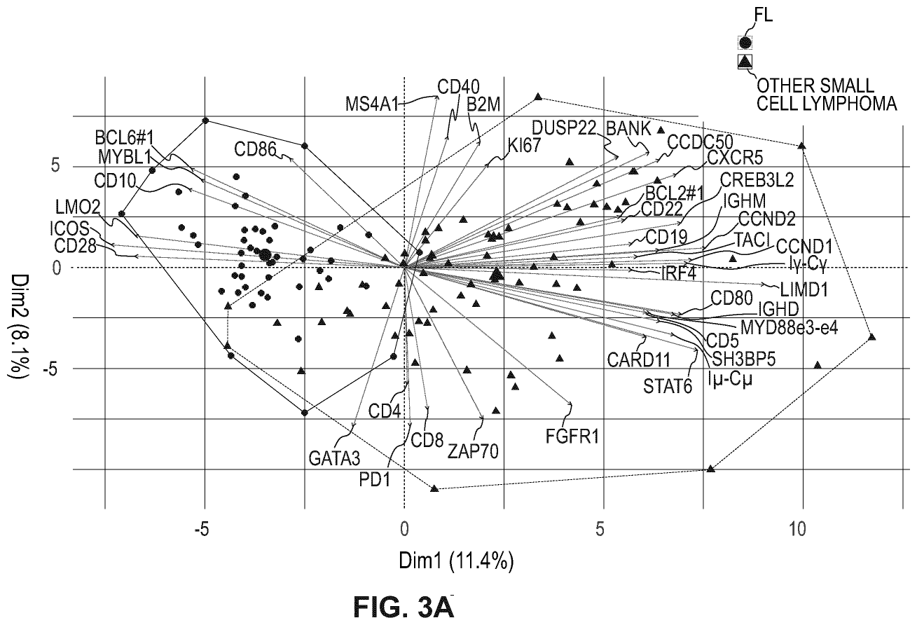

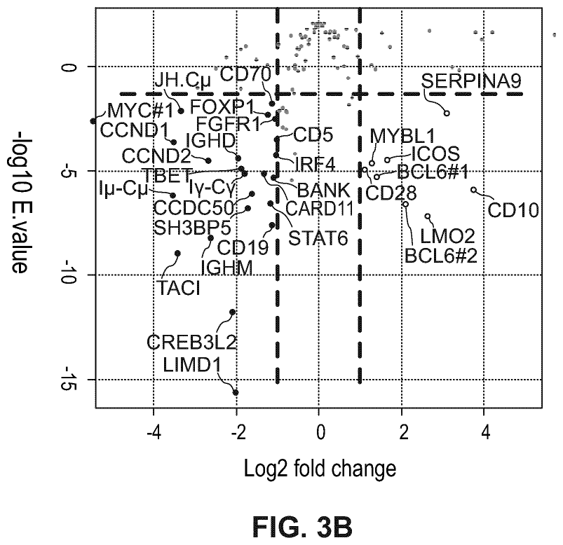

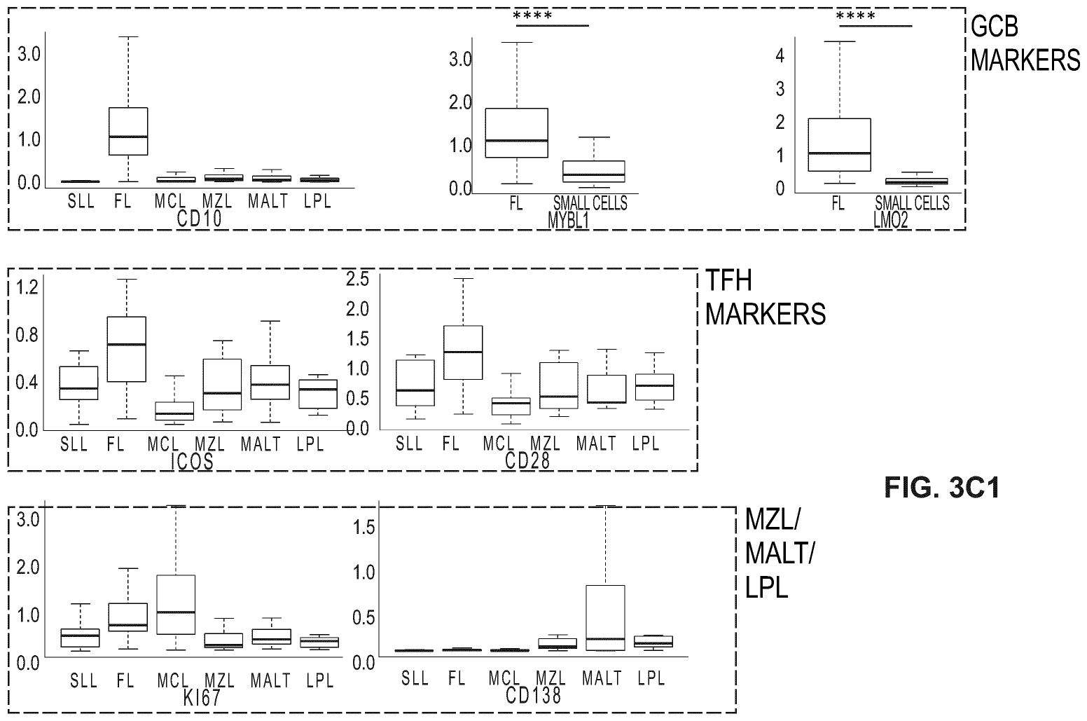

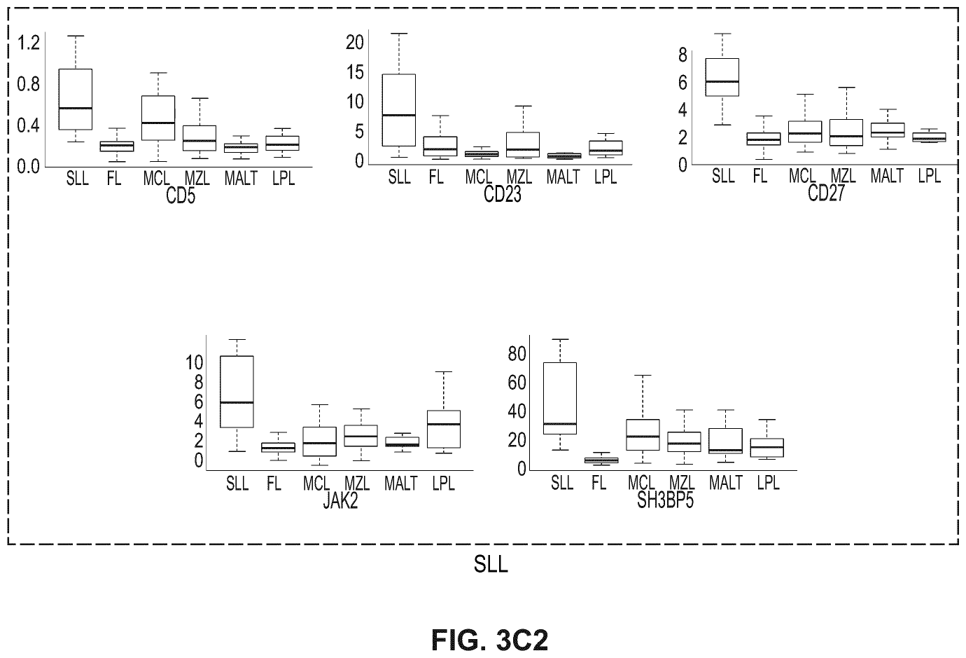

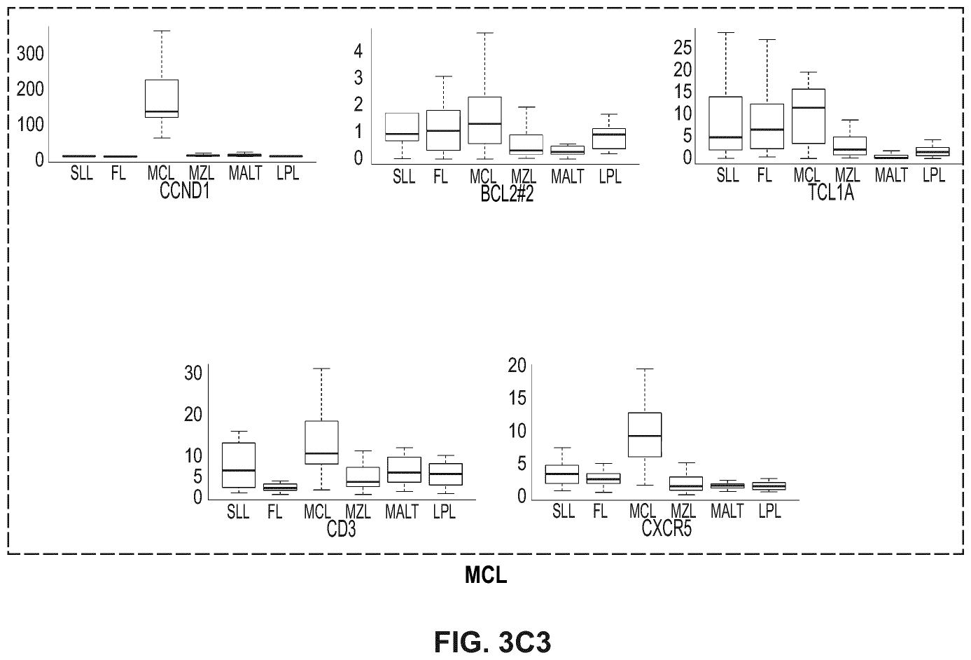

The inventors next addressed the capacity of the assay to discriminate the different subtypes of small cell B-NHLs. As shown in FIG. 3A, the first dimensions of the PCA map restricted to low grade B-NHLs identified two major components. The first, which is associated with FLs, regrouped GCB (BCL6, MYBL1, CD10 and LMO2) and T cells markers (CD28, ICOS). The second regrouped many activated B-cell markers (LIMD1, TACI, SH3BP5, CCDC50, IRF4, and FOXP1), consistent with the late GC or memory B-cell origin of others small B-cell lymphoma.

The inventors next addressed the capacity of the assay to retrieve the main characteristics used in the clinics for the classification of these tumors (FIGS. 3C1, 3C2 and 3C3). The CD5pos, CD23pos, CD10neg phenotype of SLLs was correctly identified. Interestingly, these tumors also expressed CD27, consistent with their mature B-cell origin, JAK2, suggesting the activation of the JAK/STAT pathway, and downregulated SH3BP5, indicating a possible negative regulatory effect on Bruton's tyrosine kinase activity. In MCLs, the assay retrieved the expected CCND1high, CD5high and BCL2high phenotype, together with the expected downregulation of CD10 and CD23. Interestingly, TCL1A and CCDC50, both of which are associated with survival in patients with this pathology, and the B-cell chemokine receptor CXCR5, which is involved in dissemination, were overexpressed in these tumors compared to other small B-cell NHLs. Finally, MZL showed the expected CD5pos, CD10pos, CD23neg phenotype, together with high expression of CD138 and low expression of Ki67.

IGH Transcripts Participate in the Classification of B-NHLs

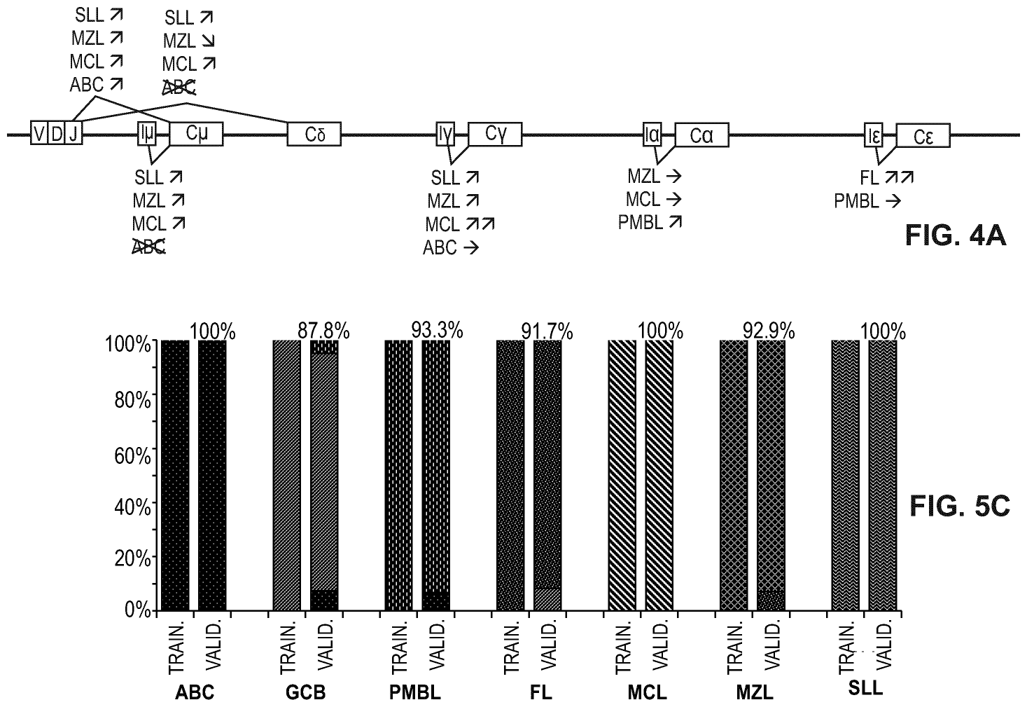

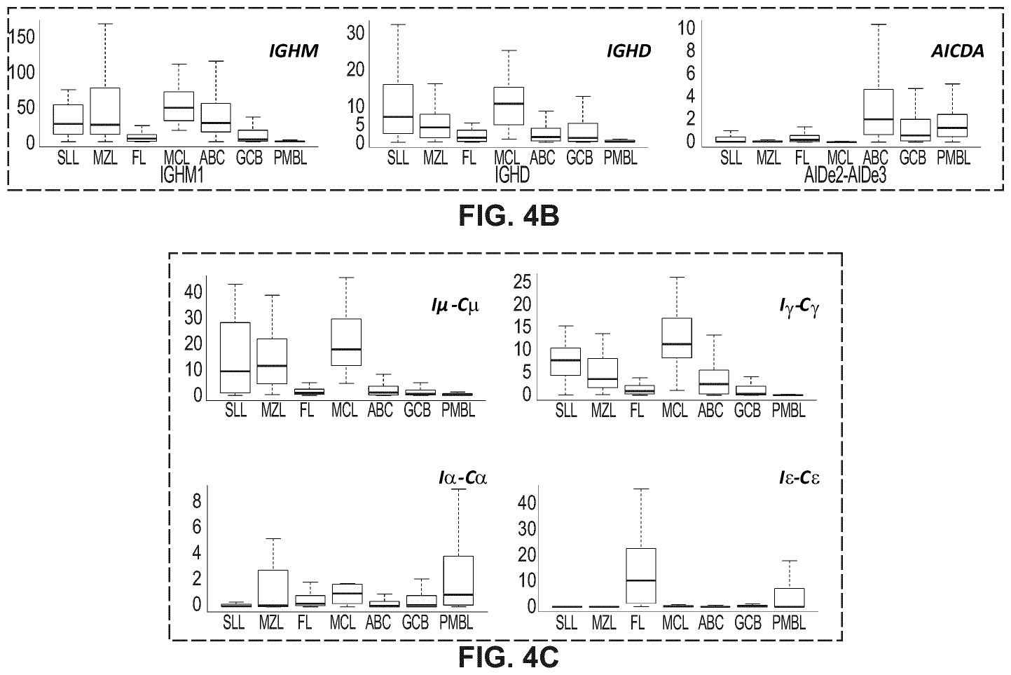

In addition to their cellular origin and the composition of their microenvironment, B-cell NHLs also differ in the configurations of their immunoglobulin genes. As shown in FIGS. 4A-4C, MCL and SLL can be distinguished from other B-NHLs based on the expression of the IGHD gene. Two groups of tumors can also be defined according to the expression of the IGHM gene. The first corresponds to the IGHM-positive tumors with an activated or memory B-cell origin (most ABC DLBCLs, MCL, MZL and SLL). The second corresponds to the tumors of GCB origin (particularly, GCB DLBCLs and FL), which often undergo isotype switching, and PMBLs, which usually lack immunoglobulin expression. Interestingly, the data also confirmed the existence of a class switch recombination (CSR) defect in ABC DLBCLs. As previously reported, the data confirmed that a majority of these tumors paradoxically express the IGHM gene along with AICDA, a direct activator of immunoglobulin isotype switching. The inventors evaluated the expression of the immunoglobulin sterile transcripts required for CSR activation to clarify this issue and observed that the expression of AICDA and the Iμ-Cμ transcript, which controls the accessibility of the switch μ region to the CSR machinery, are specifically desynchronized in these tumors. This Iμ-Cμ transcript is expressed by a majority of IgM-positive NHLs (SLLs, MZLs and MCLs), which do not express AICDA, but is downregulated in ABC DLBCLs, preventing isotype switching despite of AICDA expression. Surprisingly, the inventors also observed that the Iγ-Cγ sterile transcript is expressed at a high level in SLL and MCL, two nongerminal center-derived lymphomas, and the Iε-Cε transcript is almost exclusively expressed in FLs, constituting one of the most discriminatory markers for this pathology in the assay.

Development of a Random Forest Pan-B NHL Classifier

The inventors next trained a random forest (RF) classifier to discriminate the seven principal subtypes of B-cell NHLs in order to translate the results obtained above into a clinically applicable assay. DLBCLs with an ambiguous classification (inconclusive cell-of-origin classification by RT-MLPA and/or Nanostring Lymph2Cx), EBV-positive DLBCLs, and grade 3B FLs were excluded from the training. The 429 remaining cases were randomly assigned to a training cohort of 283 cases (two-thirds) and to a validation cohort of 146 cases (one-third). The training cohort comprised 190 DLBCLs (76 ABC, 86 GCB and 28 PMBL cases) that were previously classified by IHC and/or RT-MLPA, 35 FLs (grade 1 to 3A), 21 MCLs, 12 SLLs, and 25 cases in the MZL category (13 MZLs, 8 MALT lymphomas and 4 LPLs). The validation series comprised the 90 DLBCLs from the SENIOR trial classified as GCB (41 cases) or ABC (49 cases) DLBCLs by the Nanostring Lymph2Cx assay, 15 PMBLs, 12 grade 1 to 3A FLs, 10 MCLs, 5 SLLs and 14 MZLs (7 MZL, 3 MALT and 4 LPL).

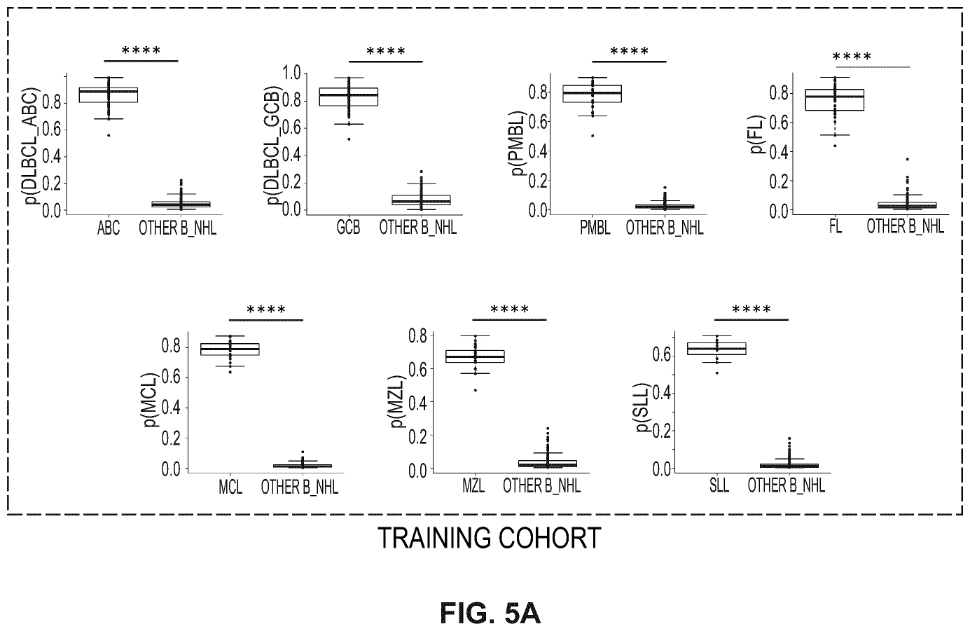

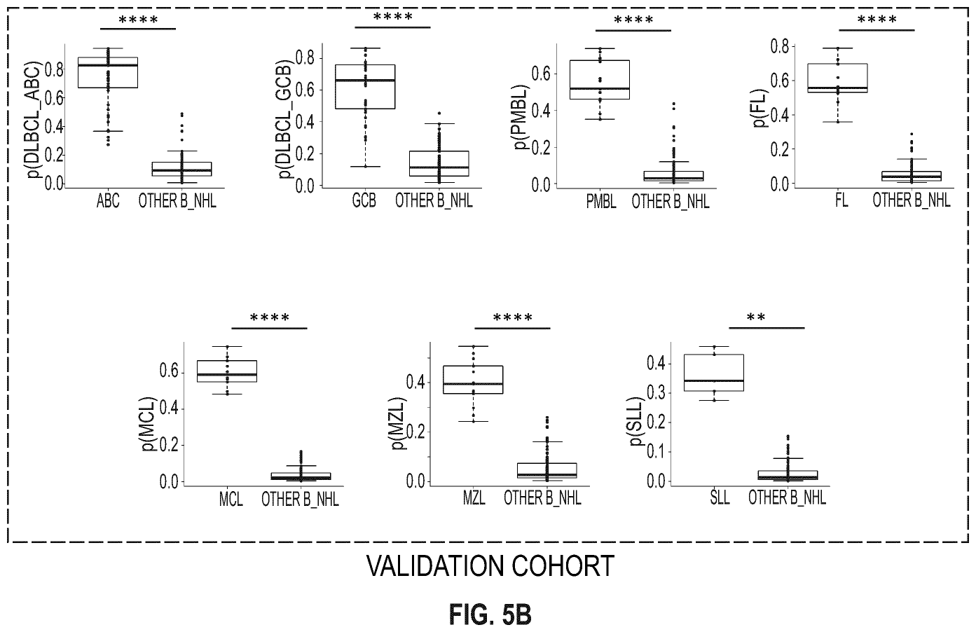

The RF algorithm classified all 283 cases of the training series into the expected subtype. As shown in FIG. 5A, the distributions of the probabilities that the tumor belonged to one of the seven subclasses indicated a very good capacity of the algorithm to discriminate these lymphomas. The RF predictor also classified 138/146 (94.5%) of the samples in the independent validation cohort into the expected subtype, showing a very good generalization capacity (FIG. 5B). For the ABC and GCB DLBCLs, the concordance with the Lymph2Cx assay in the validation cohort was 94.3%. The method agreed with the Lymph2Cx assay for 49/49 (100%) ABC DLBCLs and 36/41 (87.8%) GCB DLBCLs. Two cases classified as GCB DLBCLs by the Lymph2Cx assay were classified as PMBL by the RF predictor. Further analyses of these two cases identified genomic mutations compatible with the PMBL diagnosis, which is not addressed by the Lymph2Cx assay (B2M, TNFRSF14, SOX11 and CIITA mutations for one case; STATE, B2M, CD58, CIITA and CARD11 mutations for the other). The three other discordant cases were classified as ABC by the RF predictor, but no COO-specific mutations were detected in these samples. Notably, 14/15 PMBLs (93.3%) and 39/41 (95.1%) small cell lymphomas in the validation cohort were accurately classified, including all MCLs and SLLs. One FL was classified as a GCB DLBCL and one MZL as a FL, due to its preeminent GCB signature. Interestingly, 5 of the 8 FL3B tumors, which the inventors had excluded from the model building, were classified as DLBCLs by the RF predictor (3 GCB and 2 ABC cases), while 3 were classified as FLs. Otherwise, 5 of the 6 DLBCLs defined as unclassified by the Lymph2Cx assay were classified as ABC DLBCLs, including two samples harboring a CD79B mutation, which is usually associated with the ABC signature, and the last case was classified as GCB DLBCL, without COO-specific mutations detected (ARID1A and CDKN2A).

DLBCL Survival Analyses

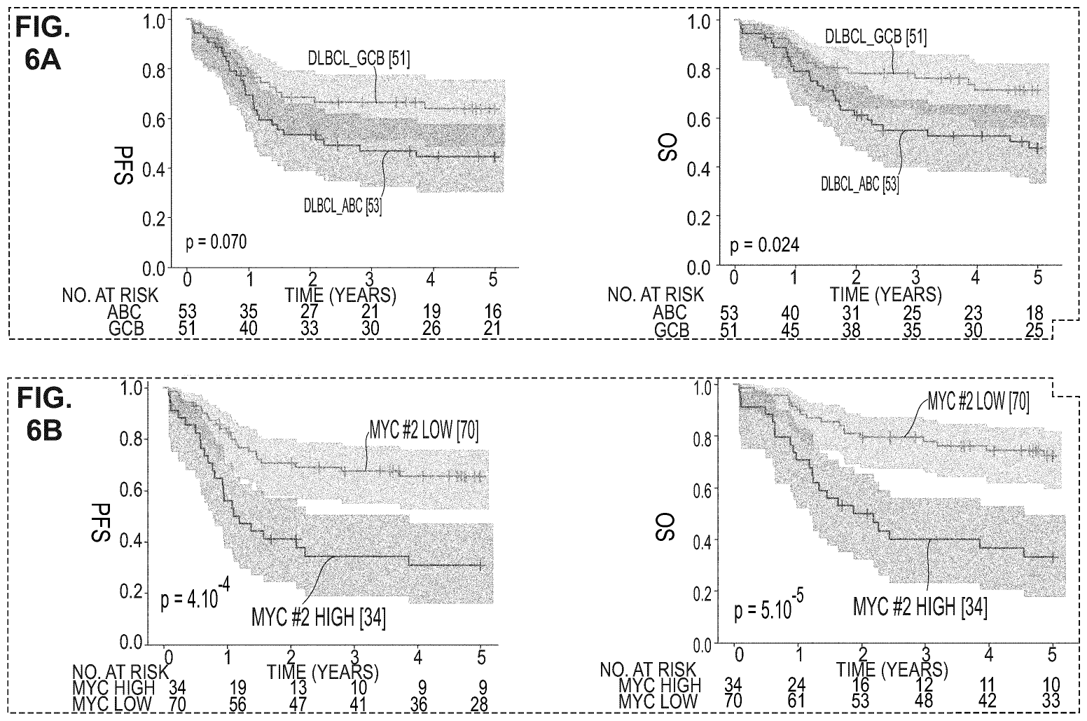

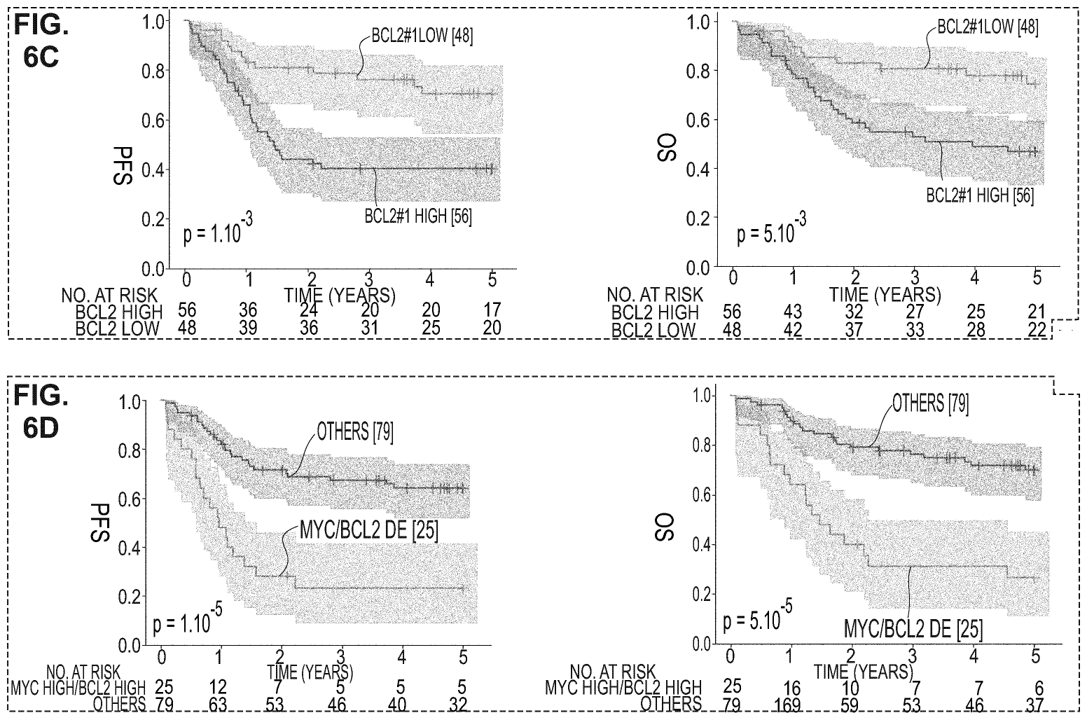

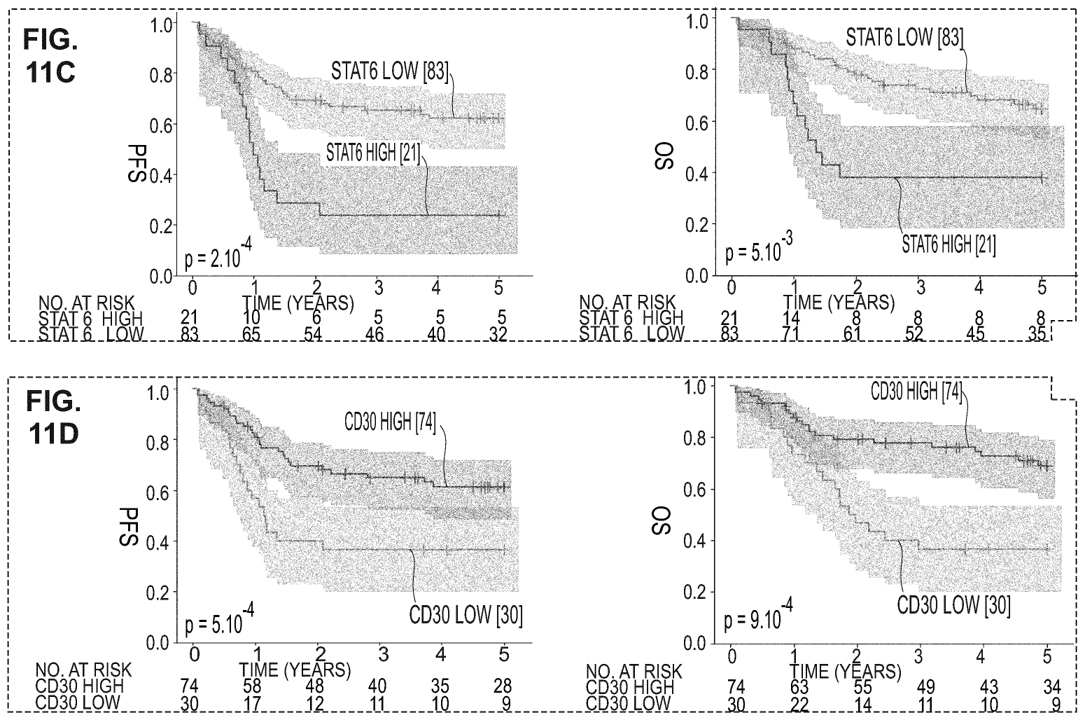

The inventors next focused on the 104 patients with DLBCL who were treated with a combination of rituximab and chemotherapy at the Centre Henri Becquerel to further evaluate the clinical value of the assay. In this cohort, the ABC/GCB COO was associated with OS (p=0.0306), but only a trend was observed with PFS (p=0.0899) (FIG. 6A). As shown in FIGS. 6B-6C, MYC and BCL2 expression were both associated with poorer PFS and OS, and the combination of the two identified a group of double-positive cases (24% of patients) with a particularly poor outcome (PFS, p<10⁻⁴ and OS, p<10⁻⁴) (FIG. 6D). This observation was confirmed with a multivariable model adjusted for the IPI score and cell-of-origin classification for both OS (HR, 2.08, 95% CI, 1.34 to 3.25, p<5·10⁻³) and PFS (HR, 2.04, 95% CI, 1.35 to 3.12, p<5-10⁻³), independent of the IPI (OS HR, 2.20, 95% CI, 1.41 to 3.41, p<5·10⁻³; PFS HR, 1.92, 95% CI, 1.27 to 2.89, p<5·10⁻³ (Table I). Clinical and biological characteristics of these patients, presented in Table II, identified significant correlations between the MYC/BCL2 double positive status and higher age (p=5·10⁻³), elevated LDH levels (p=0.04) and ABC subtype (p<10⁻⁴), in accordance with previous studies. (See Staiger A M, Ziepert M, Horn H, Scott D W, Barth T F E, Bernd H-W, et al. Clinical Impact of the Cell-of-Origin Classification and the MYC/BCL2 Dual Expresser Status in Diffuse Large B-Cell Lymphoma Treated Within Prospective Clinical Trials of the German High-Grade Non-Hodgkin's Lymphoma Study Group. J Clin Oncol. 1 août 2017; 35(22):2515-26; and Green T M, Young K H, Visco C, Xu-Monette Z Y, Orazi A, Go R S, et al. Immunohistochemical double-hit score is a strong predictor of outcome in patients with diffuse large B-cell lymphoma treated with rituximab plus cyclophosphamide, doxorubicin, vincristine, and prednisone. J Clin Oncol. 1 Oct. 2012; 30(28):3460-7.) As shown in FIG. 11, the expression of other RNA markers was also strongly correlated with PFS and OS in this cohort, including CARD11 (PFS, p<10⁻³ and OS, p<10⁻⁴), CREB3L2 (PFS, p<10⁻⁴ and OS, p<10⁻⁴), CD30 (PFS, p<10⁻² and OS, p<10⁻³) and STATE (PFS, p<10⁻³ and OS, p<10⁻²).

Tables XVI and XVII together identify:
HGCN—the official name of the marker (HUGO Gene Nomenclature Committee);
The Ensembl Accession number;
CCDSS or RefSeq (for NCBI database to find the sequence);
Aliases of each gene; and
The probe and gene specific elements of the specific sequence that was identified.

All references in the tables to public databases incorporate by reference the referenced sequences from those databases in their entireties.

TABLE XVI

| HGCN | Description | Ensembl Accession | CCDCS/RefSeq | Alias |
|---|---|---|---|---|
| AICDA | activation induced cytidine deaminase | ENSG00000111732 | CCDS41747 | AID |
| AICDA | activation induced cytidine deaminase | ENSG00000111732 | CCDS41747 | AID |
| AICDA | activation induced cytidine deaminase | ENSG00000111732 | CCDS41747 | AID |
| AICDA | activation induced cytidine deaminase | ENSG00000111732 | CCDS41747 | AID |
| ALK | ALK receptor tyrosine kinase | ENSG00000171094 | CCDS33172 | ALK |
| ALK | ALK receptor tyrosine kinase | ENSG00000171094 | CCDS33172 | ALK |
| ANXA1 | annexin A1 | ENSG00000135046 | CCDS6645 | ANXA1 |
| ANXA1 | annexin A1 | ENSG00000135046 | CCDS6645 | ANXA1 |
| ASB13 | ankyrin repeat and SOCS box | ENSG00000196372 | CCDS7070 | ASB13 |
| | containing 13 | | | |
| ASB13 | ankyrin repeat and SOCS box | ENSG00000196372 | CCDS7070 | ASB13 |
| | containing 13 | | | |
| B2M | beta-2-microglobulin | ENSG00000166710 | CCDS10113 | B2M |
| B2M | beta-2-microglobulin | ENSG00000166710 | CCDS10113 | B2M |
| BANK1 | B cell scaffold protein with ankyrin | ENSG00000153064 | CCDS34038 | BANK |
| | repeats 1 | | | |
| BANK1 | B cell scaffold protein with ankyrin | ENSG00000153064 | CCDS34038 | BANK |
| | repeats 1 | | | |
| BCL2 | BCL2 apoptosis regulator | ENSG00000171791 | CCDS11981 | BCL2 |
| BCL2 | BCL2 apoptosis regulator | ENSG00000171791 | CCDS11981 | BCL2 |
| BCL2 | BCL2 apoptosis regulator | ENSG00000171791 | CCDS11981 | BCL2 |
| BCL2 | BCL2 apoptosis regulator | ENSG00000171791 | CCDS11981 | BCL2 |
| BCL6 | BCL6 transcription repressor | ENSG00000113916 | CCDS3289 | BCL6 |
| BCL6 | BCL6 transcription repressor | ENSG00000113916 | CCDS3289 | BCL6 |
| BCL6 | BCL6 transcription repressor | ENSG00000113916 | CCDS3289 | BCL6 |
| BCL6 | BCL6 transcription repressor | ENSG00000113916 | CCDS3289 | BCL6 |
| BRAF | B-Raf proto-oncogene, serine/threonine | ENSG00000157764 | CCDS5863 | BRAFV600E |
| | kinase | | | |
| BRAF | B-Raf proto-oncogene, serine/threonine | ENSG00000157764 | CCDS5863 | BRAFV600E |
| | kinase | | | |
| CARD11 | caspase recruitment domain family | ENSG00000198286 | CCDS5336 | CARD11 |
| | member 11 | | | |
| CARD11 | caspase recruitment domain family | ENSG00000198286 | CCDS5336 | CARD11 |
| | member 11 | | | |
| CCDC50 | coiled-coil domain containing 50 | ENSG00000152492 | CCDS33912 | CCDC50 |
| CCDC50 | coiled-coil domain containing 50 | ENSG00000152492 | CCDS33912 | CCDC50 |
| CCND1 | cyclin D1 | ENSG00000110092 | CCDS8191 | CCND1 |
| CCND1 | cyclin D1 | ENSG00000110092 | CCDS8191 | CCND1 |
| CCND2 | cyclin D2 | ENSG00000118971 | CCDS8524 | CCND2 |
| CCND2 | cyclin D2 | ENSG00000118971 | CCDS8524 | CCND2 |
| CCR4 | C-C motif chemokine receptor 4 | ENSG00000183813 | CCDS2656 | CCR4 |
| CCR4 | C-C motif chemokine receptor 4 | ENSG00000183813 | CCDS2656 | CCR4 |
| CCR7 | C-C motif chemokine receptor 7 | ENSG00000126353 | CCDS11369 | CCR7 |
| CCR7 | C-C motif chemokine receptor 7 | ENSG00000126353 | CCDS11369 | CCR7 |
| CD163 | CD163 molecule | ENSG00000177575 | CCDS8578 | CD163 |
| CD163 | CD163 molecule | ENSG00000177575 | CCDS8578 | CD163 |
| CD19 | CD19 molecule | ENSG00000177455 | CCDS10644 | CD19 |
| CD19 | CD19 molecule | ENSG00000177455 | CCDS10644 | CD19 |
| CD22 | CD22 molecule | ENSG00000012124 | CCDS12457 | CD22 |
| CD22 | CD22 molecule | ENSG00000012124 | CCDS12457 | CD22 |
| CD27 | CD27 molecule | ENSG00000139193 | CCDS8545 | CD27 |
| CD27 | CD27 molecule | ENSG00000139193 | CCDS8545 | CD27 |
| CD274 | CD274 molecule | ENSG00000120217 | CCDS6464 | PDL1 |
| CD274 | CD274 molecule | ENSG00000120217 | CCDS6464 | PDL1 |
| CD28 | CD28 molecule | ENSG00000178562 | CCDS2361 | CD28 |
| CD28 | CD28 molecule | ENSG00000178562 | CCDS2361 | CD28 |
| CD3E | CD3e molecule | ENSG00000198851 | CCDS31685 | CD3 |
| CD3E | CD3e molecule | ENSG00000198851 | CCDS31685 | CD3 |
| CD4 | CD4 molecule | ENSG00000010610 | CCDS8562 | CD4 |
| CD4 | CD4 molecule | ENSG00000010610 | CCDS8562 | CD4 |
| CD40 | CD40 molecule | ENSG00000101017 | CCDS13393 | CD40 |
| CD40 | CD40 molecule | ENSG00000101017 | CCDS13393 | CD40 |
| CD40LG | CD40 ligand | ENSG00000102245 | CCDS14659 | CD40L |
| CD40LG | CD40 ligand | ENSG00000102245 | CCDS14659 | CD40L |
| CD40LG | CD40 ligand | ENSG00000102245 | CCDS14659 | CD40L |
| CD40LG | CD40 ligand | ENSG00000102245 | CCDS14659 | CD40L |
| CD5 | CD5 molecule | ENSG00000110448 | CCDS8000 | CD5 |
| CD5 | CD5 molecule | ENSG00000110448 | CCDS8000 | CD5 |
| CD68 | CD68 molecule | ENSG00000129226 | CCDS11114 | CD68 |
| CD68 | CD68 molecule | ENSG00000129226 | CCDS11114 | CD68 |
| CD70 | CD70 molecule | ENSG00000125726 | CCDS12170 | CD70 |
| CD70 | CD70 molecule | ENSG00000125726 | CCDS12170 | CD70 |
| CD80 | CD80 molecule | ENSG00000121594 | CCDS2989 | CD80 |
| CD80 | CD80 molecule | ENSG00000121594 | CCDS2989 | CD80 |
| CD86 | CD86 molecule | ENSG00000114013 | CCDS3009 | CD38 |
| CD86 | CD86 molecule | ENSG00000114013 | CCDS3009 | CD86 |
| CD86 | CD86 molecule | ENSG00000114013 | CCDS3009 | CD38 |
| CD86 | CD86 molecule | ENSG00000114013 | CCDS3009 | CD86 |
| CD8A | CD8a molecule | ENSG00000153563 | CCDS1992 | CD8 |
| CD8A | CD8a molecule | ENSG00000153563 | CCDS1992 | CD8 |
| CRBN | cereblon | ENSG00000113851 | CCDS2562 | CRBN |
| CRBN | cereblon | ENSG00000113851 | CCDS2562 | CRBN |
| CREB3L2 | cAMP responsive element binding | ENSG00000182158 | CCDS34760 | CREB3L2 |
| | protein 3 like 2 | | | |
| CREB3L2 | cAMP responsive element binding | ENSG00000182158 | CCDS34760 | CREB3L2 |
| | protein 3 like 2 | | | |
| CTLA4 | cytotoxic T-lymphocyte associated | ENSG00000163599 | CCDS2362 | CTLA4 |
| | protein 4 | | | |
| CTLA4 | cytotoxic T-lymphocyte associated | ENSG00000163599 | CCDS2362 | CTLA4 |
| | protein 4 | | | |
| CXCL13 | C—X—C motif chemokine ligand 13 | ENSG00000156234 | CCDS3582 | CXCL13 |
| CXCL13 | C—X—C motif chemokine ligand 13 | ENSG00000156234 | CCDS3582 | CXCL13 |
| CXCR5 | C—X—C motif chemokine receptor 5 | ENSG00000160683 | CCDS8402 | CXCR5 |
| CXCR5 | C—X—C motif chemokine receptor 5 | ENSG00000160683 | CCDS8402 | CXCR5 |
| CYB5R2 | cytochrome b5 reductase 2 | ENSG00000166394 | CCDS7780 | CYB5R2 |
| CYB5R2 | cytochrome b5 reductase 2 | ENSG00000166394 | CCDS7780 | CYB5R2 |
| DUSP22 | dual specificity phosphatase 22 | ENSG00000112679 | CCDS4468 | DUSP22 |
| DUSP22 | dual specificity phosphatase 22 | ENSG00000112679 | CCDS4468 | DUSP22 |
| EBER1 | Epstein-Barr virus-encoded small | n.a (Viral Genome) | GenBank: | EBER1 |
| | RNAs 1 | | AF200364.1 | |
| EBER1 | Epstein-Barr virus-encoded small | n.a (Viral Genome) | GenBank: | EBER1 |
| | RNAs 1 | | AF200364.1 | |
| FAS | Fas cell surface death receptor | ENSG00000026103 | CCDS7393 | CD95 |
| FAS | Fas cell surface death receptor | ENSG00000026103 | CCDS7393 | CD95 |
| FCER2 | Fc fragment of IgE receptor II | ENSG00000104921 | CCDS12184 | CD23 |
| FCER2 | Fc fragment of IgE receptor II | ENSG00000104921 | CCDS12184 | CD23 |
| FGFR1 | fibroblast growth factor receptor 1 | ENSG00000077782 | CCDS6107 | FGFR1 |
| FGFR1 | fibroblast growth factor receptor 1 | ENSG00000077782 | CCDS6107 | FGFR1 |
| FOXP1 | forkhead box P1 | ENSG00000114861 | CCDS2914 | FOXP1 |
| FOXP1 | forkhead box P1 | ENSG00000114861 | CCDS2914 | FOXP1 |
| FOXP3 | forkhead box P3 | ENSG00000049768 | CCDS14323 | FOXP3 |
| FOXP3 | forkhead box P3 | ENSG00000049768 | CCDS14323 | FOXP3 |
| GATA3 | GATA binding protein 3 | ENSG00000107485 | CCDS7083 | GATA3 |
| GATA3 | GATA binding protein 3 | ENSG00000107485 | CCDS7083 | GATA3 |
| GZMB | granzyme B | ENSG00000100453 | CCDS9633 | GRB |
| GZMB | granzyme B | ENSG00000100453 | CCDS9633 | GRB |
| HBZ | HTLV-1 basic zipper factor | n.a (Viral Genome) | GenBank: | HTLV1 |
| | | | KF053885.1 | |
| HBZ | HTLV-1 basic zipper factor | n.a (Viral Genome) | GenBank: | HTLV1 |
| | | | KF053885.1 | |
| ICOS | inducible T cell costimulator | ENSG00000163600 | CCDS2363 | ICOS |
| ICOS | inducible T cell costimulator | ENSG00000163600 | CCDS2363 | ICOS |
| IDH2 | isocitrate dehydrogenase (NADP(+)) 2, | ENSG00000182054 | CCDS10359 | IDH2R172K |
| | mitochondrial | | | |
| IDH2 | isocitrate dehydrogenase (NADP(+)) 2, | ENSG00000182054 | CCDS10359 | IDH2R172T |
| | mitochondrial | | | |
| IDH2 | isocitrate dehydrogenase (NADP(+)) 2, | ENSG00000182054 | CCDS10359 | IDH2R172 |
| | mitochondrial | | | |
| IFNG | interferon gamma | ENSG00000111537 | CCDS8980 | INFg |
| IFNG | interferon gamma | ENSG00000111537 | CCDS8980 | INFg |
| IGH | immunoglobulin heavy locus | n.a. (immunoglobulin) | NG_001019 | JH |
| IGH | immunoglobulin heavy locus | n.a. (immunoglobulin) | NG_001019 | Imu |
| IGH | immunoglobulin heavy locus | n.a. (immunoglobulin) | NG_001019 | Igamma |
| IGH | immunoglobulin heavy locus | n.a. (immunoglobulin) | NG_001019 | Ialpha |
| IGH | immunoglobulin heavy locus | n.a. (immunoglobulin) | NG_001019 | Iepsilon |
| IGH | immunoglobulin heavy locus | ENSG00000211899 | NG_001019 | Cmu |
| IGH | immunoglobulin heavy locus | ENSG00000211897 | NG_001019 | Cgamma |
| IGH | immunoglobulin heavy locus | ENSG00000211890 | NG_001019 | Calpha |
| IGH | immunoglobulin heavy locus | ENSG00000211891 | NG_001019 | Cepsilon |
| IGHD | immunoglobulin heavy constant delta | ENSG00000211898 | NG_001019 | IGHD |
| IGHD | immunoglobulin heavy constant delta | ENSG00000211898 | NG_001019 | IGHD |
| IGHM | immunoglobulin heavy constant mu | ENSG00000211899 | NG_001019 | IGHM |
| IGHM | immunoglobulin heavy constant mu | ENSG00000211899 | NG_001019 | IGHM |
| IL4I1 | interleukin 4 induced 1 | ENSG00000104951 | CCDS12786 | IL4I1 |
| IL4I1 | interleukin 4 induced 1 | ENSG00000104951 | CCDS12786 | IL4I1 |
| IRF4 | interferon regulatory factor 4 | ENSG00000137265 | CCDS4469 | IRF4 |
| IRF4 | interferon regulatory factor 4 | ENSG00000137265 | CCDS4469 | IRF4 |
| ITPKB | inositol-trisphosphate 3-kinase B | ENSG00000143772 | CCDS1555 | ITPKB |
| ITPKB | inositol-trisphosphate 3-kinase B | ENSG00000143772 | CCDS1555 | ITPKB |
| JAK2 | Janus kinase 2 | ENSG00000096968 | CCDS6457 | JAK2 |
| JAK2 | Janus kinase 2 | ENSG00000096968 | CCDS6457 | JAK2 |
| LAG3 | lymphocyte activating 3 | ENSG00000089692 | CCDS8561 | LAG3 |
| LAG3 | lymphocyte activating 3 | ENSG00000089692 | CCDS8561 | LAG3 |
| LIMD1 | LIM domains containing 1 | ENSG00000144791 | CCDS2729 | LIMD1 |
| LIMD1 | LIM domains containing 1 | ENSG00000144791 | CCDS2729 | LIMD1 |
| LMO2 | LIM domain only 2 | ENSG00000135363 | CCDS7888 | LMO2 |
| LMO2 | LIM domain only 2 | ENSG00000135363 | CCDS7888 | LMO2 |
| MAL | mal, T cell differentiation protein | ENSG00000172005 | CCDS2006 | MAL |
| MAL | mal, T cell differentiation protein | ENSG00000172005 | CCDS2006 | MAL |
| MAML3 | mastermind like transcriptional | ENSG00000196782 | CCDS54805 | MAML3 |
| | coactivator 3 | | | |
| MAML3 | mastermind like transcriptional | ENSG00000196782 | CCDS54805 | MAML3 |
| | coactivator 3 | | | |
| MEF2B | myocyte enhancer factor 2B | ENSG00000213999 | CCDS12394 | MEF2B |
| MEF2B | myocyte enhancer factor 2B | ENSG00000213999 | CCDS12394 | MEF2B |
| MKI67 | marker of proliferation Ki-67 | ENSG00000148773 | CCDS7659 | KI67 |
| MKI67 | marker of proliferation Ki-67 | ENSG00000148773 | CCDS7659 | KI67 |
| MME | membrane metalloendopeptidase | ENSG00000196549 | CCDS3172 | CD10 |
| MME | membrane metalloendopeptidase | ENSG00000196549 | CCDS3172 | CD10 |
| MS4A1 | membrane spanning 4-domains A1 | ENSG00000156738 | CCDS31570 | MS4A1 |
| MS4A1 | membrane spanning 4-domains A1 | ENSG00000156738 | CCDS31570 | MS4A1 |
| MYBL1 | MYB proto-oncogene like 1 | ENSG00000185697 | CCDS47867 | MYBL1 |
| MYBL1 | MYB proto-oncogene like 1 | ENSG00000185697 | CCDS47867 | MYBL1 |
| MYC | MYC proto-oncogene, bHLH | ENSG00000136997 | CCDS6359 | MYC |
| | transcription factor | | | |
| MYC | MYC proto-oncogene, bHLH | ENSG00000136997 | CCDS6359 | MYC |
| | transcription factor | | | |
| MYC | MYC proto-oncogene, bHLH | ENSG00000136997 | CCDS6359 | MYC |
| | transcription factor | | | |
| MYC | MYC proto-oncogene, bHLH | ENSG00000136997 | CCDS6359 | MYC |
| | transcription factor | | | |
| MYD88 | MYD88 innate immune signal | ENSG00000172936 | CCDS2674 | MYD88 |
| | transduction adaptor | | | |
| MYD88 | MYD88 innate immune signal | ENSG00000172936 | CCDS2674 | MYD88 |
| | transduction adaptor | | | |
| MYD88 | MYD88 innate immune signal | ENSG00000172936 | CCDS2674 | MYD88 |
| | transduction adaptor | | | |
| MYD88 | MYD88 innate immune signal | ENSG00000172936 | CCDS2674 | MYD88 |
| | transduction adaptor | | | |
| NCAM1 | neural cell adhesion molecule 1 | ENSG00000149294 | CCDS73384 | CD56 |
| NCAM1 | neural cell adhesion molecule 1 | ENSG00000149294 | CCDS73384 | CD56 |
| NEK6 | NIMA related kinase 6 | ENSG00000119408 | CCDS6854 | NEK6 |
| NEK6 | NIMA related kinase 6 | ENSG00000119408 | CCDS6854 | NEK6 |
| PDCD1 | programmed cell death 1 | ENSG00000188389 | CCDS33428 | PD1 |
| PDCD1 | programmed cell death 1 | ENSG00000188389 | CCDS33428 | PD1 |
| PDCD1LG2 | programmed cell death 1 ligand 2 | ENSG00000197646 | CCDS6465 | PDL2 |
| PDCD1LG2 | programmed cell death 1 ligand 2 | ENSG00000197646 | CCDS6465 | PDL2 |
| PIM2 | Pim-2 proto-oncogene, serine/threonine | ENSG00000102096 | CCDS14312 | P1M2 |
| | kinase | | | |
| PIM2 | Pim-2 proto-oncogene, serine/threonine | ENSG00000102096 | CCDS14312 | P1M2 |
| | kinase | | | |
| PRDM1 | PR/SET domain 1 | ENSG00000057657 | CCDS5054 | PRDM1 |
| PRDM1 | PR/SET domain 1 | ENSG00000057657 | CCDS5054 | PRDM1 |
| PRF1 | perforin 1 | ENSG00000180644 | CCDS7305 | PRF |
| PRF1 | perforin 1 | ENSG00000180644 | CCDS7305 | PRF |
| PTPRC | protein tyrosine phosphatase receptor | ENSG00000081237 | CCD51397 | CD45R0 |
| | type C | | | |
| PTPRC | protein tyrosine phosphatase receptor | ENSG00000081237 | CCD51397 | CD45R0 |
| | type C | | | |
| RAB29 | RAB29, member RAS oncogene family | ENSG00000117280 | CCDS1459 | RAB7L1 |
| RAB29 | RAB29, member RAS oncogene family | ENSG00000117280 | CCDS1459 | RAB7L1 |
| RHOA | ras homolog family member A | ENSG00000067560 | CCDS2795 | RHOAG17V |
| RHOA | ras homolog family member A | ENSG00000067560 | CCDS2795 | RHOAG17V |
| S1PR2 | sphingosine-1-phosphate receptor 2 | ENSG00000267534 | CCDS12229 | S1PR2 |
| S1PR2 | sphingosine-1-phosphate receptor 2 | ENSG00000267534 | CCDS12229 | S1PR2 |
| SDC1 | syndecan 1 | ENSG00000115884 | CCDS1697 | CD138 |
| SDC1 | syndecan 1 | ENSG00000115884 | CCDS1697 | CD138 |
| SERPINA9 | serpin family A member 9 | ENSG00000170054 | CCDS41982 | SERPINA9 |
| SERPINA9 | serpin family A member 9 | ENSG00000170054 | CCDS41982 | SERPINA9 |
| SH3BP5 | SH3 domain binding protein 5 | ENSG00000131370 | CCD52625 | SH3BP5 |
| SH3BP5 | SH3 domain binding protein 5 | ENSG00000131370 | CCD52625 | SH3BP5 |
| STAT6 | signal transducer and activator of | ENSG00000166888 | CCDS8931 | STAT6 |
| | transcription 6 | | | |
| STAT6 | signal transducer and activator of | ENSG00000166888 | CCDS8931 | STAT6 |
| | transcription 6 | | | |
| TBX21 | T-box transcription factor 21 | ENSG00000073861 | CCDS11514 | TBET |
| TBX21 | T-box transcription factor 21 | ENSG00000073861 | CCDS11514 | TBET |
| TCL1A | T cell leukemia/lymphoma 1A | ENSG00000100721 | CCDS9941 | TCL1A |
| TCL1A | T cell leukemia/lymphoma 1A | ENSG00000100721 | CCDS9941 | TCL1A |
| TFRC | transferrin receptor | ENSG00000072274 | CCDS3312 | CD71 |
| TFRC | transferrin receptor | ENSG00000072274 | CCDS3312 | CD71 |
| TNFRSF13B | TNF receptor superfamily member 13B | ENSG00000240505 | CCDS11181 | TACI |
| TNFRSF13B | TNF receptor superfamily member 13B | ENSG00000240505 | CCDS11181 | TACI |
| TNFRSF17 | TNF receptor superfamily member 17 | ENSG00000048462 | CCDS10552 | BCMA |
| TNFRSF17 | TNF receptor superfamily member 17 | ENSG00000048462 | CCDS10552 | BCMA |
| TNFRSF8 | TNF receptor superfamily member 8 | ENSG00000120949 | CCDS144 | CD30 |
| TNFRSF8 | TNF receptor superfamily member 8 | ENSG00000120949 | CCDS144 | CD30 |
| TNFSF13 | TNF superfamily member 13 | ENSG00000161955 | CCDS11111 | APRIL |
| TNFSF13 | TNF superfamily member 13 | ENSG00000161955 | CCDS11111 | APRIL |
| TNFSF13B | TNF superfamily member 13b | ENSG00000102524 | CCD59509 | BAFF |
| TNFSF13B | TNF superfamily member 13b | ENSG00000102524 | CCD59509 | BAFF |
| TRA | T cell receptor alpha locus | n.a. (immunoglobulin) | NG_001332 | TRAC |
| TRA | T cell receptor alpha locus | n.a. (immunoglobulin) | NG_001332 | TRAC |
| TRAF1 | TNF receptor associated factor 1 | ENSG00000056558 | CCD56825 | TRAF1 |
| TRAF1 | TNF receptor associated factor 1 | ENSG00000056558 | CCD56825 | TRAF1 |
| TRB | T cell receptor beta locus | n.a. (immunoglobulin) | NG_001333 | TCRbeta |
| TRB | T cell receptor beta locus | n.a. (immunoglobulin) | NG_001333 | TCRbeta |
| TRD | T cell receptor delta locus | n.a. (immunoglobulin) | NG_001332 | TCRdelta |
| TRD | T cell receptor delta locus | n.a. (immunoglobulin) | NG_001332 | TCRdelta |
| TRG | T cell receptor gamma locus | n.a. (immunoglobulin) | NG_001336 | TCRgamma |
| TRG | T cell receptor gamma locus | n.a. (immunoglobulin) | NG_001336 | TCRgamma |
| XBP1 | X-box binding protein 1 | ENSG00000100219 | CCDS13847 | XBP1 |
| XBP1 | X-box binding protein 1 | ENSG00000100219 | CCDS13847 | XBP1 |
| XPO1 | exportin 1 | ENSG00000082898 | CCD533205 | XPOE571K |
| XPO1 | exportin 1 | ENSG00000082898 | CCD533205 | XPOWT |
| XPO1 | exportin 1 | ENSG00000082898 | CCD533205 | XPOE571K |
| XPO1 | exportin 1 | ENSG00000082898 | CCD533205 | XPOWT |
| ZAP70 | zeta chain of T cell receptor associated | ENSG00000115085 | CCD533254 | ZAP70 |
| | protein kinase 70 | | | |
| ZAP70 | zeta chain of T cell receptor associated | ENSG00000115085 | CCD533254 | ZAP70 |
| | protein kinase 70 | | | |

TABLE XVII

| | | | Probe Sequence (gene | Seq |
|---|---|---|---|---|
| HGCN | Alias | Probe | specific: underline; adaptors: plain font) | ID NO: |
| AICDA | AID | 5′ | GTGCCAGCAAGATCCAATCTAGANNNNNNNTCACTGGACTTTGGTT | 1 |
| | | | ATCTTCGCAATAAG | |
| AICDA | AID | 5′ | GTGCCAGCAAGATCCAATCTAGANNNNNNNAGACAGCTTCGGCGC | 2 |
| | | | ATCCTTTTG | |
| AICDA | AID | 3′ | AACGGCTGCCACGTGGAATTGCTCCAACCCTTAGGGAACCC | 3 |
| AICDA | AID | 3′ | CCCCTGTATGAGGTTGATGACTTACGAGACGTCCAACCCTTAGGGA | 4 |
| | | | ACCC | |
| ALK | ALK | 5′ | GTGCCAGCAAGATCCAATCTAGANNNNNNNCCTCCGAGAGACCCG | 5 |
| | | | CCCTCGCCCG | |
| ALK | ALK | 3′ | AGCCAGCCCTCCTCCCTGGCCATGCTCCAACCCTTAGGGAACCC | 6 |
| ANXA1 | ANXA1 | 5′ | GTGCCAGCAAGATCCAATCTAGANNNNNNNCTGCCTTGCATAAGG | 7 |
| | | | CCATAATGGTTAAAG | |
| ANXA1 | ANXA1 | 3′ | GTGTGGATGAAGCAACCATCATTGACATTCTCCAACCCTTAGGGAA | 8 |
| | | | CCC | |
| ASB13 | ASB13 | 5′ | GTGCCAGCAAGATCCAATCTAGANNNNNNNGCACGAGGCCTGCAT | 9 |
| | | | GAGCG | |
| ASB13 | ASB13 | 3′ | GGAGTTCCGAATGTGTGAGGCTTCTTATTGTCCAACCCTTAGGGAA | 10 |
| | | | CCC | |
| B2M | B2M | 5′ | GTGCCAGCAAGATCCAATCTAGANNNNNNNCTTTGTCACAGCCCAA | 11 |
| | | | GATAGTTAAGTGGG | |
| B2M | B2M | 3′ | ATCGAGACATGTAAGCAGCATCATGGAGTCCAACCCTTAGGGAAC | 12 |
| | | | CC | |
| BANK1 | BANK | 5′ | GTGCCAGCAAGATCCAATCTAGANNNNNNNGAAAAAGTGGCCTGG | 13 |
| | | | AAATGATTCAGCAG | |
| BANK1 | BANK | 3′ | GAGAAATTACGACAACTACGAGACTGCATTTCCAACCCTTAGGGAA | 14 |
| | | | CCC | |
| BCL2 | BCL2 | 5′ | GTGCCAGCAAGATCCAATCTAGANNNNNNNCCTGGATCCAGGATA | 15 |
| | | | ACGGAGGCTGG | |
| BCL2 | BCL2 | 5′ | GTGCCAGCAAGATCCAATCTAGANNNNNNNAGAGGATCATGCTGT | 16 |
| | | | ACTTAAAAAATACAA | |
| BCL2 | BCL2 | 3′ | GATGCCTTTGTGGAACTGTACGGCCTCCAACCCTTAGGGAACCC | 17 |
| BCL2 | BCL2 | 3′ | CATCACAGAGGAAGTAGACTGATATTAACATCCAACCCTTAGGGAA | 18 |
| | | | CCC | |
| BCL6 | BCL6 | 5′ | GTGCCAGCAAGATCCAATCTAGANNNNNNNCATAAAACGGTCCTCA | 19 |
| | | | TGGCCTGCAG | |
| BCL6 | BCL6 | 5′ | GTGCCAGCAAGATCCAATCTAGANNNNNNNAAGAAGTTTCTAGGAA | 20 |
| | | | AGGCCGGACACCAG | |
| BCL6 | BCL6 | 3′ | TGGCCTGTTCTATAGCATCTTTACAGACCAGTTGTCCAACCCTTAG | 21 |
| | | | GGAACCC | |
| BCL6 | BCL6 | 3′ | GTTTTGAGCAAAATTTTGGACTGTGAAGCATCCAACCCTTAGGGAA | 22 |
| | | | CCC | |
| BRAF | BRAFV | 5′ | GTGCCAGCAAGATCCAATCTAGANNNNNNNAAAAATAGGTGATTTT | 23 |
| | 600E | | GGTCTAGCTACAGA | |
| BRAF | BRAFV | 3′ | GAAATCTCGATGGAGTGGGTCCCTCCAACCCTTAGGGAACCC | 24 |
| | 600E | | | |
| CARD1 | CARD1 | 5′ | GTGCCAGCAAGATCCAATCTAGANNNNNNNCCACTCGGAGATTCT | 25 |
| 1 | 1 | | CCACCATTGTGG | |
| CARD1 | CARD1 | 3′ | TGGAGGAAGGCCACGAGGGCCTCCAACCCTTAGGGAACCC | 26 |
| 1 | 1 | | | |
| CCDC5 | CCDC5 | 5′ | GTGCCAGCAAGATCCAATCTAGANNNNNNNGACGACGCATTCAGG | 27 |
| 0 | 0 | | AGAAGAAGGATGAG | |
| CCDC5 | CCDC5 | 3′ | GACATAGCTCGCCTTTTGCAAGAAAAGGAGTCCAACCCTTAGGGAA | 28 |
| 0 | 0 | | CCC | |
| CCND1 | CCND1 | 5′ | GTGCCAGCAAGATCCAATCTAGANNNNNNNACCTTCGTTGCCCTCT | 29 |
| | | | GTGCCACAG | |
| CCND1 | CCND1 | 3 | ATGTGAAGTTCATTTCCAATCCGCCCTTCCAACCCTTAGGGAACCC | 30 |
| CCND2 | CCND2 | 5′ | GTGCCAGCAAGATCCAATCTAGANNNNNNNGGTGGCCACCTGGAT | 31 |
| | | | GCTGGAG | |
| CCND2 | CCND2 | 3′ | GTCTGTGAGGAACAGAAGTGCGAAGAAGAGTCCAACCCTTAGGGA | 32 |
| | | | ACCC | |
| CCR4 | CCR4 | 5′ | GTGCCAGCAAGATCCAATCTAGANNNNNNNCCTCAGAGCCGCTTT | 33 |
| | | | CAGAAAAGCAAG | |
| CCR4 | CCR4 | 3′ | CTGCTTCTGGTTGGGCCCAGACCTTCCAACCCTTAGGGAACCC | 34 |
| CCR7 | CCR7 | 5′ | GTGCCAGCAAGATCCAATCTAGANNNNNNNGTGGTGGCTCTCCTT | 35 |
| | | | GTCATTTTCCAG | |
| CCR7 | CCR7 | 3′ | GTATGCCTGTGTCAAGATGAGGTCACGGTCCAACCCTTAGGGAAC | 36 |
| | | | CC | |
| CD163 | CD163 | 5′ | GTGCCAGCAAGATCCAATCTAGANNNNNNNAGAGCAAGTGGCCTC | 37 |
| | | | TGTAATCTGCTCAG | |
| CD163 | CD163 | 3′ | GAAACCAGTCCCAAACACTGTCCTCGTTCCAACCCTTAGGGAACCC | 38 |
| CD19 | CD19 | 5′ | GTGCCAGCAAGATCCAATCTAGANNNNNNNCTGGAGATCACTGCT | 39 |
| | | | CGGCCAG | |
| CD19 | CD19 | 3′ | TACTATGGCACTGGCTGCTGAGGACTGTCCAACCCTTAGGGAACC | 40 |
| | | | C | |
| CD22 | CD22 | 5′ | GTGCCAGCAAGATCCAATCTAGANNNNNNNGGATGGAACGAATAC | 41 |
| | | | ACCTCAATGTCTCTG | |
| CD22 | CD22 | 3′ | AAAGGCCTTTTCCACCTCATATCCAGCTCCTCCAACCCTTAGGGAA | 42 |
| | | | CCC | |
| CD27 | CD27 | 5′ | GTGCCAGCAAGATCCAATCTAGANNNNNNNCTCAGCCCACCCACT | 43 |
| | | | TACCTTATGTCAGTG | |
| CD27 | CD27 | 3′ | AGATGCTGGAGGCCAGGACAGCTGTCCAACCCTTAGGGAACCC | 44 |
| CD274 | PDL1 | 5′ | GTGCCAGCAAGATCCAATCTAGANNNNNNNAAAACCATACAGCTGA | 45 |
| | | | ATTGGTCATCCCAG | |
| CD274 | PDL1 | 3′ | AACTACCTCTGGCACATCCTCCAAATGAAATCCAACCCTTAGGGAA | 46 |
| | | | CCC | |
| CD28 | CD28 | 5′ | GTGCCAGCAAGATCCAATCTAGANNNNNNNTCAACTTATTCCCTTC | 47 |
| | | | AATTCAAGTAACAG | |
| CD28 | CD28 | 3′ | GAAACAAGATTTTGGTGAAGCAGTCGCCTCCAACCCTTAGGGAACC | 48 |
| | | | C | |
| CD3E | CD3 | 5′ | GTGCCAGCAAGATCCAATCTAGANNNNNNNTGCTGGCGGCAGGCA | 49 |
| | | | AAGGG | |
| CD3E | CD3 | 3′ | GACAAAACAAGGAGAGGCCACCACCTCCAACCCTTAGGGAACCC | 50 |
| CD4 | CD4 | 5′ | GTGCCAGCAAGATCCAATCTAGANNNNNNNGAGGAGGTGCAATTG | 51 |
| | | | CTAGTGTTCGGAT | |
| CD4 | CD4 | 3′ | TGACTGCCAACTCTGACACCCACCTTCCAACCCTTAGGGAACCC | 52 |
| CD40 | CD40 | 5′ | GTGCCAGCAAGATCCAATCTAGANNNNNNNCGGCTTTGGGGTCAA | 53 |
| | | | GCAGATTG | |
| CD40 | CD40 | 3′ | CTACAGGGGTTTCTGATACCATCTGCGAGTCCAACCCTTAGGGAAC | 54 |
| | | | CC | |
| CD40L | CD40L | 5′ | GTGCCAGCAAGATCCAATCTAGANNNNNNNAGAAAGAAAACAGCTT | 55 |
| G | | | TGAAATGCAAAAAG | |
| CD40L | CD40L | 5′ | GTGCCAGCAAGATCCAATCTAGANNNNNNNATTAAAAGCCAGTTTG | 56 |
| G | | | AAGGCTTTGTGAAG | |
| CD40L | CD40L | 3′ | TGTTACAGTGGGCTGAAAAAGGATACTACATCCAACCCTTAGGGAA | 57 |
| G | | | CCC | |
| CD40L | CD40L | 3′ | GATATAATGTTAAACAAAGAGGAGACGAAGTCCAACCCTTAGGGAA | 58 |
| G | | | CCC | |
| CD5 | CD5 | 5′ | GTGCCAGCAAGATCCAATCTAGANNNNNNNCCACCACAACTCCAG | 59 |
| | | | AGCCCACAG | |
| CD5 | CD5 | 3′ | CTCCTCCCAGGCTGCAGCTGGTCCAACCCTTAGGGAACCC | 60 |
| CD68 | CD68 | 5′ | GTGCCAGCAAGATCCAATCTAGANNNNNNNATGTACACAACCCAG | 61 |
| | | | GGTGGAGGAGAG | |
| CD68 | CD68 | 3′ | GCCTGGGGCATCTCTGTACTGAACCCTCCAACCCTTAGGGAACCC | 62 |
| CD70 | CD70 | 5′ | GTGCCAGCAAGATCCAATCTAGANNNNNNNGTAGCTGAGCTGCAG | 63 |
| | | | CTGAATCACACAG | |
| CD70 | CD70 | 3′ | GACCTCAGCAGGACCCCAGGCTATACTGTCCAACCCTTAGGGAAC | 64 |
| | | | CC | |
| CD80 | CD80 | 5′ | GTGCCAGCAAGATCCAATCTAGANNNNNNNGAAATTTATCATAACC | 65 |
| | | | GGTTTGATGCTGTG | |
| CD80 | CD80 | 3′ | CAATCTGCACATCGTGCTGCCACTCCAACCCTTAGGGAACCC | 66 |
| CD86 | CD38 | 5′ | GTGCCAGCAAGATCCAATCTAGANNNNNNNAGTATTCTGGAAAACG | 67 |
| | | | GTTTCCCGCAGG | |
| CD86 | CD86 | 5′ | GTGCCAGCAAGATCCAATCTAGANNNNNNNTCTTTGTGATGGCCTT | 68 |
| | | | CCTGCTCTCTG | |
| CD86 | CD38 | 3′ | TTTGCAGAAGCTGCCTGTGATGTGGTTCCAACCCTTAGGGAACCC | 69 |
| CD86 | CD86 | 3′ | GTGCTGCTCCTCTGAAGATTCAAGCTTATTTCCAACCCTTAGGGAA | 70 |
| | | | CCC | |
| CD8A | CD8 | 5′ | GTGCCAGCAAGATCCAATCTAGANNNNNNNTCGTGCCGGTCTTCC | 71 |
| | | | TGCCAG | |
| CD8A | CD8 | 3′ | CGAAGCCCACCACGACGCCTCCAACCCTTAGGGAACCC | 72 |
| CRBN | CRBN | 5′ | GTGCCAGCAAGATCCAATCTAGANNNNNNNGCCTTCTACAGAACA | 73 |
| | | | CAGCTGGTTTCCTGG | |
| CRBN | CRBN | 3′ | GTATGCCTGGACTGTTGCCCAGTGTAAGATTCCAACCCTTAGGGAA | 74 |
| | | | CCC | |
| CREB3 | CREB3 | 5′ | GTGCCAGCAAGATCCAATCTAGANNNNNNNGAGGAACCTCCTCTG | 75 |
| L2 | L2 | | GAAATGAACACTGGG | |
| CREB3 | CREB3 | 3′ | GTTGATTCCTCGTGCCAGACCATTATTCCTTCCAACCCTTAGGGAA | 76 |
| L2 | L2 | | CCC | |
| CTLA4 | CTLA4 | 5′ | GTGCCAGCAAGATCCAATCTAGANNNNNNNCTCCTCACAGCTGTTT | 77 |
| | | | CTTTGAGCAAAATG | |
| CTLA4 | CTLA4 | 3′ | CTAAAGAAAAGAAGCCCTCTTACAACAGGGTCCAACCCTTAGGGAA | 78 |
| | | | CCC | |
| CXCL1 | CXCL1 | 5′ | GTGCCAGCAAGATCCAATCTAGANNNNNNNGGTCAGCAGCCTCTC | 79 |
| 3 | 3 | | TCCAGTCCAAG | |
| CXCL1 | CXCL1 | 3′ | GTGTTCTGGAGGTCTATTACACAAGCTTGAGGTGTTCCAACCCTTA | 80 |
| 3 | 3 | | GGGAACCC | |
| CXCR5 | CXCR5 | 5′ | GTGCCAGCAAGATCCAATCTAGANNNNNNNGGACCTCGAGAACCT | 81 |
| | | | GGAGGACCTG | |
| CXCR5 | CXCR5 | 3′ | TTCTGGGAACTGGACAGATTGGACAACTATAACGTCCAACCCTTAG | 82 |
| | | | GGAACCC | |
| CYB5R | CYB5R | 5′ | GTGCCAGCAAGATCCAATCTAGANNNNNNNGGAATGATTGCTGGG | 83 |
| 2 | 2 | | GGCACAG | |
| CYB5R | CYB5R | 3′ | GCATCACACCCATGTTGCAGCTCATTCCAACCCTTAGGGAACCC | 84 |
| 2 | 2 | | | |
| DUSP2 | DUSP2 | 5′ | GTGCCAGCAAGATCCAATCTAGANNNNNNNCACGATAGTGCCAGG | 85 |
| 2 | 2 | | CCTATGTTGGAG | |
| DUSP2 | DUSP2 | 3′ | GGAGTTAAATACCTGTGCATCCCAGCAGCTCCAACCCTTAGGGAAC | 86 |
| 2 | 2 | | CC | |
| EBER1 | EBER1 | 5′ | GTGCCAGCAAGATCCAATCTAGANNNNNNNGTAGCCACCCGTCCC | 87 |
| | | | GGGTA | |
| EBER1 | EBER1 | 3′ | CAAGTCCCGGGTGGTGAGGATCCAACCCTTAGGGAACCC | 88 |
| FAS | CD95 | 5′ | GTGCCAGCAAGATCCAATCTAGANNNNNNNAATTCTGCCATAAGCC | 89 |
| | | | CTGTCCTCCAG | |
| FAS | CD95 | 3′ | GTGAAAGGAAAGCTAGGGACTGCACAGTCATCCAACCCTTAGGGA | 90 |
| | | | ACCC | |
| FCER2 | CD23 | 5′ | GTGCCAGCAAGATCCAATCTAGANNNNNNNGATGGAGTTGCAGGT | 91 |
| | | | GTCCAGCG | |
| FCER2 | CD23 | 3′ | GCTTTGTGTGCAACACGTGCCCTTCCAACCCTTAGGGAACCC | 92 |
| FGFR1 | FGFR1 | 5′ | GTGCCAGCAAGATCCAATCTAGANNNNNNNAACCACACATACCAG | 93 |
| | | | CTGGATGTCGTGG | |
| FGFR1 | FGFR1 | 3′ | AGCGGTCCCCTCACCGGCCCTCCAACCCTTAGGGAACCC | 94 |
| FOXP1 | FOXP1 | 5′ | GTGCCAGCAAGATCCAATCTAGANNNNNNNCCCTTCCCCTTCAACC | 95 |
| | | | TCTTGCTCAAG | |
| FOXP1 | FOXP1 | 3′ | GCATGATTCCAACAGAACTGCAGCAGCTCCAACCCTTAGGGAACC | 96 |
| | | | C | |
| FOXP3 | FOXP3 | 5′ | GTGCCAGCAAGATCCAATCTAGANNNNNNNGGACAGGCCACATTT | 97 |
| | | | CATGCACCAG | |
| FOXP3 | FOXP3 | 3′ | CTCTCAACGGTGGATGCCCACGCTCCAACCCTTAGGGAACCC | 98 |
| GATA3 | GATA3 | 5′ | GTGCCAGCAAGATCCAATCTAGANNNNNNNCCTCATTAAGCCCAA | 99 |
| | | | GCGAAGGCTG | |
| GATA3 | GATA3 | 3′ | TCTGCAGCCAGGAGAGCAGGGACTCCAACCCTTAGGGAACCC | 100 |
| GZMB | GRB | 5′ | GTGCCAGCAAGATCCAATCTAGANNNNNNNAACTTCTCCAACGACA | 101 |
| | | | TCATGCTACTGCAG | |
| GZMB | GRB | 3′ | CTGGAGAGAAAGGCCAAGCGGACCAGTCCAACCCTTAGGGAACCC | 102 |
| HBZ | HTLV1 | 5′ | GTGCCAGCAAGATCCAATCTAGANNNNNNNCCTGGCGGCCTCAGG | 103 |
| | | | GCTGTTTCGATGCTTGCCTGTGTCATGCC | |
| HBZ | HTLV1 | 3′ | CGGAGGACCTGCTGGTGGAGGAATTGGTGGACGGGCTATTATTCC | 104 |
| | | | AACCCTTAGGGAACCC | |
| ICOS | ICOS | 5′ | GTGCCAGCAAGATCCAATCTAGANNNNNNNAAAGTAACTCTTACAG | 105 |
| | | | GAGGATATTTGCATATTTATG | |
| ICOS | ICOS | 3′ | AATCACAACTTTGTTGCCAGCTGAAGTTCTGTCCAACCCTTAGGGA | 106 |
| | | | ACCC | |
| IDH2 | IDH2R1 | 5′ | GTGCCAGCAAGATCCAATCTAGANNNNNNNCCAAGCCCATCACCA | 107 |
| | 72K | | TTGGCAA | |
| IDH2 | IDH2R1 | 5′ | GTGCCAGCAAGATCCAATCTAGANNNNNNNCCAAGCCCATCACCA | 108 |
| | 72T | | TTGGCAC | |
| IDH2 | IDH2R1 | 3′ | GCACGCCCATGGCGACCAGTTCCAACCCTTAGGGAACCC | 109 |
| | 72 | | | |
| IFNG | INFg | 5′ | GTGCCAGCAAGATCCAATCTAGANNNNNNNAACGAGATGACTTCG | 110 |
| | | | AAAAGCTGACTAATTATTCG | |
| IFNG | INFg | 3′ | GTAACTGACTTGAATGTCCAACGCAAAGCATCCAACCCTTAGGGAA | 111 |
| | | | CCC | |
| IGH | JH | 5′ | GTGCCAGCAAGATCCAATCTAGANNNNNNNGCACCCTGGTCACCG | 112 |
| | | | TCTCCTCAG | |
| IGH | Imu | 5′ | GTGCCAGCAAGATCCAATCTAGANNNNNNNAGTGACCAGGCGCCC | 113 |
| | | | GACATG | |
| IGH | Igamma | 5 | GTGCCAGCAAGATCCAATCTAGANNNNNNNCTCTCAGCCAGGACC | 114 |
| | | | AAGGACAGCAG | |
| IGH | lalpha | 5′ | GTGCCAGCAAGATCCAATCTAGANNNNNNNGCCCTCCAGCAGCCT | 115 |
| | | | GACCAG | |
| IGH | lepsilon | 5′ | GTGCCAGCAAGATCCAATCTAGANNNNNNNCAAATGGACGACCCG | 116 |
| | | | GTGCTTCAG | |
| IGH | Cmu | 3′ | GGAGTGCATCCGCCCCAACCTCCAACCCTTAGGGAACCC | 117 |
| IGH | Cgamma | 3, | CTTCCACCAAGGGCCCATCGGTTCCAACCCTTAGGGAACCC | 118 |
| IGH | Calpha | 3′ | CATCCCCGACCAGCCCCAAGTCCAACCCTTAGGGAACCC | 119 |
| IGH | Cepsilon | 3′ | CCTCCACACAGAGCCCATCCGTCTTTCCAACCCTTAGGGAACCC | 120 |
| IGHD | IGHD | 5′ | GTGCCAGCAAGATCCAATCTAGANNNNNNNGTTGATGGCGCTGAG | 121 |
| | | | AGAACCCG | |
| IGHD | IGHD | 3′ | CTGCGCAGGCACCCGTCAAGTCCAACCCTTAGGGAACCC | 122 |
| IGHM | IGHM | 5′ | GTGCCAGCAAGATCCAATCTAGANNNNNNNGCGTCCTCCATGTGT | 123 |
| | | | GGCCCCG | |
| IGHM | IGHM | 3′ | ATCAAGACACAGCCATCCGGGTCTTCTCCAACCCTTAGGGAACCC | 124 |
| IL411 | IL411 | 5′ | GTGCCAGCAAGATCCAATCTAGANNNNNNNAGGTGCTCAGCGATG | 125 |
| | | | CTGGACACAAG | |
| IL411 | IL411 | 3′ | GTCACCATCCTGGAGGCAGATAACAGGATCTCCAACCCTTAGGGA | 126 |
| | | | ACCC | |
| IRF4 | IRF4 | 5′ | GTGCCAGCAAGATCCAATCTAGANNNNNNNCTGCCGAAGCCTTGG | 127 |
| | | | CGTTCTCAG | |
| IRF4 | IRF4 | 3′ | ACTGCCGGCTGCACATCTGCCTGTATCCAACCCTTAGGGAACCC | 128 |
| ITPKB | ITPKB | 5′ | GTGCCAGCAAGATCCAATCTAGANNNNNNNGGATCCAGCTGGCAG | 129 |
| | | | GACACGCAG | |
| ITPKB | ITPKB | 3′ | GGAGTTTCAAGGCAGCTGCCAATGGCATCCAACCCTTAGGGAACC | 130 |
| | | | C | |
| JAK2 | JAK2 | 5′ | GTGCCAGCAAGATCCAATCTAGANNNNNNNCAAGACCAGATGGAT | 131 |
| | | | GCCCAGATGAG | |
| JAK2 | JAK2 | 3′ | ATCTATATGATCATGACAGAATGCTGGAACTCCAACCCTTAGGGAA | 132 |
| | | | CCC | |
| LAG3 | LAG3 | 5′ | GTGCCAGCAAGATCCAATCTAGANNNNNNNAGCCGCTTTGGGTGG | 133 |
| | | | CTCCAG | |
| LAG3 | LAG3 | 3′ | TGAAGCCTCTCCAGCCAGGGGTCCAACCCTTAGGGAACCC | 134 |
| LIMD1 | LIMD1 | 5′ | GTGCCAGCAAGATCCAATCTAGANNNNNNNTTTCTTTGTGGACATC | 135 |
| | | | TGATCATGGACATG | |
| LIMD1 | LIMD1 | 3′ | ATCCTGCAAGCCCTGGGGAAGTCCTACCTCCAACCCTTAGGGAAC | 136 |
| | | | CC | |
| LMO2 | LMO2 | 5′ | GTGCCAGCAAGATCCAATCTAGANNNNNNNCGGAAGCTCTGCCGG | 137 |
| | | | AGAGACTATCTCAG | |
| LMO2 | LMO2 | 3 | GCTTTTTGGGCAAGACGGTCTCTGCTCCAACCCTTAGGGAACCC | 138 |
| MAL | MAL | 5′ | GTGCCAGCAAGATCCAATCTAGANNNNNNNGGTGGAGAGACTTCC | 139 |
| | | | TGGGTCACCTTG | |
| MAL | MAL | 3′ | GACGCAGCCTACCACTGCACCGTCCAACCCTTAGGGAACCC | 140 |
| MAML3 | MAML3 | 5′ | GTGCCAGCAAGATCCAATCTAGANNNNNNNCTTACGCTGCACTTCC | 141 |
| | | | ATCCCACGGTCAG | |
| MAML3 | MAML3 | 3′ | GAGCAGCATCCAGTTGGACTTCCCCGAATCCAACCCTTAGGGAAC | 142 |
| | | | CC | |
| MEF2B | MEF2B | 5′ | GTGCCAGCAAGATCCAATCTAGANNNNNNNCAACACTGACATCCTC | 143 |
| | | | GAGGTACCCCAG | |
| MEF2B | MEF2B | 3′ | ACGCTGAAGCGGAGGGGCATTTCCAACCCTTAGGGAACCC | 144 |
| MKI67 | KI67 | 5′ | GTGCCAGCAAGATCCAATCTAGANNNNNNNTCCCCTGAGCCTCAG | 145 |
| | | | CACCTGCTTGTTTGGAAG | |
| MKI67 | KI67 | 3′ | GGGTATTGAATGTGACATCCGTATCCAGCTTCCTGTTGTCCAACCC | 146 |
| | | | TTAGGGAACCC | |
| MME | CD10 | 5′ | GTGCCAGCAAGATCCAATCTAGANNNNNNNTACAAGGAGTCCAGA | 147 |
| | | | AATGCTTTCCGCAAG | |
| MME | CD10 | 3′ | GCCCTTTATGGTACAACCTCAGAAACAGCATCCAACCCTTAGGGAA | 148 |
| | | | CCC | |
| MS4A1 | MS4A1 | 5′ | GTGCCAGCAAGATCCAATCTAGANNNNNNNTTCTTCATGAGGGAAT | 149 |
| | | | CTAAGACTTTGGGG | |
| MS4A1 | MS4A1 | 3′ | GCTGTCCAGATTATGAATGGGCTCTTCCACTCCAACCCTTAGGGAA | 150 |
| | | | CCC | |
| MYBL1 | MYBL1 | 5′ | GTGCCAGCAAGATCCAATCTAGANNNNNNNCCAGAATTTGCAGAG | 151 |
| | | | ACTCTAGAACTTATTGAATCT | |
| MYBL1 | MYBL1 | 3′ | GATCCTGTAGCATGGAGTGACGTTACCAGTTTTTCCAACCCTTAGG | 152 |
| | | | GAACCC | |
| MYC | MYC | 5′ | GTGCCAGCAAGATCCAATCTAGANNNNNNNTCGGGTAGTGGAAAA | 153 |
| | | | CCAGCAGCCTC | |
| MYC | MYC | 5′ | GTGCCAGCAAGATCCAATCTAGANNNNNNNCCCACCACCAGCAGC | 154 |
| | | | GACTCTG | |
| MYC | MYC | 3′ | CCGCGACGATGCCCCTCAACGTTATCCAACCCTTAGGGAACCC | 155 |
| MYC | MYC | 3′ | AGGAGGAACAAGAAGATGAGGAAGAAATCGTCCAACCCTTAGGGA | 156 |
| | | | ACCC | |
| MYD88 | MYD88 | 5′ | GTGCCAGCAAGATCCAATCTAGANNNNNNNCAGGTGCCCATCAGA | 157 |
| | | | AGCGACC | |
| MYD88 | MYD88 | 5′ | GTGCCAGCAAGATCCAATCTAGANNNNNNNGTCTATTGCTAGTGAG | 158 |
| | | | CTCATCGAAAAGAG | |
| MYD88 | MYD88 | 3′ | GATCCCCATCAAGTACAAGGCAATGAAGAATCCAACCCTTAGGGAA | 159 |
| | | | CCC | |
| MYD88 | MYD88 | 3′ | GTGCCGCCGGATGGTGGTGGTCCAACCCTTAGGGAACCC | 160 |
| NCAM1 | CD56 | 5′ | GTGCCAGCAAGATCCAATCTAGANNNNNNNCACCCCCTCTGCCAG | 161 |
| | | | CTATCTGGAG | |
| NCAM1 | CD56 | 3′ | GTGACCCCAGACTCTGAGAATGATTTTGGTCCAACCCTTAGGGAAC | 162 |
| | | | CC | |
| NEK6 | NEK6 | 5′ | GTGCCAGCAAGATCCAATCTAGANNNNNNNCCTGTGCATCCTCCT | 163 |
| | | | GACCCACAG | |
| NEK6 | NEK6 | 3′ | AGGCATCCCAACACGCTGTCTTTTCCAACCCTTAGGGAACCC | 164 |
| PDCD1 | PD1 | 5′ | GTGCCAGCAAGATCCAATCTAGANNNNNNNCGGGCAGAGCTCAGG | 165 |
| | | | GTGACAG | |
| PDCD1 | PD1 | 3′ | AGAGAAGGGCAGAAGTGCCCACAGCTCCAACCCTTAGGGAACCC | 166 |
| PDCD1 | PDL2 | 5′ | GTGCCAGCAAGATCCAATCTAGANNNNNNNAACTTACTTTGGCCAG | 167 |
| LG2 | | | CATTGACCTTCAAA | |
| PDCD1 | PDL2 | 3′ | GTCAGATGGAACCCAGGACCCATCCTCCAACCCTTAGGGAACCC | 168 |
| LG2 | | | | |
| PIM2 | PIM2 | 5′ | GTGCCAGCAAGATCCAATCTAGANNNNNNNACACCGCCTCACAGA | 169 |
| | | | TCGACTCCAG | |
| PIM2 | PIM2 | 3′ | GTGGCCATCAAAGTGATTCCCCGTCCAACCCTTAGGGAACCC | 170 |
| PRDM1 | PRDM1 | 5′ | GTGCCAGCAAGATCCAATCTAGANNNNNNNACTTTCGGCCAGCTC | 171 |
| | | | TCCAATCTGAAG | |
| PRDM1 | PRDM1 | 3′ | GTCCACCTGAGAGTGCACAGTGGAGAACTCCAACCCTTAGGGAAC | 172 |
| | | | CC | |
| PRF1 | PRF | 5′ | GTGCCAGCAAGATCCAATCTAGANNNNNNNACACGGTGGAGTGCC | 173 |
| | | | GCTTCTACAG | |
| PRF1 | PRF | 3′ | TTTCCATGTGGTACACACTCCCCCGTCCAACCCTTAGGGAACCC | 174 |
| PTPRC | CD45R | 5′ | GTGCCAGCAAGATCCAATCTAGANNNNNNNAAAGCCCAACACCTT | 175 |
| | O | | CCCCCACTG | |
| PTP RC | CD45R | 3′ | ATGCCTACCTTAATGCCTCTGAAACAACCATCCAACCCTTAGGGAA | 176 |
| | O | | CCC | |
| RAB29 | RAB7L | 5′ | GTGCCAGCAAGATCCAATCTAGANNNNNNNCGGCTTCAGCTGTGG | 177 |
| | 1 | | GATATTGCAG | |
| RAB29 | RAB7L | 3′ | GGCAGGAGCGCTTCACCTCTATGACATCCAACCCTTAGGGAACCC | 178 |
| | 1 | | | |
| RHOA | RHOA | 5′ | GTGCCAGCAAGATCCAATCTAGANNNNNNNGGTGATTGTTGGTGA | 179 |
| | G17V | | TGGAGCCTGTGT | |
| RHOA | RHOA | 3′ | AAAGACATGCTTGCTCATAGTCTTCAGCAAGGACCTCCAACCCTTA | 180 |
| | G17V | | GGGAACCC | |
| S1PR2 | S1PR2 | 5′ | GTGCCAGCAAGATCCAATCTAGANNNNNNNAGCCGGGCCGGCCTA | 181 |
| | | | GCCAG | |
| S1PR2 | S1PR2 | 3′ | TTCTGAAAGCCCCATGGCCCCTCCAACCCTTAGGGAACCC | 182 |
| SDC1 | CD138 | 5′ | GTGCCAGCAAGATCCAATCTAGANNNNNNNAGCAGAGGGCTCTGG | 183 |
| | | | GGAGCAG | |
| SDC1 | CD138 | 3′ | GACTTCACCTTTGAAACCTCGGGGGAGTCCAACCCTTAGGGAACC | 184 |
| | | | C | |
| SERPI | SERPI | 5′ | GTGCCAGCAAGATCCAATCTAGANNNNNNNGGCAGGAGAAGAGGA | 185 |
| NA9 | NA9 | | ACCTGCAAAG | |
| SERPI | SERPI | 3′ | ACATATTTTGTTCCAAAATGGCATCTTACCTCCAACCCTTAGGGAAC | 186 |
| NA9 | NA9 | | CC | |
| SH3BP | SH3BP | 5′ | GTGCCAGCAAGATCCAATCTAGANNNNNNNCTCAAGGCAAAGTAC | 187 |
| 5 | 5 | | TATGTGCAGCTCGAG | |
| SH3BP | SH3BP | 3′ | CAACTGAAAAAGACTGTGGATGACCTGCAGTCCAACCCTTAGGGAA | 188 |
| 5 | 5 | | CCC | |
| STAT6 | STAT6 | 5′ | GTGCCAGCAAGATCCAATCTAGANNNNNNNCTGCTAATGGGACTG | 189 |
| | | | GGCCAAGTGAG | |
| STAT6 | STAT6 | 3′ | GCCCTGGCCATGCTACTGCAGGTCCAACCCTTAGGGAACCC | 190 |
| TBX21 | TBET | 5′ | GTGCCAGCAAGATCCAATCTAGANNNNNNNCCAAAGGATTCCGGG | 191 |
| | | | AGAACTTTGAGTC | |
| TBX21 | TBET | 3′ | CATGTACACATCTGTTGACACCAGCATCCCTCCAACCCTTAGGGAA | 192 |
| | | | CCC | |
| TCL1A | TCL1A | 5′ | GTGCCAGCAAGATCCAATCTAGANNNNNNNCAGTTTCTGGCGCTTA | 193 |
| | | | GTGTACCACATCAAG | |
| TCL1A | TCL1A | 3′ | ATTGACGGCGTGGAGGACATGCTTTCCAACCCTTAGGGAACCC | 194 |
| TFRC | CD71 | 5′ | GTGCCAGCAAGATCCAATCTAGANNNNNNNGCACAGACTTCACCG | 195 |
| | | | GCACCATCAA | |
| TFRC | CD71 | 3′ | GCTGCTGAATGAAAATTCATATGTCCCTCGTCCAACCCTTAGGGAA | 196 |
| | | | CCC | |
| TNFRS | TACI | 5′ | GTGCCAGCAAGATCCAATCTAGANNNNNNNGCGCACCTGTGCAGC | 197 |
| F13B | | | CTTCTGCA | |
| TNFRS | TACI | 3′ | GGTCACTCAGCTGCCGCAAGGAGCTCCAACCCTTAGGGAACCC | 198 |
| F13B | | | | |
| TNFRS | BCMA | 5′ | GTGCCAGCAAGATCCAATCTAGANNNNNNNCTCTAACATGTCAGC | 199 |
| F17 | | | GTTATTGTAATGCAA | |
| TNFRS | BCMA | 3′ | GTGTGACCAATTCAGTGAAAGGAACGTCCAACCCTTAGGGAACCC | 200 |
| F17 | | | | |
| TNFRS | CD30 | 5′ | GTGCCAGCAAGATCCAATCTAGANNNNNNNTGTACAGCCTGCGTG | 201 |
| F8 | | | ACTTGTTCTCGAG | |
| TNFRS | CD30 | 3′ | ACGACCTCGTGGAGAAGACGCCGTCCAACCCTTAGGGAACCC | 202 |
| F8 | | | | |
| TNFSF | APRIL | 5′ | GTGCCAGCAAGATCCAATCTAGANNNNNNNGTTCCCATTAACGCCA | 203 |
| 13 | | | CCTCCAAGG | |
| TNFSF | APRIL | 3′ | ATGACTCCGATGTGACAGAGGTGATGTGTCCAACCCTTAGGGAAC | 204 |
| 13 | | | CC | |
| TNFSF | BAFF | 5′ | GTGCCAGCAAGATCCAATCTAGANNNNNNNAGCTGTCACCGCGGG | 205 |
| 13B | | | ACTGAAA | |
| TNFSF | BAFF | 3′ | ATCTTTGAACCACCAGCTCCAGGAGAAGTCCAACCCTTAGGGAACC | 206 |
| 13B | | | C | |
| TRA | TRAC | 5′ | GTGCCAGCAAGATCCAATCTAGANNNNNNNCTGCGGCTGTGGTCC | 207 |
| | | | AGCTGAG | |
| TRA | TRAC | 3′ | ATCTGCAAGATTGTAAGACAGCCTGTGCTCTCCAACCCTTAGGGAA | 208 |
| | | | CCC | |
| TRAF1 | TRAF1 | 5′ | GTGCCAGCAAGATCCAATCTAGANNNNNNNGCAGGCTGTCTCTCT | 209 |
| | | | GAGAACCCGAG | |
| TRAF1 | TRAF1 | 3′ | GAATGGCGAGGATCAGATCTGCCCCTCCAACCCTTAGGGAACCC | 210 |
| TRB | TCRbeta | 5′ | GTGCCAGCAAGATCCAATCTAGANNNNNNNGCCGAGGCCTGGGGT | 211 |
| | | | AGAGCAG | |
| TRB | TCRbeta | 3′ | ACTGTGGCTTCACCTCCGAGTCTTACCATCCAACCCTTAGGGAACC | 212 |
| | | | C | |
| TRD | TCRdelta | 5′ | GTGCCAGCAAGATCCAATCTAGANNNNNNNCTGACTTTGAAGTGAA | 213 |
| | | | GACAGATTCTACAG | |
| TRD | TCRdelta | 3′ | ATCACGTAAAACCAAAGGAAACTGAAAACACTCCAACCCTTAGGGA | 214 |
| | | | ACCC | |
| TRG | TCRgamma | 5′ | GTGCCAGCAAGATCCAATCTAGANNNNNNNAAGAAATTATCTTTCC | 215 |
| | | | TCCAATAAAGACAG | |
| TRG | TCRgamma | 3′ | ATGTCATCACAATGGATCCCAAAGACAATTTCCAACCCTTAGGGAA | 216 |
| | | | CCC | |
| XBP1 | XBP1 | 5′ | GTGCCAGCAAGATCCAATCTAGANNNNNNNTCTGGCGGTATTGAC | 217 |
| | | | TCTTCAGATTCAGAG | |
| XBP1 | XBP1 | 3′ | TCTGATATCCTGTTGGGCATTCTGGACAACTCCAACCCTTAGGGAA | 218 |
| | | | CCC | |
| XPO1 | XPOE5 | 5′ | GTGCCAGCAAGATCCAATCTAGANNNNNNNTTCTGAAGACTGTAGT | 219 |
| | 71K | | TAACAAGCTGTTCA | |
| XPO1 | XPOW | 5′ | GTGCCAGCAAGATCCAATCTAGANNNNNNNACTATTATTTGTGATC | 220 |
| | T | | TTCAGCCTCAACAG | |
| XPO1 | XPOE5 | 3′ | AATTCATGCATGAGACCCATGATGGAGTCTCCAACCCTTAGGGAAC | 221 |
| | 71K | | CC | |
| XPO1 | XPOW | 3′ | GTTCATACGTTTTATGAAGCTGTGGGGTACTCCAACCCTTAGGGAA | 222 |
| | T | | CCC | |
| ZAP70 | ZAP70 | 5′ | GTGCCAGCAAGATCCAATCTAGANNNNNNNGCAGACCGACGGCAA | 223 |
| | | | GTTCCT | |
| ZAP70 | ZAP70 | 3′ | GCTGAGGCCGCGGAAGGAGCTCCAACCCTTAGGGAACCC | 224 |

### Example 2

Methodology

900 biopsies samples including B-cells NHL but also other lymphoma subtypes and biopsy samples were used to train the assay, which included 31 Hodgkin lymphomas, 578 B-cells lymphoma, 253 T-cells lymphomas, and 38 non-tumor controls. For each biopsy, RNA were extracted and the expression levels of 137 RNA markers (see below) were analyzed using a dedicated RT-MLPA assay. The set of markers include B cells markers (CD19, CD22, MS4A1 encoding for (e.g., CD20), T cells markers (e.g., CD3, CD5, CD8) with markers of the Th1/Th2 polarization (e.g., IFN-gamma, TBET, PRF, GRB, CXCR5, CXCL13, ICOS, GATA3, FOXP3) and macrophages markers (e.g., CD68, CD163). The assay was also designed to evaluate the expression of RNA markers differentially expressed in the 3 most frequent DLBCL subtypes (ABC, GCB and PMBL), to detect recurrent somatic variants MYD88L265P, RHOAG17V and BRAFV600E, to evaluate the expression of prognostic markers (e.g., MYC, BCL2, BCL6, Ki67), of therapeutic targets (e.g., CD19, CD20, CD30, CRBN,) and to detect some viral infections (EBV and HTLV-1). Markers involved in immune checkpoint and anti-tumor immune response like PD1, PD-L1, PD-L2 and CTLA-4 were also employed. Finally, markers involved in immunoglobulin class switching and somatic hypermutation were included (AICDA, surface immunoglobulin).

The aforementioned set of 137 markers is:

AIDe2-AIDe3, AIDe4-AIDe5, ALK, ANXA1, APRIL, ASB13, B2M, BAFF, BANK, BCL2e1b-BCL2e2b, BCL2e1-BCL2e2, BCL6e1-BCL6e2, BCL6e1-Calpha, BCL6e1-Cepsilon, BCL6e1-C-gamma, BCL6e1-Cmu, BCL6e3-BCL6e4, BCMA, BRAFV600E, CARD11, CCDC50, CCND1, CCND2, CCR4, CCR7, CD10, CD138, CD163, CD19, CD22, CD23, CD27, CD28, CD3, CD30, CD38, CD4, CD40, CD40Le2-CD40Le3, CD40Le3-CD40Le4, CD45RO, CD5, CD56, CD68, CD70, CD71, CD8, CD80, CD86, CD95, CRBN, CREB3L2, CTLA4, CXCL13, CXCR5, CYB5R2, DUSP22, EBER1, FGFR1, FOXP1, FOXP3, GATA3, GRB, HTLV1, I-alpha-BCL6e2, I-alpha-C-alpha, I-alpha-C-epsilon, I-alpha-C-gamma, I-alpha-C-mu, ICOS, IDH2R172K, IDH2R172T, Iepsilon-BCL6e2, I-epsilon-C-alpha, I-epsilon-C-epsilon, I-epsilon-C-gamma, I-epsilon-C-mu, I-gamma-BCL6e2, I-gamma-C-alpha, I-gamma-C-epsilon, I-gamma-C-gamma, I-gamma-C-mu, IGHD, IGHM, IL4I1, I-mu-BCL6e2, I-mu-C-alpha, I-mu-C-epsilon, I-mu-C-gamma, I-mu-C-mu, INFg, IRF4, ITPKB, JAK2, JH-BCL6e2, JH-C-alpha, JH-C-epsilon, JH-C-gamma, JH-C-mu, KI67, LAGS, LIMD1, LMO2, MAL, MAML3, MEF2B, MS4A1, MYBL1, MYCe1-MYCe2, MYCe2-MYCe3, MYD88e3-MYD88e4, MYD88L265P, NEK6, PD1, PDL1, PDL2, PIM2, PRDM1, PRF, RAB7L1, RHOAG17V, S1PR2, SERPINA9, SH3BP5, STAT6, TACI, TBET, TCL1A, TCR-beta, TCR-delta, TCR-gamma, TRAC (TCR-alpha), TRAF1, XBP1, XPOE571K, XPOWT, and ZAP70.

For this assay, RNA samples were first converted into cDNA by reverse transcription. Those cDNA were next incubated with a mixture of 224 oligonucleotide probes binding at the extremities of exons of the targeted RNA markers and harboring additional tails (Table XVII). After this incubation step, those probes hybridized at the extremities of adjacent exons were ligated by the adjunction of a DNA ligase, and amplified by PCR using primers corresponding to the additional tails, and allowing their analysis on a next generation sequencer. PCR products were purified and loaded onto a flow cell. Sequencing reads are de-multiplexed using the index sequences introduced during PCR amplification, aligned with the sequences of the probes and counted. All results are normalized according to the UMI sequences to avoid PCR amplification bias.

The gene expression levels of the 137 markers (see table XVII) were evaluated using precise counting of sequences of interest after UMI (Unique Molecular Identifiers) data processing, avoiding bias of amplification. Samples with more than 5000 reads with different UMIs were considered interpretable.

The inventors next trained a machine learning based random forest (RF) algorithm for classification. See accompanying electronic table entitled database.txt, created on Mar. 28, 2018 for data for training.

This algorithm of classification first relies on four independent algorithms:

The first to discriminate B cells-lymphomas (LNH_B) from T-cells lymphomas (LNH_T), Trained on 578 B-Cells lymphomas and 253 T-Cells lymphomas).

The second to discriminate High grade (DLBCL) from low grade (Small cells) B-Cells lymphomas, trained on 429 and 109 samples respectively.

The third to discriminate the three main gene expression signatures observed in B-cells lymphomas (Activated B-Cell (ABC), 262 cases; Germinal Centre B-cell (GCB), 204 cases; Primary Mediastinal B-cell (PMBL), 46 cases).

The fourth to discriminate the three main gene expression signatures observed in T-cells lymphomas (T-cytotoxic, 45 cases; T-follicular helper, 116 cases; T-helper2, 32 cases).

The algorithm also relies on a fifth, global algorithm, trained to recognize 16 different categories of samples, including non-tumor reactive biopsies and 15 lymphoma diagnosis:

Small Lymphocytic lymphomas (SLL, 19 cases)

Natural Killer T-cells Lymphomas (NKTCL, 12 cases)

Marginal Zone Lymphomas (MZL, 40 cases)

Mantle Cells lymphomas (MCL, 34 cases)

Hodgkin Lymphomas (Hodgkin, 31 cases)

Follicular Lymphomas (FL, 50 cases)

Primary Mediastinal B Cell Lymphomas (DLBCL_PMBL, 46 cases)

GCB Diffuse large B cells lymphomas (DLBCL_GCB, 165 cases)

EBV positive Diffuse large B cells lymphomas (DLBCL_EBV, 11 cases)

ABC Diffuse large B cells lymphomas (DLBCL_ABC, 167 cases)

Adult T-cells Leukemia/Lymphoma (ATLL, 8 cases)

ALK positive anaplastic large cells Lymphomas (ALCL_ALK+, 15 cases)

ALK negative anaplastic large cells Lymphomas, cytotoxic (ALCL_ALK−, 18 cases)

ALK negative anaplastic large cells Lymphomas, non-cytotoxic (ALCL_ALK−_Cn, 24 cases)

Angioimmunoblastic T-cells lymphomas (AITL, 103 cases)

Reactive, non-tumor biopsies (Reactive, 38 cases)

The out of bag scores (00B) obtained during the training of the 5 algorithms, which evaluate the accuracy of the prediction algorithms indicate that:

The first can discriminate B cells-lymphomas (LNH_B) from T-cells lymphomas (LNH_T) with a precision greater than 97.1%.

The second can discriminate High grade (DLBCL) from low grade (Small cells) B-Cells lymphomas with a precision greater than 92.6%.

The third can discriminate the three main gene expression signatures observed in B-cells lymphomas with a precision greater than 96.9%.

The fourth can discriminate the three main gene expression signatures observed in T-cells lymphomas with a precision greater than 90.7%.

The fifth can classify the sample into one of the 16 categories with a precision of more than 86%.

### Example 3

To calculate scores for the markers, the inventors used trained a random forest model on Python, using the SKLEARN package with the RandomForestClassifier function. They next used the <<feature_importance>> attribute, which returned a coefficient for each of the markers.

This coefficient is a function of the «weight» of the genes in the final model, which increases when the genes are selected in the trees, and used «tall». This is what it gives regarding the classification of 137 markers. The right column, which ranks the importance of each marker, corresponds to the coefficients. The higher they are, the more weight the marker has in the overall model. Table XIII lists the marks as ranked and with the relative importance indicated.

TABLE XIII

| Rank | Marker | Importance |
|---|---|---|
| 1 | CYB5R2 | 0.03026645 |
| 2 | LIMD1 | 0.03023021 |
| 3 | CD10 | 0.02985653 |
| 4 | PDL2 | 0.02839509 |
| 5 | CCND1 | 0.02697442 |
| 6 | TACI | 0.02681505 |
| 7 | IRF4 | 0.02545914 |
| 8 | SERPINA9 | 0.02526377 |
| 9 | MYBL1 | 0.02187064 |
| 10 | CCND2 | 0.02168564 |
| 11 | S1PR2 | 0.02145768 |
| 12 | CD40Le2-CD40Le3 | 0.02032691 |
| 13 | PIM2 | 0.01888269 |
| 14 | CREB3L2 | 0.01486954 |
| 15 | NEK6 | 0.01464888 |
| 16 | MAML3 | 0.01439519 |
| 17 | Imu-Cmu | 0.01276586 |
| 18 | RAB7L1 | 0.0125856 |
| 19 | FOXP1 | 0.01244864 |
| 20 | PDL1 | 0.01238951 |
| 21 | CD27 | 0.01212423 |
| 22 | ICOS | 0.01204473 |
| 23 | CD23 | 0.01197463 |
| 24 | IGHM | 0.01191564 |
| 25 | IL4I1 | 0.0119101 |
| 26 | LMO2 | 0.01134336 |
| 27 | KI67 | 0.01086805 |
| 28 | JAK2 | 0.01066631 |
| 29 | CD71 | 0.01051425 |
| 30 | CD68 | 0.01026072 |
| 31 | ASB13 | 0.00971372 |
| 32 | TCL1A | 0.00944097 |
| 33 | BANK | 0.00910599 |
| 34 | CD5 | 0.00909347 |
| 35 | CD30 | 0.00866066 |
| 36 | CCDC50 | 0.00866001 |
| 37 | CD28 | 0.00860346 |
| 38 | BCL6e1-BCL6e2 | 0.00850226 |
| 39 | BCL6e3-BCL6e4 | 0.00841083 |
| 40 | CD163 | 0.00835908 |
| 41 | SH3BP5 | 0.00832826 |
| 42 | CD22 | 0.00827696 |
| 43 | MAL | 0.00819158 |
| 44 | CARD11 | 0.0080844 |
| 45 | ITPKB | 0.00796354 |
| 46 | XBP1 | 0.00772687 |
| 47 | AIDe2-AIDe3 | 0.00755497 |
| 48 | CCR7 | 0.00736932 |
| 49 | Igamma-Cgamma | 0.0073285 |
| 50 | AIDe4-AIDe5 | 0.00695632 |
| 51 | GRB | 0.00671764 |
| 52 | GATA3 | 0.00664773 |
| 53 | Iepsilon-Cepsilon | 0.00600629 |
| 54 | CXCR5 | 0.00566252 |
| 55 | BAFF | 0.00532812 |
| 56 | ZAP70 | 0.00525757 |
| 57 | PRDM1 | 0.00492013 |
| 58 | TBET | 0.00476811 |
| 59 | TRAF1 | 0.00473835 |
| 60 | CD95 | 0.00470593 |
| 61 | JH-Cmu | 0.00454466 |
| 62 | CXCL13 | 0.00452055 |
| 63 | MYCe1-MYCe2 | 0.00443664 |
| 64 | CD138 | 0.00442926 |
| 65 | TCRbeta | 0.00427502 |
| 66 | BCL2e1-BCL2e2 | 0.0041906 |
| 67 | MEF2B | 0.00404202 |
| 68 | TRAC | 0.00403151 |
| 69 | PRF | 0.0038721 |
| 70 | MS4A1 | 0.00383217 |
| 71 | FOXP3 | 0.00378571 |
| 72 | CRBN | 0.00374515 |
| 73 | CD38 | 0.00370072 |
| 74 | CD70 | 0.00364833 |
| 75 | JH-Cgamma | 0.00359519 |
| 76 | CD56 | 0.00351585 |
| 77 | INFg | 0.00351559 |
| 78 | CCR4 | 0.00349336 |
| 79 | CTLA4 | 0.00348812 |
| 80 | LAG3 | 0.00329335 |
| 81 | CD19 | 0.00329085 |
| 82 | BCMA | 0.00326716 |
| 83 | STAT6 | 0.00321652 |
| 84 | Ialpha-Calpha | 0.00321181 |
| 85 | CD86 | 0.00318868 |
| 86 | CD80 | 0.0031832 |
| 87 | B2M | 0.00313425 |
| 88 | JH-Cepsilon | 0.00312053 |
| 89 | BCL2e1b-BCL2e2b | 0.00310219 |
| 90 | CD4 | 0.00307523 |
| 91 | CD3 | 0.00306732 |
| 92 | IGHD | 0.00303654 |
| 93 | ANXA1 | 0.00301974 |
| 94 | Igamma-Cepsilon | 0.00281775 |
| 95 | APRIL | 0.00277334 |
| 96 | FGFR1 | 0.00274478 |
| 97 | CD8 | 0.00251412 |
| 98 | MYD88e3-MYD88e4 | 0.00248746 |
| 99 | Imu-Calpha | 0.0024821 |
| 100 | XPOWT | 0.00238902 |
| 101 | CD45RO | 0.00238321 |
| 102 | MYCe2-MYCe3 | 0.00236764 |
| 103 | PD1 | 0.00232968 |
| 104 | CD40 | 0.00224707 |
| 105 | DUSP22 | 0.00222888 |
| 106 | TCRgamma | 0.00216243 |
| 107 | TCRdelta | 0.00213625 |
| 108 | Imu-Cgamma | 0.00206404 |
| 109 | JH-Calpha | 0.00200654 |
| 110 | MYD88L265P | 0.00172309 |
| 111 | RHOAG17V | 0.00116103 |
| 112 | Imu-Cepsilon | 0.00115879 |
| 113 | Igamma-Calpha | 0.00099066 |
| 114 | CD40Le3-CD40Le4 | 0.00096684 |
| 115 | ALK | 0.00084062 |
| 116 | Iepsilon-Calpha | 0.0007831 |
| 117 | XPOE571K | 0.00071954 |
| 118 | EBER1 | 0.0006997 |
| 119 | Igamma-Cmu | 0.00055874 |
| 120 | Iepsilon-Cgamma | 0.00054491 |
| 121 | BRAFV600E | 0.00047387 |
| 122 | Ialpha-Cmu | 0.00034858 |
| 123 | Imu-BCL6e2 | 0.00028369 |
| 124 | JH-BCL6e2 | 0.0002229 |
| 125 | BCL6e1-Cmu | 0.000193 |
| 126 | BCL6e1-Cepsilon | 0.00016948 |
| 127 | Ialpha-Cgamma | 0.00014921 |
| 128 | IDH2R172K | 0.00013304 |
| 129 | BCL6e1-Cgamma | 0.00013026 |
| 130 | Ialpha-Cepsilon | 8.57E−05 |
| 131 | Iepsilon-BCL6e2 | 8.06E−05 |
| 132 | BCL6e1-Calpha | 2.27E−05 |
| 133 | Igamma-BCL6e2 | 1.94E−05 |
| 134 | Iepsilon-Cmu | 1.62E−05 |
| 135 | Ialpha-BCL6e2 | 0 |
| 136 | IDH2R172T | 0 |
| 137 | HTLV1 | 0 |

1. A gene expression assay kit for distinguishing subtypes of B-cell non-Hodgkin Lymphoma comprising a set of probes that is capable of distinguishing among Activated B-cell Diffuse Large B-cell Lymphoma (ABC DLBCL), Germinal Center B-cell like Diffuse Large B-cell Lymphoma (GCB DLBCL), Primary Mediastinal large B-cell Lymphoma (PMBL), Follicular Lymphoma (FL), Mantle Cell Lymphoma (MCL), Small Lymphocytic Lymphoma (SLL) and Marginal Cell Lymphoma (MZL), wherein the set of probes is capable of detecting the RNA expression of at least one marker from tumor cells of a lymphoma and at least one marker from bystander non-tumor cells located in a microenvironment of said lymphoma.

2. The gene expression assay kit according to claim 1, wherein the set of probes is capable of detecting RNA expression of TACI, CCND1, CD10, CD30, MAL, LMO2, CD5, CD23, CD28, ICOS, and CTLA4.

3. The gene expression assay kit according to claim 1, wherein the assay kit comprises a pair of probes for detecting RNA expression of each of TACI, CCND1, CD10, CD30, MAL, LMO2, CD5, CD23, CD28, ICOS, and CTLA4.

4. The gene expression assay kit of claim 1, wherein the at least one marker from tumor cells of a lymphoma is selected from the group consisting of: CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, and TACI.

5. The gene expression assay kit of claim 4, wherein the assay kit further comprises probes capable of detecting RNA expression of a marker selected from the group consisting of CD23, CD28, CD3, CD5, CD8, CXCL13, GATA3, GRB, ICOS, PD1, and TBET.

6. The gene expression assay kit of claim 4, wherein the gene expression assay kit comprises a probe for detecting RNA expression of each of the following markers: CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, and TACI.

7. The gene expression assay kit of claim 6, wherein the gene expression assay kit comprises a pair of probes for detecting RNA expression of each of the following markers: CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, and TACI.

8. The gene expression assay kit of claim 7, wherein the gene expression assay kit further comprises a probe for detecting RNA expression of each of the following markers: CD23, CD28, CD3, CD5, CD8, CXCL13, GATA3, GRB, ICOS, PD1, and TBET.

9. The gene expression assay kit of claim 8, wherein the gene expression assay kit comprises a pair of probes for detecting RNA expression of each of the following markers: CD23, CD28, CD3, CD5, CD8, CXCL13, GATA3, GRB, ICOS, PD1, and TBET.

10. The gene expression assay kit of claim 6, wherein the gene expression assay kit further comprises at least one probe for detecting RNA expression of each of the following markers: ASB13, BCL6e1-BCL6e2, BCL6e3-BCL6e4, CCDC50, CD71, CD95, FGFR1, FOXP1, ITPKB, RAB7L1, SERPINA9, STAT6, TRAF1, ANXA1, APRIL, B2M, BAFF, BANK, BCMA, CARD11, CCND2, CD138, CD19, CD22, CD27, CD38, CD40, CD70, MEF2B, MS4A1, ALK, CD4, CD45RO, CXCR5, FOXP3, INFg, LAGS, PRF, TCRbeta, TCRdelta, TCRgamma, CCR4, CCR7, CD40Le2-CD40Le3, CD40Le3-CD40Le4, CD56, CD80, CD86, CTLA4, DUSP22, PRDM1, TCL1A, TRAC, XBP1, and ZAP70.

11. The gene expression assay kit of claim 10, wherein the gene expression assay kit further comprises a pair of probes for detecting RNA expression of each of the following markers: ASB13, BCL6e1-BCL6e2, BCL6e3-BCL6e4, CCDC50, CD71, CD95, FGFR1, FOXP1, ITPKB, RAB7L1, SERPINA9, STAT6, TRAF1, ANXA1, APRIL, B2M, BAFF, BANK, BCMA, CARD11, CCND2, CD138, CD19, CD22, CD27, CD38, CD40, CD70, MEF2B, MS4A1, ALK, CD4, CD45RO, CXCR5, FOXP3, INFg, LAGS, PRF, TCRbeta, TCRdelta, TCRgamma, CCR4, CCR7, CD40Le2-CD40Le3, CD40Le3-CD40Le4, CD56, CD80, CD86, CTLA4, DUSP22, PRDM1, TCL1A, TRAC, XBP1, and ZAP70.

12. The gene expression assay kit of claim 11, wherein the gene expression assay kit further comprises at least one probe for detecting RNA expression of each of the following markers: CRBN, Ialpha-Calpha, Ialpha-Cepsilon, Ialpha-Cgamma, Ialpha-Cmu, Iepsilon-Calpha, Iepsilon-Cepsilon, Iepsilon-Cgamma, Iepsilon-Cmu, Igamma-Calpha, Igamma-Cepsilon, Igamma-Cgamma, Igamma-Cmu, IGHD, IGHM, Imu-Calpha, Imu-Cepsilon, Imu-Cgamma, Imu-Cmu, JH-Calpha, JH-Cepsilon, JH-Cgamma, JH-Cmu, AIDe2-AIDe3, AIDe4-AIDe5, EBER1, HTLV1, CD163, CD68, KI67, BRAFV600E, IDH2R172K, IDH2R172T, MYD88e3-MYD88e4, MYD88L265P, RHOAG17V, XPOE571K, XPOWT, BCL6e1-Calpha, BCL6e1-Cepsilon, BCL6e1-Cgamma, BCL6e1-Cmu, Ialpha-BCL6e2, Iepsilon-BCL6e2, Igamma-BCL6e2, Imu-BCL6e2, and JH-BCL6e2.

13. The gene expression assay kit of claim 11, wherein the gene expression assay kit further comprises a pair of probes for detecting RNA expression of each of the following markers: CRBN, Ialpha-Calpha, Ialpha-Cepsilon, Ialpha-Cgamma, Ialpha-Cmu, Iepsilon-Calpha, Iepsilon-Cepsilon, Iepsilon-Cgamma, Iepsilon-Cmu, Igamma-Calpha, Igamma-Cepsilon, Igamma-Cgamma, Igamma-Cmu, IGHD, IGHM, Imu-Calpha, Imu-Cepsilon, Imu-Cgamma, Imu-Cmu, JH-Calpha, JH-Cepsilon, JH-Cgamma, JH-Cmu, AIDe2-AIDe3, AIDe4-AIDe5, EBER1, HTLV1, CD163, CD68, KI67, BRAFV600E, IDH2R172K, IDH2R172T, MYD88e3-MYD88e4, MYD88L265P, RHOAG17V, XPOE571K, XPOWT, BCL6e1-Calpha, BCL6e1-Cepsilon, BCL6e1-Cgamma, BCL6e1-Cmu, Ialpha-BCL6e2, Iepsilon-BCL6e2, Igamma-BCL6e2, Imu-BCL6e2, and JH-BCL6e2.

14. The gene expression assay kit of claim 1, wherein each probe is an RNA molecule.

15. The gene expression assay kit of claim 14, wherein each RNA molecule is 40 to 200 nucleotides long.

16. The gene expression assay kit of claim 1, wherein the assay kit comprises:
a first probe, wherein the first probe comprises a sequence that is at least 80% identical to SEQ ID NO: 29, a second probe, wherein the second probe comprises a sequence that is at least 80% identical to SEQ ID NO: 30,
a third probe, wherein the third probe comprises a sequence that is at least 80% identical to SEQ ID NO: 153, and a fourth probe, wherein the fourth probe comprises a sequence that is at least 80% identical to SEQ ID NO: 154,
a fifth probe, wherein the fifth probe comprises a sequence that is at least 80% identical to SEQ ID NO: 155, and a sixth probe, wherein the sixth probe comprises a sequence that is at least 80% identical to SEQ ID NO: 156,
a seventh probe, wherein the seventh probe comprises a sequence that is at least 80% identical to SEQ ID NO: 15, and an eighth probe, wherein the eighth probe comprises a sequence that is at least 80% identical to SEQ ID NO: 16,
a ninth probe, wherein the ninth probe comprises a sequence that is at least 80% identical to SEQ ID NO: 17, and a tenth probe, wherein the tenth probe comprises a sequence that is at least 80% identical to SEQ ID NO: 18,
an eleventh probe, wherein the eleventh probe comprises a sequence that is at least 80% the same as SEQ ID NO: 147 and a twelfth probe, wherein the twelfth probe comprises a sequence that is at least 80% identical to SEQ ID NO: 148,
a thirteenth probe, wherein the thirteenth probe comprises a sequence that is at least 80% identical to SEQ ID NO: 201 and a fourteenth probe, wherein the fourteenth probe comprises a sequence that is at least 80% identical to SEQ ID NO: 202,
a fifteenth probe, wherein the fifteenth probe comprises a sequence that is at least 80% identical to SEQ ID NO: 75 and a sixteenth probe, wherein the sixteenth probe comprises a sequence that is at least 80% identical to SEQ ID NO: 76,
a seventeenth probe, wherein the seventeenth probe comprises a sequence that is at least 80% identical to SEQ ID NO: 83 and an eighteenth probe, wherein the eighteenth probe comprises a sequence that is at least 80% identical to SEQ ID NO: 84,
a nineteenth probe, wherein the nineteenth probe comprises a sequence that is at least 80% identical to SEQ ID NO: 125 and a twentieth probe, wherein the twentieth probe comprises a sequence that is at least 80% identical to SEQ ID NO: 126,
a twenty-first probe, wherein the twenty-first probe comprises a sequence that is at least 80% identical to SEQ ID NO: 127 and a twenty-second probe, wherein the twenty-second probe comprises a sequence that is at least 80% identical to SEQ ID NO: 128,
a twenty-third probe, wherein the twenty-third probe comprises a sequence that is at least 80% identical to SEQ ID NO: 131 and a twenty-fourth probe, wherein the twenty-fourth probe comprises a sequence that is at least 80% identical to SEQ ID NO: 132,
a twenty-fifth probe, wherein the twenty-fifth probe comprises a sequence that is at least 80% identical to SEQ ID NO: 135 and a twenty-sixth probe, wherein the twenty-sixth probe comprises a sequence that is at least 80% identical to SEQ ID NO: 136,
a twenty-seventh probe, wherein the twenty-seventh probe comprises a sequence that is at least 80% identical to SEQ ID NO: 137 and a twenty-eighth probe, wherein the twenty-eighth probe comprises a sequence that is at least 80% identical to SEQ ID NO: 138,
a twenty-ninth probe, wherein the twenty-ninth probe comprises a sequence that is at least 80% identical to SEQ ID NO: 139 and a thirtieth probe, wherein the thirtieth probe comprises a sequence that is at least 80% identical to SEQ ID NO: 140,
a thirty-first probe, wherein the thirty-first probe comprises a sequence that is at least 80% identical to SEQ ID NO: 141 and a thirty-second probe, wherein the thirty-second probe comprises a sequence that is at least 80% identical to SEQ ID NO: 142,
a thirty-third probe, wherein the thirty-third probe comprises a sequence that is at least 80% identical to SEQ ID NO: 151 and a thirty-fourth probe, wherein the thirty-fourth probe comprises a sequence that is at least 80% identical to SEQ ID NO: 152,
a thirty-fifth probe, wherein the thirty-fifth probe comprises a sequence that is at least 80% identical to SEQ ID NO: 163 and a thirty-sixth probe, wherein the thirty-sixth probe comprises a sequence that is at least 80% identical to SEQ ID NO: 164,
a thirty-seventh probe, wherein the thirty-seventh probe comprises a sequence that is at least 80% identical to SEQ ID NO: 45 and a thirty-eighth probe, wherein the thirty-eighth probe comprises a sequence that is at least 80% identical to SEQ ID NO: 46,
a thirty-ninth probe, wherein the thirty-ninth probe comprises a sequence that is at least 80% identical to SEQ ID NO: 167 and a fortieth probe, wherein the fortieth probe comprises a sequence that is at least 80% identical to SEQ ID NO: 168,
a forty-first probe, wherein the forty-first probe comprises a sequence that is at least 80% identical to SEQ ID NO: 169 and a forty-second probe, wherein the forty-second probe comprises a sequence that is at least 80% identical to SEQ ID NO: 170,
a forty-third probe, wherein the forty-third probe comprises a sequence that is at least 80% identical to SEQ ID NO: 181 and a forty-fourth probe, wherein the forty-fourth probe comprises a sequence that is at least 80% identical to SEQ ID NO: 182,
a forty-fifth probe, wherein the forty-fifth probe comprises a sequence that is at least 80% identical to SEQ ID NO: 187 and a forty-sixth probe, wherein the forty-sixth probe comprises a sequence that is at least 80% identical to SEQ ID NO: 188,
a forty-seventh probe, wherein the forty-seventh probe comprises a sequence that is at least 80% identical to SEQ ID NO: 197 and a forty-eighth probe, wherein the forty-eighth probe comprises a sequence that is at least 80% identical to SEQ ID NO: 198,
a forty-ninth probe, wherein the forty-ninth probe comprises a sequence that is at least 80% identical to SEQ ID NO: 91 and a fiftieth probe, wherein the fiftieth probe comprises a sequence that is at least 80% identical to SEQ ID NO: 92,
a fifty-first probe, wherein the fifty-first probe comprises a sequence that is at least 80% identical to SEQ ID NO: 47 and a fifty-second probe, wherein the fifty-second probe comprises a sequence that is at least 80% identical to SEQ ID NO: 48,
a fifty-third probe, wherein the fifty-third probe comprises a sequence that is at least 80% identical to SEQ ID NO: 49 and a fifty-fourth probe, wherein the fifty-fourth probe comprises a sequence that is at least 80% identical to SEQ ID NO: 50,
a fifty-fifth probe, wherein the fifty-fifth probe comprises a sequence that is at least 80% identical to SEQ ID NO: 59 and a fifty-sixth probe, wherein the fifty-sixth probe comprises a sequence that is at least 80% identical to SEQ ID NO: 60,
a fifty-seventh probe, wherein the fifty-seventy probe comprises a sequence that is at least 80% identical to SEQ ID NO: 71 and a fifty-eighth probe, wherein the fifty-eighth probe comprises a sequence that is at least 80% identical to SEQ ID NO: 72,
a fifty-ninth probe, wherein the fifty-ninth probe comprises a sequence that is at least 80% identical to SEQ ID NO: 79 and a sixtieth probe, wherein the sixtieth probe comprises a sequence that is at least 80% identical to SEQ ID NO: 80,
a sixty-first probe, wherein the sixty-first probe comprises a sequence that is at least 80% identical to SEQ ID NO: 99 and a sixty-second probe, wherein the sixty-second probe comprises a sequence that is at least 80% identical to SEQ ID NO: 100,
a sixty-third probe, wherein the sixty-third probe comprises a sequence that is at least 80% identical to SEQ ID NO: 101 and a sixty-fourth probe, wherein the sixty-fourth probe comprises a sequence that is at least 80% identical to SEQ ID NO: 102,
a sixty-fifth probe, wherein the sixty-fifth probe comprises a sequence that is at least 80% identical to SEQ ID NO: 105 and a sixty-sixth probe, wherein the sixty-sixth probe comprises a sequence that is at least 80% identical to SEQ ID NO: 106,
a sixty-seventh probe, wherein the sixty-seventh probe comprises a sequence that is at least 80% identical to SEQ ID NO: 165 and a sixty-eighth probe, wherein the sixty-eighth probe comprises a sequence that is at least 80% identical to SEQ ID NO: 166, and
a sixty-ninth probe, wherein the sixty-ninth probe comprises a sequence that is at least 80% identical to SEQ ID NO: 191 and a seventieth probe, wherein the seventieth probe comprises a sequence that is at least 80% identical to SEQ ID NO: 192.

17. The gene expression assay kit of claim 16, wherein
the first probe comprises a nucleic acid sequence as set forth in SEQ ID NO: 29, the second probe comprises a nucleic acid sequence as set forth in SEQ ID NO: 30,
the third probe comprises a nucleic acid sequence as set forth in SEQ ID NO: 153, the fourth probe comprises a nucleic acid sequence as set forth in SEQ ID NO: 154,
the fifth probe comprises a nucleic acid sequence as set forth in SEQ ID NO: 155, the sixth probe comprises a nucleic acid sequence as set forth in SEQ ID NO: 156,
the seventh probe comprises a nucleic acid sequence as set forth in SEQ ID NO: 15, the eighth probe comprises a nucleic acid sequence as set forth in SEQ ID NO: 16,
the ninth probe comprises a nucleic acid sequence as set forth in SEQ ID NO: 17, the tenth probe comprises a nucleic acid sequence as set forth in SEQ ID NO: 18,
the eleventh probe comprises a nucleic acid sequence as set forth in SEQ ID NO: 147, the twelfth probe comprises a nucleic acid sequence as set forth in SEQ ID NO: 148,
the thirteenth probe comprises a nucleic acid sequence as set forth in SEQ ID NO: 201, the fourteenth probe comprises a nucleic acid sequence as set forth in SEQ ID NO: 202,
the fifteenth probe comprises a nucleic acid sequence as set forth in SEQ ID NO: 75, the sixteenth probe comprises a nucleic acid sequence as set forth in SEQ ID NO: 76,
the seventeenth probe comprises a nucleic acid sequence as set forth in SEQ ID NO: 83,
the eighteenth probe comprises a nucleic acid sequence as set forth in SEQ ID NO: 84,
the nineteenth probe comprises a nucleic acid sequence as set forth in SEQ ID NO: 125,
the twentieth probe comprises a nucleic acid sequence as set forth in SEQ ID NO: 126,
the twenty-first probe comprises a nucleic acid sequence as set forth in SEQ ID NO: 127,
the twenty-second probe comprises a nucleic acid sequence as set forth in SEQ ID NO: 128,
the twenty-third probe comprises a nucleic acid sequence as set forth in SEQ ID NO: 131,
the twenty-fourth probe comprises a nucleic acid sequence as set forth in SEQ ID NO: 132,
the twenty-fifth probe comprises a nucleic acid sequence as set forth in SEQ ID NO: 135,
the twenty-sixth probe comprises a nucleic acid sequence as set forth in SEQ ID NO: 136,
the twenty-seventh probe comprises a nucleic acid sequence as set forth in SEQ ID NO: 137, the twenty-eighth probe comprises a nucleic acid sequence as set forth in SEQ ID NO: 138,
the twenty-ninth probe comprises a nucleic acid sequence as set forth in SEQ ID NO: 139,
the thirtieth probe comprises a nucleic acid sequence as set forth in SEQ ID NO: 140,
the thirty-first probe comprises a nucleic acid sequence as set forth in SEQ ID NO: 141,
the thirty-second probe comprises a nucleic acid sequence as set forth in SEQ ID NO: 142,
the thirty-third probe comprises a nucleic acid sequence as set forth in SEQ ID NO: 151,
the thirty-fourth probe comprises a nucleic acid sequence as set forth in SEQ ID NO: 152,
the thirty-fifth probe comprises a nucleic acid sequence as set forth in SEQ ID NO: 163,
the thirty-sixth probe comprises a nucleic acid sequence as set forth in SEQ ID NO: 164,
the thirty-seventh probe comprises a nucleic acid sequence as set forth in SEQ ID NO: 45,
the thirty-eighth probe comprises a nucleic acid sequence as set forth in SEQ ID NO: 46,
the thirty-ninth probe comprises a nucleic acid sequence as set forth in SEQ ID NO: 167,
the fortieth probe comprises a nucleic acid sequence as set forth in SEQ ID NO: 168,
the forty-first probe for comprises a nucleic acid sequence as set forth in SEQ ID NO: 169,
the forty-second probe comprises a nucleic acid sequence as set forth in SEQ ID NO: 170,
the forty-third probe for comprises a nucleic acid sequence as set forth in SEQ ID NO: 181,
the forty-fourth probe comprises a nucleic acid sequence as set forth in SEQ ID NO: 182,
the forty-fifth probe for comprises a nucleic acid sequence as set forth in SEQ ID NO: 187,
the forty-sixth probe comprises a nucleic acid sequence as set forth in SEQ ID NO: 188,
the forty-seventh probe comprises a nucleic acid sequence as set forth in SEQ ID NO: 197,
the forty-eighth probe comprises a nucleic acid sequence as set forth in SEQ ID NO: 198,
the forty-ninth probe comprises a nucleic acid sequence as set forth in SEQ ID NO: 91, the fiftieth probe comprises a nucleic acid sequence as set forth in SEQ ID NO: 192,
the fifty-first probe comprises a nucleic acid sequence as set forth in SEQ ID NO: 47, the fifty-second probe comprises a nucleic acid sequence as set forth in SEQ ID NO: 48,
the fifty-third probe comprises a nucleic acid sequence as set forth in SEQ ID NO: 49, the fifty-fourth probe comprises a nucleic acid sequence as set forth in SEQ ID NO: 50,
the fifty-fifth probe comprises a nucleic acid sequence as set forth in SEQ ID NO: 59, the fifty-sixth probe comprises a nucleic acid sequence as set forth in SEQ ID NO: 60,
the fifty-seventh probe comprises a nucleic acid sequence as set forth in SEQ ID NO: 71,
the fifty-eighth probe comprises a nucleic acid sequence as set forth in SEQ ID NO: 72,
the fifty-ninth probe comprises a nucleic acid sequence as set forth in SEQ ID NO: 79, the sixtieth probe comprises a nucleic acid sequence as set forth in SEQ ID NO: 80,
the sixty-first probe comprises a nucleic acid sequence as set forth in SEQ ID NO: 99, the sixty-second probe comprises a nucleic acid sequence as set forth in SEQ ID NO: 100,
the sixty-third probe comprises a nucleic acid sequence as set forth in SEQ ID NO: 101,
the sixty-fourth probe comprises a nucleic acid sequence as set forth in SEQ ID NO: 102,
the sixty-fifth probe comprises a nucleic acid sequence as set forth in SEQ ID NO: 105,
the sixty-sixth probe comprises a nucleic acid sequence as set forth in SEQ ID NO: 106,
the sixty-seventh probe comprises a nucleic acid sequence as set forth in SEQ ID NO: 165,
the sixty-eighth probe comprises a nucleic acid sequence as set forth in SEQ ID NO: 166,
the sixty-ninth probe comprises a nucleic acid sequence as set forth in SEQ ID NO: 191,
and the seventieth probe comprises a nucleic acid sequence as set forth in SEQ ID NO: 92.

18. The gene expression assay kit of claim 1, wherein the gene expression assay kit comprises at least 224 oligonucleotide probes, and wherein each of said 224 oligonucleotide probes comprises respectively a sequence that is at least 80% identical to respectively SEQ ID NO: 1 to SEQ ID NO: 224.

19. The gene expression assay kit of claim 18, wherein each probe respectively comprises a sequence that is identical to respectively SEQ ID NO: 1 to SEQ ID NO: 224.

20. A kit comprising a gene expression assay kit of claim 1 and a ligase.

21. A method for classifying a lymphoma subtype, said method comprising:
(a) obtaining RNA from a lymphoma and from a microenvironment of said lymphoma;
(b) exposing said RNA to a gene expression assay using the gene expression assay kit of claim 1, thereby obtaining the expression levels of said RNA; and
(c) based on the expression levels of said RNA classifying said lymphoma as a subtype selected from ABC DLBCL, GCB DLBCL, PMBL, FL, MCL, SLL, and MZL.

22. A method for developing an assay distinguishing subtypes of lymphomas, said method comprising:
(a) obtaining RNA from a set of biopsy samples, wherein the set of biopsy samples comprises tissue from a plurality of lymphoma subtypes;
(b) measuring the RNA expression level of at least one marker from a plurality of lymphomas and the RNA expression level of at least one marker from a microenvironment of each of the plurality of lymphomas; and
(c) applying a machine learning algorithm to identify a signature of each lymphoma subtype.

23. The method according to claim 22, wherein an input variable of the machine learning algorithm is a biopsy sample and an output variable of this machine learning algorithm is the signature of a respective lymphoma subtype.

24. The method according to claim 23, wherein the signature of a respective lymphoma subtype is the respective lymphoma subtype from among a group of subtypes consisting of: ABC DLBCL, GCB DLBCL, PMBL, FL, MCL, SLL and MZL.

25. The method according to claim 22, wherein the machine learning algorithm is a random forest algorithm or is based on a neural network.

26. (canceled)

27. The method according to claim 22, wherein the subtypes are ABC DLBCL, GCB DLBCL, PMBL, FL, MCL, SLL and MZL.

28. The method according to claim 22, wherein said measuring comprises measuring the RNA expression level of CCND1, MYCe1-MYCe2, MYCe2-MYCe3, BCL2e1b-BCL2e2b, BCL2e1-BCL2e2, CD10, CD30, CREB3L2, CYB5R2, IL4I1, IRF4, JAK2, LIMD1, LMO2, MAL, MAML3, MYBL1, NEK6, PDL1, PDL2, PIM2, S1PR2, SH3BP5, and TACI.

29. The method according to claim 28, wherein said measuring further comprises measuring the RNA expression level of CD23, CD28, CD3, CD5, CD8, CXCL13, GATA3, GRB, ICOS, PD1, and TBET.

30. The method according to claim 29, wherein said measuring further comprises measuring the RNA expression level of ASB13, BCL6e1-BCL6e2, BCL6e3-BCL6e4, CCDC50, CD71, CD95, FGFR1, FOXP1, ITPKB, RAB7L1, SERPINA9, STAT6, TRAF1, ANXA1, APRIL, B2M, BAFF, BANK, BCMA, CARD11, CCND2, CD138, CD19, CD22, CD27, CD38, CD40, CD70, MEF2B, MS4A1, ALK, CD4, CD45RO, CXCR5, FOXP3, INFg, LAGS, PRF, TCRbeta, TCRdelta, TCRgamma, CCR4, CCR7, CD40Le2-CD40Le3, CD40Le3-CD40Le4, CD56, CD80, CD86, CTLA4, DUSP22, PRDM1, TCL1A, TRAC, XBP1, and ZAP70.

31. The method according to claim 30, wherein said measuring further comprises measuring the RNA expression level of CRBN, Ialpha-Calpha, Ialpha-Cepsilon, Ialpha-Cgamma, Ialpha-Cmu, Iepsilon-Calpha, Iepsilon-Cepsilon, Iepsilon-Cgamma, Iepsilon-Cmu, Igamma-Calpha, Igamma-Cepsilon, Igamma-Cgamma, Igamma-Cmu, IGHD, IGHM, Imu-Calpha, Imu-Cepsilon, Imu-Cgamma, Imu-Cmu, JH-Calpha, JH-Cepsilon, JH-Cgamma, JH-Cmu, AIDe2-AIDe3, AIDe4-AIDe5, EBER1, HTLV1, CD163, CD68, KI67, BRAFV600E, IDH2R172K, IDH2R172T, MYD88e3-MYD88e4, MYD88L265P, RHOAG17V, XPOE571K, XPOWT, BCL6e1-Calpha, BCL6e1-Cepsilon, BCL6e1-Cgamma, BCL6e1-Cmu, Ialpha-BCL6e2, Iepsilon-BCL6e2, Igamma-BCL6e2, Imu-BCL6e2, and JH-BCL6e2.
